SEQUENCE LISTING (89 sequences)

SEQ ID NO: 1 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 2 - protein, 198 aa, Homo sapiens
ARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDSKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 3 - protein, 198 aa, Homo sapiens
ARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 4 - protein, 10 aa, synthetic construct
VPGVGVPGVG

SEQ ID NO: 5 - protein, 4 aa, Homo sapiens
RSKR

SEQ ID NO: 6 - protein, 548 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSVPGVGVPGVGARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDSKMYQVEFKTMNAKLLMDPKRQIFLDQNMLAVIDELMQALNFNSETVPQKSSLEEPDFYKTKIKLCILLHAFRIRAVTIDRVMSYLNASGGGFWVLVVVGGVLACYSLLVTVAFIIFWVRSKR

SEQ ID NO: 7 - protein, 548 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSVPGVGVPGVGARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGFWVLVVVGGVLACYSLLVTVAFIIFWVRSKR

SEQ ID NO: 8 - protein, 570 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSVPGVGVPGVGARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDSKMYQVEFKTMNAKLLMDPKRQIFLDQNMLAVIDELMQALNFNSETVPQKSSLEEPDFYKTKIKLCILLHAFRIRAVTIDRVMSYLNASGGGFWVLVVVGGVLACYSLLVTVAFIIFWVRSKR

SEQ ID NO: 9 - protein, 570 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSVPGVGVPGVGARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGFWVLVVVGGVLACYSLLVTVAFIIFWVRSKR

SEQ ID NO: 10 - protein, 22 aa, synthetic construct
MCHQQLVISWFSLVFLASPLVA

SEQ ID NO: 11 - protein, 22 aa, synthetic construct
MLLLVTSLLLCELPHPAFLLIP

SEQ ID NO: 12 - protein, 17 aa, synthetic construct
SSGGGGSGGGGSGGGGS

SEQ ID NO: 13 - protein, 4 aa, synthetic construct
GGGS

SEQ ID NO: 14 - protein, 5 aa, synthetic construct
GGGGS

SEQ ID NO: 15 - protein, 21 aa, Homo sapiens
LCYLLDGILFIYGVILTALFL

SEQ ID NO: 16 - protein, 27 aa, Homo sapiens
FWVLVVVGGVLACYSLLVTVAFIIFWV

SEQ ID NO: 17 - protein, 28 aa, Homo sapiens
MFWVLVVVGGVLACYSLLVTVAFIIFWV

SEQ ID NO: 18 - protein, 22 aa, Homo sapiens
MALIVLGGVAGLLLFIGLGIFF

SEQ ID NO: 19 - protein, 21 aa, Homo sapiens
IYIWAPLAGTCGVLLLSLVIT

SEQ ID NO: 20 - protein, 23 aa, Homo sapiens
IYIWAPLAGTCGVLLLSLVITLY

SEQ ID NO: 21 - protein, 24 aa, Homo sapiens
IYIWAPLAGTCGVLLLSLVITLYC

SEQ ID NO: 22 - protein, 27 aa, Homo sapiens
IISFFLALTSTALLFLLFFLTLRFSVV

SEQ ID NO: 23 - protein, 21 aa, Homo sapiens
PFFFCCFIAVAMGIRFIIMVA

SEQ ID NO: 24 - protein, 10 aa, synthetic construct
GGGSSGGGSG

SEQ ID NO: 25 - protein, 12 aa, synthetic construct
ESKYGPPCPPCP

SEQ ID NO: 26 - protein, 12 aa, synthetic construct
ESKYGPPCPSCP

SEQ ID NO: 27 - protein, 22 aa, synthetic construct
ESKYGPPCPPCPGGGSSGGGSG

SEQ ID NO: 28 - protein, 39 aa, synthetic construct
IEVMYPPPYLDNEKSNGTIIHVKGKHLCPSPLFPGPSKP

SEQ ID NO: 29 - protein, 48 aa, synthetic construct
AKPTTTPAPRPPTPAPTIASQPLSLRPEACRPAAGGAVHTRGLDFACD

SEQ ID NO: 30 - protein, 45 aa, synthetic construct
TTTPAPRPPTPAPTIASQPLSLRPEACRPAAGGAVHTRGLDFACD

SEQ ID NO: 31 - protein, 129 aa, synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 229 aa, synthetic construct
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 229 aa, synthetic construct
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 41 aa, Homo sapiens
RSKRSRLLHSDYMNMTPRRPGPTRKHYQPYAPPRDFAAYRS

SEQ ID NO: 37 - protein, 41 aa, Homo sapiens
RSKRSRGGHSDYMNMTPRRPGPTRKHYQPYAPPRDFAAYRS

SEQ ID NO: 38 - protein, 42 aa, Homo sapiens
KRGRKKLLYIFKQPFMRPVQTTQEEDGCSCRFPEEEEGGCEL

SEQ ID NO: 39 - protein, 42 aa, Homo sapiens
ALYLLRRDQRLPPDAHKPPGGGSFRTPIQEEQADAHSTLAKI

SEQ ID NO: 40 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 41-43 - skipped (3 entries)

SEQ ID NO: 44 - protein, 570 aa, synthetic construct
MLLLVTSLLLCELPHPAFLLIPIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSVPGVGVPGVGARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDSKMYQVEFKTMNAKLLMDPKRQIFLDQNMLAVIDELMQALNFNSETVPQKSSLEEPDFYKTKIKLCILLHAFRIRAVTIDRVMSYLNASGGGFWVLVVVGGVLACYSLLVTVAFIIFWVRSKR

SEQ ID NO: 45 - protein, 24 aa, synthetic construct
LEGGGEGRGSLLTCGDVEENPGPR

SEQ ID NO: 46 - protein, 354 aa, synthetic construct
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 47 - protein, 323 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 48 - protein, 569 aa, synthetic construct
MLLLVTSLLLCELPHPAFLLIPIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRVEVSWEYPDTWSTPHSYFSLTFCVQVQGKSKREKKDRVFTDKTSATVICRKNASISVRAQDRYYSSSWSEWASVPCSVPGVGVPGVGARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDSKMYQVEFKTMNAKLLMDPKRQIFLDQNMLAVIDELMQALNFNSETVPQKSSLEEPDFYKTKIKLCILLHAFRIRAVTIDRVMSYLNASGGGFWVLVVVGGVLACYSLLVTVAFIIFWVRSKR

SEQ ID NO: 49 - protein, 570 aa, synthetic construct
MLLLVTSLLLCELPHPAFLLIPIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSVPGVGVPGVGARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGFWVLVVVGGVLACYSLLVTVAFIIFWVRSKR

SEQ ID NO: 50 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 51 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLYQGLSTATKDTYDALHMQALPPR

SEQ ID NO: 52 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLFQGLSTATKDTFDALHMQALPPR

SEQ ID NO: 53 - protein, 112 aa, synthetic construct
RVKFSRSADAPAYQQGQNQLYNELNLGRREEYDVLDKRRGRDPEMGGKPRRKNPQEGLYNELQKDKMAEAFSEIGMKGERRRGKGHDGLFQGLSTATKDTFDALHMQALPPR

SEQ ID NO: 54 - protein, 112 aa, synthetic construct
RVKFSRSADAPAYQQGQNQLYNELNLGRREEYDVLDKRRGRDPEMGGKPRRKNPQEGLFNELQKDKMAEAYSEIGMKGERRRGKGHDGLFQGLSTATKDTFDALHMQALPPR

SEQ ID NO: 55 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLYQGLSTATKDTFDALHMQALPPR

SEQ ID NO: 56 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTYDALHMQALPPR

SEQ ID NO: 57 - protein, 20 aa, synthetic construct
GGGGSGGGGSGGGGSGGGGS

SEQ ID NO: 58 - protein, 4 aa, synthetic construct
GGGG

SEQ ID NO: 59 - protein, 6 aa, synthetic construct
GGGSGG

SEQ ID NO: 60 - protein, 8 aa, synthetic construct
GGGSGGGS

SEQ ID NO: 61 - protein, 560 aa, synthetic construct
QVQLVQSGAEVVKPGASVKISCKASGYTFTDHAIHWVKQNPGQRLEWIGYFSPGNDDFKYSQKFQGKATLTADTSASTAYVELSSLRSEDTAVYFCTRSLNMAYWGQGTLVTVSSGSTSGGGSGGGSGGGGSSDIVMSQSPDSLAVSLGERVTLNCKSSQSVLYSSNSKNYLAWYQQKPGQSPKLLIYWASTRESGVPDRFSGSGSGTDFTLTISSVQAEDVAVYYCQQYYSYPLSFGAGTKLELKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 582 aa, synthetic construct
MLLLVTSLLLCELPHPAFLLIPQVQLVQSGAEVVKPGASVKISCKASGYTFTDHAIHWVKQNPGQRLEWIGYFSPGNDDFKYSQKFQGKATLTADTSASTAYVELSSLRSEDTAVYFCTRSLNMAYWGQGTLVTVSSGSTSGGGSGGGSGGGGSSDIVMSQSPDSLAVSLGERVTLNCKSSQSVLYSSNSKNYLAWYQQKPGQSPKLLIYWASTRESGVPDRFSGSGSGTDFTLTISSVQAEDVAVYYCQQYYSYPLSFGAGTKLELKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 115 aa, synthetic construct
QVQLVQSGAEVVKPGASVKISCKASGYTFTDHAIHWVKQNPGQRLEWIGYFSPGNDDFKYSQKFQGKATLTADTSASTAYVELSSLRSEDTAVYFCTRSLNMAYWGQGTLVTVSS

SEQ ID NO: 64 - protein, 5 aa, synthetic construct
DHAIH

SEQ ID NO: 65 - protein, 17 aa, synthetic construct
YFSPGNDDFKYSQKFQG

SEQ ID NO: 66 - protein, 6 aa, synthetic construct
SLNMAY

SEQ ID NO: 67 - protein, 113 aa, synthetic construct
DIVMSQSPDSLAVSLGERVTLNCKSSQSVLYSSNSKNYLAWYQQKPGQSPKLLIYWASTRESGVPDRFSGSGSGTDFTLTISSVQAEDVAVYYCQQYYSYPLSFGAGTKLELK

SEQ ID NO: 68 - protein, 17 aa, synthetic construct
KSSQSVLYSSNSKNYLA

SEQ ID NO: 69 - protein, 7 aa, synthetic construct
WASTRES

SEQ ID NO: 70 - protein, 9 aa, synthetic construct
QQYYSYPLS

SEQ ID NO: 71 - protein, 348 aa, synthetic construct
LEGGGEGRGSLLTCGDVEENPGPTRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 72 - protein, 652 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 544 aa, synthetic construct
DIQLTQSPSTLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGSTSGGGSGGGSGGGGSSEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 425 aa, synthetic construct
DIQLTQSPSTLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGSTSGGGSGGGSGGGGSSEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSGGGSSGGGSGMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 644 aa, synthetic construct
DIQLTQSPSTLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGSTSGGGSGGGSGGGGSSEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 31 aa, Homo sapiens
FWVLVVVGGVLACYSLLVTVAFIIFWVRSKR

SEQ ID NO: 77 - DNA, 544 bp, synthetic construct
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 78 - DNA, 79 bp, synthetic construct
  source 1..79: mol_type = other DNA
ggctagcgccaccatgctgctgctcgtgaccagcctgctgctgtgcgagctgccccaccccgcctttctgctgatcccc

SEQ ID NO: 79 - DNA, 918 bp, synthetic construct
  source 1..918: mol_type = other DNA
atatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagt

SEQ ID NO: 80 - DNA, 30 bp, synthetic construct
  source 1..30: mol_type = other DNA
gttcctggagtaggggtacctggggtgggc

SEQ ID NO: 81 - DNA, 594 bp, synthetic construct
  source 1..594: mol_type = other DNA
gccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagactcgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcc

SEQ ID NO: 82 - DNA, 81 bp, synthetic construct
  source 1..81: mol_type = other DNA
ttctgggtgctggtggtggtgggcggggtgctggcctgctacagcctgctggtgacagtggccttcatcatcttttgggtg

SEQ ID NO: 83 - DNA, 12 bp, synthetic construct
  source 1..12: mol_type = other DNA
cggagcaagcgg

SEQ ID NO: 84 - DNA, 1587 bp, synthetic construct
  source 1..1587: mol_type = other DNA
ctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagaccccagcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcacccccctaataggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccg

SEQ ID NO: 85 - DNA, 1005 bp, synthetic construct
  source 1..1005: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcc

SEQ ID NO: 86 - DNA, 45 bp, synthetic construct
  source 1..45: mol_type = other DNA
ggcggcggcgggagtggcggcgggggttctggcggaggcggtagc

SEQ ID NO: 87 - DNA, 579 bp, synthetic construct
  source 1..579: mol_type = other DNA
agggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcc

SEQ ID NO: 88 - DNA, 81 bp, synthetic construct
  source 1..81: mol_type = other DNA
ttttgggcactggtcgtggttgctggagtcctgttttgttatggcttgctagtgacagtggctctttgtgttatctggaca

SEQ ID NO: 89 - DNA, 12 bp, synthetic construct
  source 1..12: mol_type = other DNA
aatagtagaagg

(19) United States
(12) Patent Application Publication
(10) Pub. No.: US 2024/0254183 A1
(43) Pub. Date: Aug. 1, 2024

(54) MEMBRANE-BOUND IL-12 FOR CELLULAR IMMUNOTHERAPY

(71) Applicant: CITY OF HOPE, Duarte, CA (US)

(72) Inventors: Saul J. Priceman, Valley Village, CA (US); John P. MURAD, Diamond Bar, CA (US); Eric LEE, Diamond Bar, CA (US)

(21) Appl. No.: 18/612,579

(22) Filed: Mar. 21, 2024

Related U.S. Application Data
(63) Continuation of application No. PCT/US2023/064180, filed on Mar. 10, 2023.
(60) Provisional application No. 63/423,479, filed on Nov. 7, 2022.
(60) Provisional application No. 63/318,753, filed on Mar. 10, 2022.

(51) Int. Cl. C07K 14/54; C12N 5/0783

(52) U.S. Cl. CPC C07K 14/5434; C12N 5/0636; C07K 2319/02

(57) ABSTRACT

This disclosure relates to, inter alia, cells expressing both a chimeric antigen receptor (CAR) and membrane bound IL-12 and the use of these cells to target and treat cancers (e.g., solid tumors and cancers expressing CD19, CD22, BCMA, PSCA, HER2, TAG-72, and PSCA). The disclosure also relates to cells expressing a membrane bound IL-12.

### CLAIM OF PRIORITY

This application claims the benefit of International Application No. PCT/US2023/064180 filed Mar. 10, 2023, which claims benefit of U.S. Provisional Application Ser. No. 63/318,753, filed on Mar. 10, 2022 and U.S. Provisional Application Ser. No. 63/423,479, filed on Nov. 7, 2022. The entire contents of the foregoing are incorporated herein by reference.

### SEQUENCE LISTING

This application contains a Sequence Listing that has been submitted electronically as an XML file named 40056-0071001_SL_ST26.xml. The XML file, created on Mar. 20, 2024, is 92,599 bytes in size. The material in the XML file is hereby incorporated by reference in its entirety.

### TECHNICAL FIELD

This disclosure relates immune cells expressing both a membrane bound IL-12 and optionally a chimeric antigen receptor (CAR).

### BACKGROUND

Chimeric antigen receptor (CAR) engineered T cells have energized the field of cancer immunotherapy with their proven ability to treat hematological malignancies, yet the success of CAR T cells against solid tumors has been limited. The relative lack of success of CAR T cell therapy against solid tumors is likely due to a variety of factors, including: the antigen heterogeneity of solid tumors, the difficulty trafficking CAR T cells to solid tumors, and paucity of tumor selective targets. Thus, there is a need for CAR T cell therapies that are effective against solid tumors.

### SUMMARY

The present disclosure is based, at least in part, on the discovery that T cells expressing a chimeric antigen receptor (CAR T cells) and membrane-bound IL-12 exhibit improved function compared to otherwise identical CAR T cells that do not express membrane-bound IL-12 and that CAR T cells administered with cells that express membrane bound IL-12 exhibit improved function compared to CAR T cells that are not administered with cells that express membrane bound IL-12 For example, administration of cells expressing membrane-bound IL-12 can increase the ability of CAR T cells to eliminate target cells (e.g., cancer cells) that are relatively distant from the site at which the CAR T cells are administered. Accordingly, aspects of the present disclosure provide nucleic acid molecules encoding a membrane bound IL-12 having a human CD28 transmembrane domain (“mb IL-12” or “mb(28)IL-12”). In some embodiments, the mature membrane bound IL-12 includes, from amino to carboxy terminus: mature human IL-12 beta (p40) subunit, an optional first peptide linker, mature human IL-12 alpha (p35) subunit, an optional second peptide linker, human CD28 transmembrane domain. In some embodiments, all or a portion of the cytoplasmic domain of human CD28 follows the transmembrane domain. A signal sequence can precede the human IL-12 beta subunit.

In some embodiments, a useful mbIL-12 construct comprises from amino to carboxy terminus: a mature human IL-12 beta (p40) subunit, an optional first peptide linker, and a mature human IL-12 alpha (p35) subunit, an optional second peptide linker, human CD28 transmembrane domain. In some embodiments, a useful mbIL-12 construct further comprises an intracellular portion (e.g., at least 4 (e.g., 4, 5, 6, 7, 8, 9 or 10 or no more than 4, 5, 6, 7, 8, 9 or 10 contiguous amino acids of the cytoplasmic domain of human CD28 following the human CD28 transmembrane domain). In some embodiments, all or a portion of the cytoplasmic domain of human CD28 follows the transmembrane domain. A signal sequence can precede the human IL-12 beta subunit.

The p40 subunit portion can comprise or consist of the sequence:

(SEQ ID NO: 1)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFCVQVQGKSKREKKDRVFTDKTSATVICRKNASISVRAQDRYYS
SSWSEWASVPCS

A useful p40 subunit can comprise 1, 2, 3, 4, or 5 amino acid modifications, (e.g., substitutions). In some embodiments the amino acid modifications are conservative amino acid substitutions.

The p35 subunit portion can comprise or consist of the sequence:

(SEQ ID NO: 2)
ARNLPVATPDPGMFPCLHHSQNLLRAVSNMLQKARQTLEFYPCTSEEID
HEDITKDKTSTVEACLPLELTKNESCLNSRETSFITNGSCLASRKTSFM
MALCLSSIYEDSKMYQVEFKTMNAKLLMDPKRQIFLDQNMLAVIDELMQ
ALNFNSETVPQKSSLEEPDFYKTKIKLCILLHAFRIRAVTIDRVMSYLN
AS

The p35 subunit portion can comprise or consist of the sequence:

(SEQ ID NO: 3)
ARNLPVATPDPGMFPCLHHSQNLLRAVSNMLQKARQTLEFYPCTSEEID
HEDITKDKTSTVEACLPLELTKNESCLNSRETSFITNGSCLASRKTSFM
MALCLSSIYEDLKMYQVEFKTMNAKLLMDPKRQIFLDQNMLAVIDELMQ
ALNFNSETVPQKSSLEEPDFYKTKIKLCILLHAFRIRAVTIDRVMSYLN
AS

A useful p35 subunit can comprise 1, 2, 3, 4, or 5 amino acid modifications, (e.g., substitutions). For example, the amino acid S (underlined above) at position 110 of SEQ ID NOs: 2 can be modified and replaced by a polar, non-charged amino acid, and in some embodiments, the amino acid can be selected from: cysteine, threonine, tyrosine, asparagine, and glutamine. In some embodiments, the amino acid L (underlined above) at position 110 of SEQ ID NOs: 3 can be modified and replaced by a non-polar amino acid, and in some embodiments, the amino acid can be selected from: glycine, alanine, valine, isoleucine, proline, phenylalanine, methionine, and tryptophan.

The human CD28 transmembrane domain can comprise or consist of the sequence:

(SEQ ID NO: 16)
FWVLVVVGGVLACYSLLVTVAFIIFWV

The human CD28 transmembrane domain can comprise or consist of the sequence:

(SEQ ID NO: 17)
MFWVLVVVGGVLACYSLLVTVAFIIFWV

The human CD28 transmembrane domain can comprise or consist of the sequence:

(SEQ ID NO: 76)
FWVLVVVGGVLACYSLLVTVAFIIFWVRSKR

The first peptide linker can comprise or consist of, for example, the sequence

(SEQ ID NO: 4)
VPGVGVPGVG

The second peptide linker can comprise or consist of, for example, the sequence:

GGG

The first and second linker peptide linker can be have any suitable sequence. For example, the first peptide linker can consist of: 3-24 amino acids, 3-20 amino acids, 3-15 amino acids, 3-10 amino acids (e.g., 3, 4, 5, 6, 7, 8, 9, 10, 11, 12, 13, 14, 15, 16, 17, 18, 19, or 20 amino acids. Preferably, none of the amino acids are proline. For example, the second peptide linker can consist of: 3-24 amino acids, 3-20 amino acids, 3-15 amino acids, 3-10 amino acids (e.g., 3, 4, 5, 6, 7, 8, 9, 10, 11, 12, 13, 14, 15, 16, 17, 18, 19, or 20 amino acids. Preferably, none of the amino acids are proline. Suitable second peptide linkers include glycines and or a mixture of glycines and serines (e.g., GGGGSGGGGS GGGGSGGGGS (SEQ ID NO: 57), GGGG (SEQ ID NO: 58), GGGGS (SEQ ID NO: 14), GGGSGG (SEQ ID NO: 59) and GGGSGGGS (SEQ ID NO: 60). The first and second linker peptide linker can be have any suitable sequence. For example, the first peptide linker can consist of: 3-24 amino acids, 3-20 amino acids, 3-15 amino acids, 3-10 amino acids (e.g., 3, 4, 5, 6, 7, 8, 9, 10, 11, 12, 13, 14, 15, 16, 17, 18, 19, or 20 amino acids. Preferably, none of the amino acids are proline. For example, the second peptide linker can consist of: 3-24 amino acids, 3-20 amino acids, 3-15 amino acids, 3-10 amino acids (e.g., 3, 4, 5, 6, 7, 8, 9, 10, 11, 12, 13, 14, 15, 16, 17, 18, 19, or 20 amino acids. Preferably, none of the amino acids are proline. Suitable second peptide linkers include glycines and or a mixture of glycines and serines (e.g., GGGGSGGGGS GGGGSGGGGS (SEQ ID NO: 57), GGGG (SEQ ID NO: 58), GGGGS (SEQ ID NO: 14), GGGSGG (SEQ ID NO: 59) and GGGSGGGS (SEQ ID NO: 60). In some embodiments, a useful flexible linker is 1, 2, 3, 4, 5, 6, 7, 8, 9, or 10 repeats of the sequence GGGS (SEQ ID NO: 13). In some embodiments, a useful flexible linker is 1, 2, 3, 4, 5, 6, 7, 8, 9, or 10 repeats of the sequence GGGGS (SEQ ID NO: 14) or SEQ ID NO:13. In some embodiments, the linker comprises the sequence SSGGGGSGGGGSGGGGS (SEQ ID NO:12)

The human CD28 cytoplasmic domain portion can comprise or consist of the sequence:

| (SEQ ID NO: 5) | |
|---|---|
| | RSKR |

The portion of the human cytoplasmic domain when present preferably lacks signaling activity.

In some embodiments, a mature mb(28)IL-12 can comprise or consist of the sequence:

(SEQ ID NO: 6)
IWELKKDVYVVELDWYPDAPGEMVVLTCDTPEEDGITWTLDQSSE
VLGSGKTLTIQVKEFGDAGQYTCHKGGEVLSHSLLLLHKKEDGIW
STDILKDQKEPKNKTFLRCEAKNYSGRFTCWWLTTISTDLTFSVK
SSRGSSDPQGVTCGAATLSAERVRGDNKEYEYSVECQEDSACPAA
EESLPIEVMVDAVHKLKYENYTSSFFIRDIIKPDPPKNLQLKPLK
NSRQVEVSWEYPDTWSTPHSYFSLTFCVQVQGKSKREKKDRVFTD
KTSATVICRKNASISVRAQDRYYSSSWSEWASVPCSVPGVGVPGV
GARNLPVATPDPGMFPCLHHSQNLLRAVSNMLQKARQTLEFYPCT
SEEIDHEDITKDKTSTVEACLPLELTKNESCLNSRETSFITNGSC
LASRKTSFMMALCLSSIYEDSKMYQVEFKTMNAKLLMDPKRQIFL
DQNMLAVIDELMQALNFNSETVPQKSSLEEPDFYKTKIKLCILLH
AFRIRAVTIDRVMSYLNASGGGFWVLVVVGGVLACYSLLVTVAFI
IFWVRSKR

In some embodiments, a mature mb(28)IL-12 can comprise or consist of the sequence:

(SEQ ID NO: 7)
IWELKKDVYVVELDWYPDAPGEMVVLTCDTPEEDGITWTLDQSSE
VLGSGKTLTIQVKEFGDAGQYTCHKGGEVLSHSLLLLHKKEDGIW
STDILKDQKEPKNKTFLRCEAKNYSGRFTCWWLTTISTDLTFSVK
SSRGSSDPQGVTCGAATLSAERVRGDNKEYEYSVECQEDSACPAA
EESLPIEVMVDAVHKLKYENYTSSFFIRDIIKPDPPKNLQLKPLK
NSRQVEVSWEYPDTWSTPHSYFSLTFCVQVQGKSKREKKDRVFTD
KTSATVICRKNASISVRAQDRYYSSSWSEWASVPCSVPGVGVPGV
GARNLPVATPDPGMFPCLHHSQNLLRAVSNMLQKARQTLEFYPCT
SEEIDHEDITKDKTSTVEACLPLELTKNESCLNSRETSFITNGSC
LASRKTSFMMALCLSSIYEDLKMYQVEFKTMNAKLLMDPKRQIFL
DQNMLAVIDELMQALNFNSETVPQKSSLEEPDFYKTKIKLCILLH
AFRIRAVTIDRVMSYLNASGGGFWVLVVVGGVLACYSLLVTVAFI
IFWVRSKR

In the above mature mb(28)IL-12 sequences, the first and second peptide linker sequences are underlined; the human CD28 transmembrane domain sequence is in bold and the portion of human CD28 cytoplasmic domain sequence is in italics. A useful mb(28)IL-12 can comprise SEQ ID NO:6 with 1, 2, 3, 4, 5 amino acid modification or comprise a sequence with 80, 85, 88, 89, 90, 91, 92, 93, 94, 95, 96, 97, 98, 99, or 100% identity to SEQ ID NO:6. A useful mb(28)IL-12 can comprise SEQ ID NO:7 with 1, 2, 3, 4, 5 amino acid modification or comprise a sequence with 80, 85, 88, 89, 90, 91, 92, 93, 94, 95, 96, 97, 98, 99, or 100% identity to SEQ ID NO:7.

The mature sequence can be preceded by a signal sequence suitable for directing secretion to the surface of a human cell. For example, the signal sequence can comprise or consist of the sequence: MCHQQLVISWFSLVFLASPLVA (SEQ ID NO: 10).

In one aspect, a mbIL-12 with a signal sequence can comprise or consist of the sequence:

(SEQ ID NO: 8)
MCHQQLVISWFSLVFLASPLVAIWELKKDVYVVELDWYPDAPGEM
VVLTCDTPEEDGITWTLDQSSEVLGSGKTLTIQVKEFGDAGQYTC
HKGGEVLSHSLLLLHKKEDGIWSTDILKDQKEPKNKTFLRCEAKN
YSGRFTCWWLTTISTDLTFSVKSSRGSSDPQGVTCGAATLSAERV
RGDNKEYEYSVECQEDSACPAAEESLPIEVMVDAVHKLKYENYTS
SFFIRDIIKPDPPKNLQLKPLKNSRQVEVSWEYPDTWSTPHSYFS
LTFCVQVQGKSKREKKDRVFTDKTSATVICRKNASISVRAQDRYY
SSSWSEWASVPCSVPGVGVPGVGARNLPVATPDPGMFPCLHHSQN
LLRAVSNMLQKARQTLEFYPCTSEEIDHEDITKDKTSTVEACLPL
ELTKNESCLNSRETSFITNGSCLASRKTSFMMALCLSSIYEDSKM
YQVEFKTMNAKLLMDPKRQIFLDQNMLAVIDELMQALNFNSETVP
QKSSLEEPDFYKTKIKLCILLHAFRIRAVTIDRVMSYLNASGGGF
WVLVVVGGVLACYSLLVTVAFIIFWVRSKR

In this sequence the signal sequence is underlined.

In one aspect, mb(DC28)IL-12 with a signal sequence can comprise or consist of the sequence:

(SEQ ID NO: 9)
MCHQQLVISWFSLVFLASPLVAIWELKKDVYVVELDWYPDAPGEM
VVLTCDTPEEDGITWTLDQSSEVLGSGKTLTIQVKEFGDAGQYTC
HKGGEVLSHSLLLLHKKEDGIWSTDILKDQKEPKNKTFLRCEAKN
YSGRFTCWWLTTISTDLTFSVKSSRGSSDPQGVTCGAATLSAERV
RGDNKEYEYSVECQEDSACPAAEESLPIEVMVDAVHKLKYENYTS
SFFIRDIIKPDPPKNLQLKPLKNSRQVEVSWEYPDTWSTPHSYFS
LTFCVQVQGKSKREKKDRVFTDKTSATVICRKNASISVRAQDRYY
SSSWSEWASVPCSVPGVGVPGVGARNLPVATPDPGMFPCLHHSQN
LLRAVSNMLQKARQTLEFYPCTSEEIDHEDITKDKTSTVEACLPL
ELTKNESCLNSRETSFITNGSCLASRKTSFMMALCLSSIYEDLKM
YQVEFKTMNAKLLMDPKRQIFLDQNMLAVIDELMQALNFNSETVP
QKSSLEEPDFYKTKIKLCILLHAFRIRAVTIDRVMSYLNASGGGF
WVLVVVGGVLACYSLLVTVAFIIFWVRSKR

In this sequence, the signal sequence is underlined.

In other aspects, the signal sequence can comprise or consist of:

(SEQ ID NO: 11)
MLLLVTSLLLCELPHPAFLLIP

In some embodiments, a mbIL-12 with a signal sequence can comprise or consist of the sequence:

(SEQ ID NO: 48)
MLLLVTSLLLCELPHPAFLLIPIWELKKDVYVVELDWYPDAPGEM
VVLTCDTPEEDGITWTLDQSSEVLGSGKTLTIQVKEFGDAGQYTC
HKGGEVLSHSLLLLHKKEDGIWSTDILKDQKEPKNKTFLRCEAKN
YSGRFTCWWLTTISTDLTFSVKSSRGSSDPQGVTCGAATLSAERV
RGDNKEYEYSVECQEDSACPAAEESLPIEVMVDAVHKLKYENYTS
SFFIRDIIKPDPPKNLQLKPLKNSRVEVSWEYPDTWSTPHSYFSL
TFCVQVQGKSKREKKDRVFTDKTSATVICRKNASISVRAQDRYYS
SSWSEWASVPCSVPGVGVPGVGARNLPVATPDPGMFPCLHHSQNL
LRAVSNMLQKARQTLEFYPCTSEEIDHEDITKDKTSTVEACLPLE
LTKNESCLNSRETSFITNGSCLASRKTSFMMALCLSSIYEDSKMY
QVEFKTMNAKLLMDPKRQIFLDQNMLAVIDELMQALNFNSETVPQ
KSSLEEPDFYKTKIKLCILLHAFRIRAVTIDRVMSYLNASGGGFW
VLVVVGGVLACYSLLVTVAFIIFW
VRSKR

In this sequence, the signal sequence is underlined.

In some embodiments, a mbIL-12 with a signal sequence can comprise or consist of the sequence:

(SEQ ID NO: 49)
MLLLVTSLLLCELPHPAFLLIPIWELKKDVYVVELDWYPDAPGEM
VVLTCDTPEEDGITWTLDQSSEVLGSGKTLTIQVKEFGDAGQYTC
HKGGEVLSHSLLLLHKKEDGIWSTDILKDQKEPKNKTFLRCEAKN
YSGRFTCWWLTTISTDLTFSVKSSRGSSDPQGVTCGAATLSAERV
RGDNKEYEYSVECQEDSACPAAEESLPIEVMVDAVHKLKYENYTS
SFFIRDIIKPDPPKNLQLKPLKNSRQVEVSWEYPDTWSTPHSYFS
LTFCVQVQGKSKREKKDRVFTDKTSATVICRKNASISVRAQDRYY
SSSWSEWASVPCSVPGVGVPGVGARNLPVATPDPGMFPCLHHSQN
LLRAVSNMLQKARQTLEFYPCTSEEIDHEDITKDKTSTVEACLPL
ELTKNESCLNSRETSFITNGSCLASRKTSFMMALCLSSIYEDLKM
YQVEFKTMNAKLLMDPKRQIFLDQNMLAVIDELMQALNFNSETVP
QKSSLEEPDFYKTKIKLCILLHAFRIRAVTIDRVMSYLNASGGGF
WVLVVVGGVLACYSLLVTVAFIIFWVRSKR

In this sequence, the signal sequence is underlined.

In some embodiments, a mb(28)IL12 described herein can comprise a sequence selected from SEQ ID NO:6, 7, 8, 9, 48, 49, or a variant of each having 1, 2, 3, 4, 5 amino acid modifications. In some embodiments, a mb(28)IL12 described herein can comprise a sequence 80, 85, 88, 89, 90, 91, 92, 93, 94, 95, 96, 97, 98, 99, or 100% identity to any one of SEQ ID NO: 6, 7, 8, 9, 48, and 49. In some embodiments, the sequence FWVLVVVGGVLACYSLLVTVAFIIFWV (SEQ ID NO: 16) can be replaced with MFWVLVVVGGVLACYSLLVTVAFIIFWV (SEQ ID NO: 17) or FWVLVVVGGVLACYSLLVTVAFIIFWVRSKR(SEQ ID NO: 76). In some embodiments, the portion of the human cytoplasmic domain when present preferably lacks signaling activity.

Nucleic acids described herein can comprise a sequence encoding a sequence selected from SEQ ID NO:6, 7, 8, 9, 48, 49, or a variant of each having 1, 2, 3, 4, 5 amino acid modifications. In some embodiments, a nucleic acid can comprise a sequence encoding a sequence having 80, 85, 88, 89, 90, 91, 92, 93, 94, 95, 96, 97, 98, 99, or 100% identity to any one of SEQ ID NO:6, 7, 8, 9, 48, and 49. In some embodiments, the sequence FWVLVVVGGVLACYSLLVTVAFIIFWV (SEQ ID NO: 16) can be replaced with MFWVLVVVGGVLACYSLLVTVAFIIFWV (SEQ ID NO: 17) or FWVLVVVGGVLACYSLLVTVAFIIFWVRSKR(SEQ ID NO: 76).

Disclosed herein, inter alia, are nucleic acid molecules encoding membrane bound IL-12 (mbIL-12), comprising, from amino to carboxy terminus: mature human IL-12 p40 subunit, an optional first peptide linker, mature human IL-12 p35 subunit, an optional second peptide linker, human CD28 transmembrane domain. In some embodiments, the mbIL-12 further comprises at least 4 contiguous amino acids of the cytoplasmic domain of human CD28 following the human CD28 transmembrane domain. In some embodiments, the amino acid sequence of the mature human IL-12 p40 subunit comprises SEQ ID NO: 1. In some embodiments, the amino acid sequence of the mature human IL-12 p35 comprises SEQ ID NO: 2 or 3. In some embodiments, the first peptide linker comprises the amino acid sequence of SEQ ID NO: 4. In some embodiments, the second peptide linker comprises the amino acid sequence GGG. In some embodiments, the human CD28 transmembrane domain comprises the amino acid sequence of SEQ ID NO: 16 or 17 or 76. In some embodiments, the at least 4 contiguous amino acids of the cytoplasmic domain of human CD28 following the human CD28 transmembrane domain comprises the amino acid sequence of SEQ ID NO: 5. In some embodiments, the mbIL-12 comprises the amino acid sequence of SEQ ID NO: 6 or 7.

Also disclosed herein are populations of human immune cells (e.g., T cells, NK cells, dendritic cells, macrophages or monocytes) comprising a nucleic acid encoding a mb(28)IL12 described herein. In some embodiments, a population of human immune cells comprising a nucleic acid molecule encoding a mb IL-12, comprising, from amino to carboxy terminus: a mature human IL-12 p40 subunit, a first peptide linker, a mature human IL-12 p35 subunit, a second peptide linker, and a human CD28 transmembrane domain. In some embodiments, the mbIL-12 further comprises at least a portion of the cytoplasmic domain of human CD28 following the human CD28 transmembrane domain. In some embodiments, the amino acid sequence of the mature human IL-12 p40 subunit comprises SEQ ID NO: 1. In some embodiments, the amino acid sequence of the mature human IL-12 p35 comprises SEQ ID NO: 2 or 3. In some embodiments, the first peptide linker comprises the amino acid sequence of SEQ ID NO: 4. In some embodiments, the second peptide linker comprises the amino acid sequence GGG. In some embodiments, the human CD28 transmembrane domain comprises the amino acid sequence of SEQ ID NO: 16 or 17 or 76. In some embodiments, the cytoplasmic domain of a human CD28 transmembrane domain comprises at least 4 contiguous amino acids of a cytoplasmic domain of human CD28 following the human CD28 transmembrane domain or comprises the amino acid sequence of SEQ ID NO: 5. In some embodiments, the mbIL-12 comprises the amino acid sequence of SEQ ID NO: 6 or 7. In some embodiments, the population of immune cells is a population of T cells, a population of NK cells, a population of dendritic cells, a population of macrophages, or a population of monocytes.

In some embodiments, a population of immune cells comprise a nucleic acid comprising a sequence encoding a mb(28)IL12 and comprise a nucleic acid comprising a sequence encoding a chimeric antigen receptor (CAR). In some embodiments, the chimeric antigen receptor is targeted to a human antigen selected from the group consisting of: CD19, CD22, BCMA, PSCA, HER2, and TAG-72. In some embodiments, the chimeric antigen receptor is targeted to human TAG-72. In some embodiments, the chimeric antigen receptor comprises: an scFv targeting Tag-72, a spacer, a transmembrane domain, a CD28 co-stimulatory domain or a 41-BB co-stimulatory domain, and a human CD3 (signaling domain. In some embodiments, the transmembrane domain is selected from: a CD4 transmembrane domain, a CD8 transmembrane domain, a CD28 transmembrane domain. In some embodiments, the TAG72 scFV is selected from IDEC, V15, V59 and V59_V15. In some embodiments, the transmembrane domain is a human CD28 transmembrane domain. In some embodiments, the spacer region comprises an amino acid sequence selected from the group consisting of SEQ ID NOs: 2-12. In some embodiments, the spacer comprises an IgG hinge region. In some embodiments, the spacer consists of 10-50 amino acids. In some embodiments, the 4-1 BB costimulatory domain comprises the amino acid sequence of SEQ ID NO: 38. In some embodiments, the CD3ζ signaling domain comprises the amino acid sequence of SEQ ID NO:35. In some embodiments, a linker of 3 to 15 amino acids is located between the 4-1 BB costimulatory domain and the CD3 (signaling domain or variant thereof.

In some cases, the mbIL-12 constructs described herein is expressed with a CAR, e.g., a CAR targeted to a solid tumor antigen, e.g., HER2 or PSCA.

Described herein, inter alia, are populations of human immune cells (e.g., T cells, NK cells, dendritic cells, macrophages or monocytes) expressing a membrane bound IL-12 (mbIL-12) comprising, from amino to carboxy terminus: a mature human IL-12 p40 subunit, a first peptide linker, a mature human IL-12 p35 subunit, a second peptide linker, and a human CD28 transmembrane domain. In some embodiments, the mbIL-12 further comprises at least a portion of the cytoplasmic domain of human CD28 following the human CD28 transmembrane domain. In some embodiments, the amino acid sequence of the mature human IL-12 p40 subunit comprises SEQ ID NO: 1. In some embodiments, the amino acid sequence of the mature human IL-12 p35 comprises SEQ ID NO: 2 or 3. In some embodiments, the first peptide linker comprises the amino acid sequence of SEQ ID NO: 4. In some embodiments, the second peptide linker comprises the amino acid sequence GGG. In some embodiments, the human CD28 transmembrane domain comprises the amino acid sequence of SEQ ID NO: 16 or 17 or 76. In some embodiments, the cytoplasmic domain of a human CD28 transmembrane domain comprises at least 4 contiguous amino acids of a cytoplasmic domain of human CD28 following the human CD28 transmembrane domain or comprises the amino acid sequence of SEQ ID NO: 5. In some embodiments, the mbIL-12 comprises the amino acid sequence of SEQ ID NO: 6 or 7. In some embodiments, the population of immune cells is a population of T cells, a population of NK cells, a population of dendritic cells, a population of macrophages, or a population of monocytes.

Also described herein are methods for treating cancer comprising administering the human immune cells comprising a nucleic acid encoding a mb(28)IL12 described herein and human immune cells expressing a chimeric antigen receptor. In some embodiments, the immune cells comprising a nucleic acid encoding a mb(28)IL12 described herein are administered before the immune cells expressing a chimeric antigen receptor are administered. In some embodiments, the immune cells comprising a nucleic acid encoding a mb(28)IL12 described herein are administered together with the immune cells expressing a chimeric antigen receptor are administered. In some embodiments, the cancer is a solid tumor. In some embodiments the cancer expresses CD19, CD22, BCMA, PSCA, HER2, TAG-72, or PSCA.

Also described herein are methods for treating cancer comprising administering the human immune cells expressing a mb(28)IL12 described herein and human immune cells expressing a chimeric antigen receptor. In some embodiments, the immune cells expressing a mb(28)IL12 described herein are administered before the immune cells expressing a chimeric antigen receptor are administered. In some embodiments, the immune cells expressing a mb(28)IL12 described herein are administered together with the immune cells expressing a chimeric antigen receptor are administered. In some embodiments, the cancer is a solid tumor. In some embodiments the cancer expresses CD19, CD22, BCMA, PSCA, HER2, TAG-72, or PSCA.

Also described herein, inter alia, are populations of human immune cells (e.g., T cells, NK cells, dendritic cells, macrophages or monocytes) expressing a membrane bound IL-12 (mbIL-12) and a CAR targeted to a cancer antigen, wherein the mbIL-12 comprises, from amino to carboxy terminus: mature human IL-12 p40 subunit, a first peptide linker, mature human IL-12 p35 subunit, a second peptide linker, human CD28 transmembrane domain, and wherein the CAR comprises an scFv targeting a cancer antigen, a spacer, a transmembrane domain, a CD28 co-stimulatory domain or a 4-1 BB co-stimulatory domain, and a human CD3 ζ signaling domain. In some embodiments, the mbIL-12 further comprises a cytoplasmic domain of human CD28 following the human CD28 transmembrane domain. In some embodiments, the amino acid sequence of the mature human IL-12 p40 subunit comprises SEQ ID NO: 1. In some embodiments, the amino acid sequence of the mature human IL-12 p35 comprises SEQ ID NO: 2 or 3. In some embodiments, the first peptide linker comprises the amino acid sequence of SEQ ID NO: 4. In some embodiments, the second peptide linker comprises the amino acid sequence GGG. In some embodiments, the human CD28 transmembrane domain comprises the amino acid sequence of SEQ ID NO: 16 or 17 or 76. In some embodiments, the cytoplasmic domain of a human CD28 transmembrane domain comprises at least 4 contiguous amino acids of a cytoplasmic domain of human CD28 following the human CD28 transmembrane domain or comprises the amino acid sequence of SEQ ID NO: 5. In some embodiments, the mbIL-12 comprises the amino acid sequence of SEQ ID NO: 6 or 7. In some embodiments, the population of human immune cells is a population of human T cells or human NK cells. In some embodiments, the cancer antigen from a solid tumor or a metastasis of a solid tumor. In some embodiments, the cancer antigen is selected from the group consisting of: CD19, CD22, BCMA, PSCA, HER2, and TAG-72. In some embodiments, wherein the transmembrane domain is selected from: a CD4 transmembrane domain, a CD8 transmembrane domain, a CD28 transmembrane domain. In some embodiments, the TAG72 scFV is selected from IDEC, V15, V59 and V59 V15. In some embodiments, the transmembrane domain is a human CD28 transmembrane domain. In some embodiments, the spacer region comprises an amino acid sequence selected from the group consisting of SEQ ID NOs: 2-12. In some embodiments, the spacer comprises an IgG hinge region. In some embodiments, the spacer consists of 10-50 amino acids. In some embodiments, the 4-1 BB costimulatory domain comprises the amino acid sequence of SEQ ID NO: 38. In some embodiments, the CD3ζ signaling domain comprises the amino acid sequence of SEQ ID NO:35. In some embodiments, a linker of 3 to 15 amino acids is located between the 4-1 BB costimulatory domain and the CD3 ζ (signaling domain or variant thereof.

In some embodiments, a CAR can comprise any one of SEQ ID NOs: 61, 62, 72, 73, 74, 75, or a variant of each having 1, 2, 3, 4, 5, 6, 7, 8, 9, or 10 amino acid modifications. In some embodiments, a CAR can comprise a sequence having 80, 85, 88, 89, 90, 91, 92, 93, 94, 95, 96, 97, 98, 99, or 100% identity to any one of SEQ ID NOs: 61, 62, 72, 73, 74, and 75.

Also described herein are methods of making a population of T cells (including, e.g., gamma/delta T cells), monocytes, macrophages, dendritic cells or NK cells expressing a mb(28)IL-12 construct and optionally a CAR.

Also described herein are vectors comprising any of the nucleic acid constructs described herein. In some embodiments, the vector is a viral vector. In some embodiments, the vector is a lentiviral vector. In some embodiments, the vector comprises a sequence that encodes any of the mb(28)IL-12 constructs disclosed herein. In some embodiments, a vector comprises a sequence that encodes a sequence selected from SEQ ID NO:6, 7, 8, 9, 48, 49, or a variant of each having 1, 2, 3, 4, 5 amino acid modifications. In some embodiments, a vector comprises a sequence that encodes a sequence having 80, 85, 88, 89, 90, 91, 92, 93, 94, 95, 96, 97, 98, 99, or 100% identity to any one of SEQ ID NO: 6, 7, 8, 9, 48, and 49.

Also disclosed herein are method for treating cancer comprising administering to a patient in need thereof any population of human immune cells (e.g., macrophages, monocytes, T cells or NK cells) described herein. In some embodiments, the patient is suffering from a solid tumor. In some embodiments, the solid tumor is metastatic. In some embodiments, the population of human immune cells (e.g., macrophages, monocytes, T cells or NK cells) is administered intravenously. In some embodiments, the population of human immune cells (e.g., macrophages, monocytes, T cells or NK cells) (e.g., macrophages, monocytes, T cells or NK cells) is administered intraperitoneally. In some embodiments, the population of human immune cells (e.g., macrophages, monocytes, T cells or NK cells) is administered intraperitoneally in the region of the solid tumor. In some embodiments, the solid tumor is ovarian cancer and the chimeric antigen receptor is targeted to TAG72. In some embodiments, the population of human immune cells (e.g., macrophages, monocytes, T cells or NK cells) is allogenic. In some embodiments, the population of human immune cells (e.g., macrophages, monocytes, T cells or NK cells) is autologous. In some embodiments, a therapeutically effective amount of a population of immune cells (e.g., macrophages, monocytes, T cells or NK cells) is administered to the patient. In some embodiments, the cancer is a solid tumor. In some embodiments, the cancer expresses CD19, CD22, BCMA, PSCA, HER2, TAG-72, or PSCA

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1. TAG72 expression on ovarian tumor cell lines. Flow cytometric analysis of TAG72 expression on human OVCAR8 (TAG72-negative), human OVCAR3 (TAG72-positive), human OV90 (TAG72-positive), human OVCAR8-sTn (TAG72-positive) tumor cell lines.

FIG. 2. In vitro and in vivo analysis of TAG72-CAR T cells with varying scFv. (a) Diagram of the lentiviral construct with a TAG72-CAR containing three different humanized scFvs (based on CC49 clone) targeting TAG72, with dCH2 extracellular spacer domain (dCH2), CD4 transmembrane (4tm), and intracellular 4-1 BB costimulatory domain (BBz) followed by a cytolytic domain (CD3z). A truncated non-signaling CD19 (CD19t), separated from the CAR sequence by a ribosomal skip sequence (T2A), was expressed for identifying lentivirally transduced T cells. (b-c) Untransduced (UTD) and three TAG72-CAR T cells positively enriched for CD19t were evaluated by flow cytometry for CD19t expression to detect lentiviral transduction of CARs (b), or Protein L to detect the scFv (c). (d-g) Quantification of tumor cell killing (d), IFNγ production (e), T cell proliferation (f), and tumor cell killing (g) by the three TAG72-CAR T cells relative to UTD T cells at an E:T ratio of 1:2, following a 24 or 72 hour co-culture with antigen-positive and −negative tumor targets as described in Materials and Methods. (h) Quantification of flux from i.p. OV90(eGFP/ffluc) tumor-bearing mice treated i.p. with UTD or TAG72-CAR T cells. n=6-7 per group.

FIG. 3 In vitro optimization of TAG72-CAR T cells. (a) Diagram of the lentiviral construct with TAG72-CAR containing the humanized scFv targeting TAG72, varying extracellular spacer domains (EQ, dCH2, CD8h, HL, L), transmembrane domains (CD4tm, CD8tm, CD28tm), and intracellular costimulatory domains (4-1 BB, CD28) followed by a cytolytic domain (CD3z). A truncated non-signaling CD19 (CD19t), separated from the CAR sequence by a ribosomal skip sequence (T2A), was expressed for identifying lentivirally transduced T cells. (b-d) Untransduced (UTD) and 7 different TAG72-CAR T cells positively enriched for CD19t were evaluated by flow cytometry for CD19t expression to detect lentiviral transduction of CARs (b), fragment constant (Fc) derived spacer containing CARs (c), or Protein L to detect the scFv (d). (e-j) In vitro tumor cell killing activity relative to UTD (e), expression of CD137 activation and PD-1 exhaustion (f-g), and T cell proliferation in fold change (h) by flow cytometry, and IFNγ and IL-2 cytokine production by ELISA (i-j), of CAR T cells against tumor targets (TAG72-OVCAR8; TAG72+ OVCAR3, OV90, and OVCAR8-sTn) after 72 hours of coculture at an effector:target (E:T) ratio of 1:4. (k) TAG72-CAR T cell killing of OV90 cells measured by xCELLigence over 10 days (E:T=1:20). (I) Schema of repetitive tumor cell challenge assay (top). TAG72-CAR T cells were cocultured with OV90 cells (E:T=1:2) and rechallenged with OV90 cells every two days. Remaining viable tumor cells and fold change in TAG72-CAR T cells were quantified as described in Materials and Methods prior to each tumor cell rechallenge.

FIG. 4 Locoregional intraperitoneal delivery of TAG72-dCH2(28tm)BBz CAR T cells reduces tumor burden and extends overall survival in vivo. (a) Bioluminescent flux imaging of intraperitoneal (i.p.) OVCAR3(eGFP/ffluc) tumor-bearing female NSG mice treated i.p. with UTD or indicated TAG72-CAR T cells. (b) Quantification of flux (individual mice in each group) from treated OVCAR3 tumor-bearing mice. UTD (n=8/group); TAG72-CAR T cells (n=10/group). (c) Flow cytometric analysis of the frequency of human CD45+ (hCD45) and mouse CD45+(mCD45) cells in the peritoneal cavity of tumor-bearing mice at day 14 post-treatment (top); human epithelial cell adhesion molecule+(EpCAM) and tumor associated glycoprotein-72+ (TAG72) tumor cells in the i.p. cavity of tumor-bearing mice at day 62 post-treatment (bottom). (d) Quantification of TAG72-CAR T cells per uL of peripheral blood at 7, 14, and 28 days post-treatment. N=5/group.

FIG. 5 Regional intraperitoneal delivery of TAG72-dCH2(28tm)BBz CAR T cells reduces tumor burden and extends overall survival in vivo. (a) Bioluminescent flux imaging of mice treated i.p. with UTD or TAG72-CAR T cells. (b-c) Quantification of flux (b, individual mice per group; c, averages) from i.p. OV90(eGFP/ffluc) tumor-bearing mice treated i.p. with UTD or TAG72-CAR T cells. n=9 mice per group. (d) Kaplan-Meier survival for UTD, scFv-less, and TAG72-CAR T cell treated mice. n=8-9 mice/group. (e) Quantification of TAG72-CAR T cells per uL of peripheral blood at 7, 14, and 28 days post-treatment. n=5/group.

FIG. 6 In vitro safety of TAG72-CAR T cells against normal human cell lines. (a) Flow cytometric analysis of TAG72 expression on the cell surface of TAG72-negative (OVCAR8) tumor cells, TAG72-positive (OV90) tumor cells, and indicated primary human normal cells. (b-c) Quantification of CD137 activation (b), and tumor and normal cell killing by TAG72-dCH2(28tm)BBz CAR T cells relative to UTD T cells at varying E:T ratios (c), assessed by flow cytometry following a 48 hour coculture with indicated cells as described in Materials and Methods. Data are of five independent donors.

FIG. 7 Membrane-bound IL-12 engineered TAG72-CAR T cells induce higher IFNγ, T cell expansion, and anti-tumor activity in vitro. (a-c) Tumor cell killing of OV90 cells by TAG72-CAR T cells (E:T=1:20) with addition of varying concentrations of anti-IFNγR1 blocking antibody, isotype control, and recombinant human IL-12 cytokine measured by xCELLigence over 10 days (a). At day 10, IFNγ levels in supernatants were quantified by ELISA (b) and remaining T cell counts were analyzed by flow cytometry (c). (d-h) Diagram of the lentiviral construct with two versions of membrane-bound IL-12 (mbIL12) containing B7.1 or CD28 transmembrane domains (d). Flow cytometric analysis of IL-12 surface expression on indicated T cells (e). TAG72-CAR/mbIL12 T cell killing of OV90 cells (E:T=1:20) measured by xCELLigence over 10 days (f). At day 10, remaining T cell counts were analyzed by flow cytometry (g) and IFNγ levels in supernatants were quantified by ELISA (h). (i) Schema of repetitive tumor cell challenge assay (top). TAG72-CAR/mbIL12(CD28tm) T cells were cocultured with OV90 cells (E:T=1:3) and rechallenged with OV90 cells every two days. Remaining viable tumor cells and fold change in TAG72-CAR T cells were quantified as described in Materials and Methods prior to each tumor cell rechallenge.

FIG. 8 TAG72-CAR T cell anti-tumor activity is regulated by IFNγ signaling. Tumor cell killing of OVCAR3 cells by TAG72-CAR T cells (E:T=1:50) with varying concentrations of anti-IFNγR1 blocking antibody, isotype control, and recombinant human IL-12 cytokine measured by xCELLigence.

FIG. 9 Supplemental FIG. 6. Inhibition of IFNγ signaling dampens repetitive tumor cell killing by TAG72-CAR T cells. Schema of repetitive tumor cell challenge assay (top). TAG72-CAR T cells were cocultured with OV90 cells (E:T=1:2) and rechallenged with OV90 cells every three days. Remaining viable tumor cells and TAG72-CAR T cells were quantified as described in Materials and Methods prior to each tumor cell rechallenge.

FIG. 10 Antigen-dependent IL-12 signaling in TAG72-CAR T cells. (a) Intracellular flow cytometric analysis of phosphorylated STAT4 (pSTAT4, pY693) in response to TAG72 and/or recombinant huIL12 (10 ng/mL) at indicated timepoints. (b) Quantification of pSTAT4 in (a). (c) Flow cytometric analysis of surface expression of mbIL12 on TAG72-CAR T cells stimulated with plate-bound TAG72 (100 U/mL) or control antigen (PSCA, 2.5 ug/mL). (d) Flow cytometric analysis of surface or intracellular expression of mbIL12 in TAG72-CAR T cells stimulates with varying concentrations of plate-bound TAG72. (e) Flow cytometric analysis of surface expression of mbIL12 on total, or gated on CD137+ or CD137-populations, of TAG72-CAR T cells stimulated with plate-bound TAG72 (100 U/mL). Table inset: CD137+ expression on TAG72-CAR T cells following stimulation with varying concentrations of plate-bound TAG72. (f) Intracellular flow cytometric analysis of phosphorylated STAT3 (pSTAT3, pY705) (left) and pSTAT4 (right) in TAG72-CAR and TAG72-CAR/mbIL12 T cells stimulated with varying concentrations of plate-bound TAG72 or recombinant huIL12 (10 ng/mL). (g) Intracellular flow cytometric analysis of pSTAT4 in TAG72-CAR T cells cocultured with HT1080 (TAG72-) cells transduced with mbIL12. Cells were stimulated with Immunocult CD3/CD28 per manufacturer recommendation. Cells were gated on CAR T cells and evaluated for pSTAT4. (h) mRNA levels of IL-12 in TAG72-CAR and TAG72-CAR/mbIL12 T cells stimulated with TAG72-OVCAR8 or TAG72+OV90 tumors overnight prior to RNA isolation and bulk RNAseq.

FIG. 11 Locoregional intraperitoneal delivery of TAG72-CAR/mbIL12 T cells reduces tumor burden and increases regional and systemic CAR T cell persistence in vivo. (a) Bioluminescent flux imaging of i.p. OVCAR3(eGFP/ffluc) tumor-bearing mice treated i.p. with CD19-CAR, CD19-CAR/mbIL12, TAG72-CAR or TAG72-CAR/mbIL12 T cells. (b) Quantification of flux (individual mice per group) from mice treated i.p. with CD19-CAR T cells (n=6/group), CD19-CAR/mbIL12 T cells (n=6/group), TAG72-CAR T cells (n=7/group) and TAG72-CAR/mbIL12 T cells (n=7/group). (c) Flow cytometric analysis of human CD45+ (hCD45) and mouse CD45+ (mCD45) cells in the peritoneal cavity of tumor-bearing mice at week 2 (top) or week 4 (bottom) post-treatment. (d) Quantification of TAG72-CAR T cells per uL of peritoneal ascites (left) at weeks 2 and 4 post-treatment. n=2 per group. Quantification of TAG72-CAR T cells per uL of peripheral blood (right) at weeks 1, 2, and 4 post-treatment. n=5/group. (e) Quantification of flux (individual mice per group) from OV90(eGFP/ffluc) tumor-bearing mice treated i.p. with CD19-CAR T cells (n=5/group), CD19-CAR/mbIL12 T cells (n=5/group), TAG72-CAR T cells (n=9-10/group) and TAG72-CAR/mbIL12 T cells (n=9-10/group). Combined data are from two independent studies. (f) Quantification of TAG72-CAR T cells per uL of peritoneal ascites (left) at weeks 2 and 4 post-treatment. n=2 per group. Quantification of TAG72-CAR T cells per uL of peripheral blood (right) at weeks 1, 2, and 4 post-treatment. n=5/group.

FIG. 12 Locoregional intraperitoneal delivery of TAG72-CAR/mbIL12 T cells eradicate subcutaneous tumors in dual tumor-bearing mice. (a) Schematic for subcutaneous (s.c.) and i.p. OV90 dual tumor model and treatment. (b) Bioluminescent flux imaging of dual tumor-bearing mice treated i.p. with TAG72-CAR or TAG72-CAR/mbIL12 T cells. (c) Quantification of flux (individual mice per group) from mice treated i.p. with TAG72-CAR T cells (n=8/group) and TAG72-CAR/mbIL12 T cells (n=8/group). (d) Quantification of subcutaneous tumor volume (individual mice per group) from mice treated i.p. with TAG72-CAR T cells. (e) Quantification of TAG72-CAR T cells per uL of peripheral blood at days 7, 14, 21, and 28 post-treatment. n=5/group. (f) Quantification of TAG72-CAR T cells per uL of peritoneal ascites at week 2 post-treatment. n=2/group. (g) Immunohistochemistry analysis of CD3+ T cells in s.c. tumors at day #post-treatment.

FIG. 13 mbIL-12 expressing TAG72-CAR T cells do not expand and survive in the absence of exogenous cytokines. (a-b) Quantification of T cell count (left) and percentage of viable cells (right) during ex vivo culture in the absence (a) or presence (b) of exogenous cytokines as described in Materials and Methods.

FIG. 14 mbIL-12 signaling improves in vitro functionality of PSCA- and HER2-CAR T cells. (a-f) Quantification of tumor cells (left), T cells (middle), and IFNγ levels in supernatant (right) for PSCA-CAR T cells (a-c) and HER2-CAR T cells (d-f), following co-culture with antigen-positive targets (PC3-PSCA or 468-HER2) at an E:T ratio of 1:10 for 6 days. (g) HER2-CAR T cells with or without mbIL12 were co-cultured with 468-HER2 cells (E:T=1:10) and rechallenged with 468-HER2 cells every three days. Remaining viable tumor cells (g) and T cells (h) were quantified by flow cytometry prior to every rechallenge and two or three days after the last rechallenge with 468-HER2 cells. Percentage of (i-k) CD4⁺ (i), CD8⁺ (j), and CD62L⁺ CCR7⁺ T cells were measured by flow cytometry from the rechallenge assay.

FIG. 15 TAG72-dCH2(CD28tm)BBz CAR shows comparable pSLP76 expression, but damped pPLCy and pERK signaling compared to an otherwise identical TAG72 CAR with a CD28 costimulatory domain. (a-c)

FIG. 16 mbIL-12 improves in vivo regional brain and systemic bone metastatic disease targeting by HER2 CAR. Locoregional intracerebroventricular delivery of HER2-CAR/mbIL12 T cells reduces tumor burden and increases regional and systemic CAR T cell persistence in vivo. (a) Schematic for intratibial (i.ti.) and intracranial (i.e.) BBM1 dual tumor model and treatment. (b) Bioluminescent flux imaging of dual tumor-bearing mice left untreated (no tx), or treated by intracerebroventricular (i.c.v.) injection of HER2-CAR or HER2-CAR/mbIL12 T cells. (c) Quantification of brain (top) or bone (bottom) flux from individual mice treated i.c.v. with HER2-CAR T cells (n>10/group), HER2-CAR/mbIL12 T cells (n>10/group), or no tx (n=4/group). (d-e) Representative flow cytometric analysis (d) and quantification (e) of HER2-CAR T cells per uL of blood at weeks 1, 2, and 5 post-treatment. n>3/group. (f) Immunohistochemistry of CD3+ T cells in i.ti. and i.e. tumors at day 7 post-treatment. (g-h) Increases in central memory CAR T cells in peripheral blood of HER2-CAR/mbIL12 T cell treated mice. (g) Flow cytometric analysis of the frequency of HER2-CAR and HER2-CAR/mbIL12 T cells in the peripheral blood (h) Quantification of CAR T cells per uL of peripheral blood at 35 days post-treatment. n=5-7/group. (i) Quantification of brain (top) or bone (bottom) flux from individual mice treated i.c.v. with HER2-CAR T cells (n>10/group), HER2-CAR/mbIL12 T cells (n>10/group). (j) Quantification of HER2-CAR T cells per uL of blood at weeks 1 and 2 weeks following treatment. n>3/group.

FIG. 17 mbIL-12 cytokine signaling in CAR T cells appears safer than recombinant IL-12 injection and demonstrates potent antitumor activity. (a-c) Locoregional intraperitoneal delivery of TAG72-CAR/mbIL12 T cells safely and effectively target ovarian cancer peritoneal metastasis in an immune-competent syngeneic mouse model. (a) Average tumor flux (left) and percent weight change (right) in indicated T cell treatments relative to pre-treatment weight (n>7/group). (b) Lymphocytes, monocytes, and neutrophils percentages from complete blood count analysis collected at 7 days post treatment (top), and quantification of serum levels of ALT (bottom) from mice at 6 days following treatment (n≥4/group). (c) ELISA quantification of IFNγ (left) and IL-12 (right) cytokines in mouse serum at day 18 and 20 (day 4 and 6 post-treatment respectively; n≥5/group).

FIG. 18 In vitro analysis of CAR T cells with varying extracellular spacer, transmembrane and costimulatory domains. In vitro T cell proliferation in fold change compared to UTD (a) expression of CD137 (b) and PD-1 (c) by flow cytometry, and IL-2 production (d) by ELISA (c), of CAR T cells against tumor targets (TAG72-OVCAR8; TAG72+ OVCAR3, OV90, and OVCAR8-sTn) after 24 hr (for ELISA) or 72 hr of co-culture at an effector:target (E:T) ratio of 1:4, to support CAR T cell functional data in FIG. 1c-d. Statistical analysis reflects comparisons between TAG72-dCH2(28tm)28z and TAG72-dCH2(28tm)BBz. (e) HER2-CAR T cell killing of HER2⁺ SKOV3 cells measured by xCELLigence over 5 days (E:T=1:20). Remaining T cells (f), along with expression of CD137 (g) and PD-1 (h) by flow cytometry.

FIG. 19 Membrane-bound IL-12 engineered TAG72-CAR T cells induce higher IFNγ, T cell expansion, and anti-tumor activity in vitro. (a-b) Tumor cell killing of OV90 cells by TAG72-CAR T cells (E:T=1:20) with addition of varying concentrations of anti-IFNγR1 blocking antibody, isotype control, and recombinant human IL-12 cytokine measured by xCELLigence over 10 days (a-b). Locoregional intraperitoneal delivery of TAG72-CAR/mbIL12 T cells reduces tumor burden and increases regional and systemic CAR T cell persistence in vivo. The i.p. OV90(eGFP/ffluc) tumor-bearing mice treated 1018 i.p. with CD19-CAR, CD19-CAR/mbIL12, TAG72-CAR or TAG72-CAR/mbIL12 T cells. (c) Flow cytometric analysis of TAG72-CAR T cells per uL of peritoneal ascites. (d) Quantification of TAG72-CAR T cells per uL of peritoneal ascites (left) at weeks 2 and 4 post-treatment. n=2 per group. Quantification of TAG72-CAR T cells per uL of peripheral blood (right) at weeks 1, 2, and 4 post-treatment. n=5/group.

FIG. 20 Generation and functional characterization in vitro of murine TAG72-CAR expressing murine mbIL12. (a) Illustration of dual expression of murine TAG72-CAR and murine mbIL12 on mouse T cell surface. (b) Flow cytometric analysis of surface TAG72-CAR expression as detected by expression of truncated murine CD19t (left) and surface murine mbIL12 as detected by anti-mIL12 PE conjugated antibody. (c) Flow cytometric analysis of murine CD4 and CD8 ratios in TAG72-CAR and TAG72-CAR/mbIL12. (d-g) Functional characterization of 72-hour long term co-culture killing assay tumor killing (d) against ID80-mSTn tumor cells at 1:1 E:T (e), T cell counts (f), CAR T cell CD137 (g), and post co-culture CD8 and CD4 ratios (h) in TAG72-CAR vs. TAG72-CAR/mbIL12 T cells.

FIG. 21 Locoregional intraperitoneal delivery of TAG72-CAR/mbIL12 T cells safely and effectively target ovarian cancer peritoneal metastasis in an immune-competent syngeneic mouse model. (a) Schematic for intraperitoneal (i.p.) ID8-mSTn tumor model and treatment. (b) Bioluminescence flux imaging of tumor-bearing mice, treated by intraperitoneal (i.p.) injection of indicated T cells. (c) Average tumor flux and (d) percent weight change in indicated T cell treatments relative to pre-treatment weight (n>7/group). (e) Spleen photographs (n=3/group) and (f) CD3 IHC in livers harvested at 7 days post treatment. (g) Quantification of serum levels of ALT (left) and AST (right) from mice at 6 days post treatment (n≥4/group). (h) Percent lymphocytes, monocytes, and neutrophils from complete blood count analysis collected at 7 days post treatment. ELISA quantification of IFNγ (i) and IL-12 (j) cytokines in mouse serum at day 18 and 20 (day 4 and 6 post treatment respectively) post tumor injection (n≥4/group). (k) Systemic effects of slL-12 and TAG72-CAR/mbIL12 T cells. H&E spleen and liver collected from i.p. ID8-mSTn tumor-bearing mice treated with indicated T cells.

FIG. 22 TAG72-CAR/mbIL12 T cell therapy induces TME modifications in ovarian cancer peritoneal metastases in an immune-competent syngeneic mouse model. (a) H&E and CD3 IHC in solid tumor masses collected from the upper omental region of i.p. ID8-mSTn tumor-bearing mice treated with indicated T cells. (b) Flow cytometric analysis of tumor cells (CD3-CD45-double negative) in peritoneal ascites. (c) Quantification of tumor cells (CD3-CD45-double negative) and (d) immune subsets (CD45+, CD3+, CD11 b+ and NK+) as cells/mL in peritoneal ascites. (e) Flow cytometric analysis of percent CAR T cells (CD3+CD19t+) and (f) quantification counts of CD4+ and CD8+ CAR T cells/mL in peritoneal ascites. (g) Quantification of mean fluorescent intensity (MFI) of CD137+ in CAR T cells in peritoneal ascites. (h) Quantification of percent CD62L+CD44+(Tcm) in both CAR+ and CAR− T cells in peritoneal ascites. (i) Quantification of myeloid cell counts (Ly6G+, Ly6C+, Ly6G−/C− double negative tumor-associated macrophages (TAM) and CD11c+CD103+ dendritic cells (DC) as cells/mL in peritoneal ascites gated from total CD11b+1073 cells. Flow cytometric analysis of percent (j) and quantification of MFI (k) on CD103+MHC Class II+ double positive DC in peritoneal ascites. All analyses represent data collected from ascites of ID8-mSTn tumor-bearing mice at 7 days post treatment, n=3/group. (1) Flow cytometry gating strategy. (m) Total CD4+ and CD8+ T cells mL of peritoneal ascites.

FIG. 23 In vitro safety of TAG72-CAR/mbIL12 T cells against normal human cell lines. (a) Flow cytometric analysis of TAG72 expression on the cell surface of TAG72-negative (OVCAR8) tumor cells, TAG72-positive (OV90) tumor cells, and indicated patient-derived gastric (GAS1) and ovarian (OAS3 and 4) peritoneal ascites. (b-c) TAG72-CAR T cells with or without mbIL12 were co-cultured with tumor and normal cell at 1:4 E:T ratio for 48 hours. (b) Quantification of CD137 expression in CAR T cells was measured by flow cytometry. (c) IFNγ levels in supernatant were quantified by ELISA. (d) IFNγ signaling drives anti-tumor activity in TAG72-CAR/mbIL12 T cells in vitro. Tumor cell killing of OV90 cells by TAG72-CAR/mbIL12 T cells (E:T=1:20) with addition of varying concentrations of anti-IFNγR1 blocking antibody or isotype by xCELLigence over 10 days.

### DETAILED DESCRIPTION

### Membrane Bound IL-12

The membrane bound IL-12 include a human CD28 transmembrane domain that is important for providing membrane bound IL-12 with ability to improve the function of CAR T cells. Thus, mb(28)IL-12 includes, from amino to carboxy terminus: mature human IL-12 beta (p40) subunit, an optional first peptide linker, mature human IL-12 alpha (p35) subunit, an optional second peptide linker, human CD28 transmembrane domain. In some embodiments, all or a portion of the cytoplasmic domain of human CD28 follows the transmembrane domain. A signal sequence can precede the human II-12 beta subunit.

The p40 subunit portion comprises or consists of the sequence:

(SEQ ID NO: 1)
IWELKKDVYVVELDWYPDAPGEMVVLTCDTPEEDGITWTLDQSSE
VLGSGKTLTIQVKEFGDAGQYTCHKGGEVLSHSLLLLHKKEDGIW
STDILKDQKEPKNKTFLRCEAKNYSGRFTCWWLTTISTDLTFSVK
SSRGSSDPQGVTCGAATLSAERVRGDNKEYEYSVECQEDSACPAA
EESLPIEVMVDAVHKLKYENYTSSFFIRDIIKPDPPKNLQLKPLK
NSRQVEVSWEYPDTWSTPHSYFSLTFCVQVQGKSKREKKDRVFTD
KTSATVICRKNASISVRAQDRYYSSSWSEWASVPCS

The first peptide linker comprises or consists of, for example, the sequence

(SEQ ID NO: 4)
VPGVGVPGVG

The p35 subunit portion comprises or consists of the sequence:

(SEQ ID NO: 2)
ARNLPVATPDPGMFPCLHHSQNLLRAVSNMLQKARQTLEFYPCTS
EEIDHEDITKDKTSTVEACLPLELTKNESCLNSRETSFITNGSCL
ASRKTSFMMALCLSSIYEDSKMYQVEFKTMNAKLLMDPKRQIFLD
QNMLAVIDELMQALNFNSETVPQKSSLEEPDFYKTKIKLCILLHA
FRIRAVTIDRVMSYLNAS

The p35 subunit portion comprises or consists of the sequence:

(SEQ ID NO: 3)
ARNLPVATPDPGMFPCLHHSQNLLRAVSNMLQKARQTLEFYPCTSEEID
HEDITKDKTSTVEACLPLELTKNESCLNSRETSFITNGSCLASRKTSFM
MALCLSSIYEDLKMYQVEFKTMNAKLLMDPKRQIFLDQNMLAVIDELMQ
ALNFNSETVPQKSSLEEPDFYKTKIKLCILLHAFRIRAVTIDRVMSYLN
AS

The second peptide linker comprises or consists of, for example, the sequence:

GGG

The human CD28 transmembrane domain comprises or consists of the sequence:

(SEQ ID NO: 16)
FWVLVVVGGVLACYSLLVTVAFIIFWV.

The human CD28 cytoplasmic domain portion comprises or consists of the sequence:

(SEQ ID NO: 5)
RSKR

A human CD28 transmembrane domain that includes a portion of the human CD28 cytoplasmic domain can comprise or consist of the sequence:

(SEQ ID NO: 76)
FWVLVVVGGVLACYSLLVTVAFIIFWVRSKR.

Mature mb(28)IL-12 can comprise or consist of the sequence:

(SEQ ID NO: 6)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFCVQVQGKSKREKKDRVFTDKTSATVICRKNASISVRAQDRYYS
SSWSEWASVPCSVPGVGVPGVGARNLPVATPDPGMFPCLHHSQNLLRAV
SNMLQKARQTLEFYPCTSEEIDHEDITKDKTSTVEACLPLELTKNESCL
NSRETSFITNGSCLASRKTSFMMALCLSSIYEDSKMYQVEFKTMNAKLL
MDPKRQIFLDQNMLAVIDELMQALNFNSETVPQKSSLEEPDFYKTKIKL
CILLHAFRIRAVTIDRVMSYLNASGGGFWVLVVVGGVLACYSLLVTVAF
IIFWVRSKR

Mature mb(28)IL-12 can comprise or consist of the sequence:

(SEQ ID NO: 7)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFCVQVQGKSKREKKDRVFTDKTSATVICRKNASISVRAQDRYYS
SSWSEWASVPCSVPGVGVPGVGARNLPVATPDPGMFPCLHHSQNLLRAV
SNMLQKARQTLEFYPCTSEEIDHEDITKDKTSTVEACLPLELTKNESCL
NSRETSFITNGSCLASRKTSFMMALCLSSIYEDLKMYQVEFKTMNAKLL
MDPKRQIFLDQNMLAVIDELMQALNFNSETVPQKSSLEEPDFYKTKIKL
CILLHAFRIRAVTIDRVMSYLNASGGGFWVLVVVGGVLACYSLLVTVAF
IIFWVRSKR

In this sequence the first and second peptide linker sequences are underlined, the second peptide; the human CD28 transmembrane domain sequence is in bold and the portion of human CD28 cytoplasmic domain sequence is in italics.

The mature sequence can be preceded by a signal sequence suitable for directing secretion to the surface of a human cell.

For example, the signal sequence can comprise or consist of the sequence:

(SEQ ID NO: 10)
MCHQQLVISWFSLVFLASPLVA

In one example, mb(CD28)IL-12 with a signal sequence can comprise or consist of the sequence:

(SEQ ID NO: 8)
MCHQQLVISWFSLVFLASPLVAIWELKKDVYVVELDWYPDAPGEMVVLT
CDTPEEDGITWTLDQSSEVLGSGKTLTIQVKEFGDAGQYTCHKGGEVLS
HSLLLLHKKEDGIWSTDILKDQKEPKNKTFLRCEAKNYSGRFTCWWLTT
ISTDLTFSVKSSRGSSDPQGVTCGAATLSAERVRGDNKEYEYSVECQED
SACPAAEESLPIEVMVDAVHKLKYENYTSSFFIRDIIKPDPPKNLQLKP
LKNSRQVEVSWEYPDTWSTPHSYFSLTFCVQVQGKSKREKKDRVFTDKT
SATVICRKNASISVRAQDRYYSSSWSEWASVPCSVPGVGVPGVGARNLP
VATPDPGMFPCLHHSQNLLRAVSNMLQKARQTLEFYPCTSEEIDHEDIT
KDKTSTVEACLPLELTKNESCLNSRETSFITNGSCLASRKTSFMMALCL
SSIYEDSKMYQVEFKTMNAKLLMDPKRQIFLDQNMLAVIDELMQALNFN
SETVPQKSSLEEPDFYKTKIKLCILLHAFRIRAVTIDRVMSYLNASGGG
FWVLVVVGGVLACYSLLVTVAFIIFWVRSKR

In this sequence the signal sequence is underlined.

In one example, mb(CD28)IL-12 with a signal sequence can comprise or consist of the sequence:

(SEQ ID NO: 9)
MCHQQLVISWFSLVFLASPLVAIWELKKDVYVVELDWYPDAPGEMVVLT
CDTPEEDGITWTLDQSSEVLGSGKTLTIQVKEFGDAGQYTCHKGGEVLS
HSLLLLHKKEDGIWSTDILKDQKEPKNKTFLRCEAKNYSGRFTCWWLTT
ISTDLTFSVKSSRGSSDPQGVTCGAATLSAERVRGDNKEYEYSVECQED
SACPAAEESLPIEVMVDAVHKLKYENYTSSFFIRDIIKPDPPKNLQLKP
LKNSRQVEVSWEYPDTWSTPHSYFSLTFCVQVQGKSKREKKDRVFTDKT
SATVICRKNASISVRAQDRYYSSSWSEWASVPCSVPGVGVPGVGARNLP
VATPDPGMFPCLHHSQNLLRAVSNMLQKARQTLEFYPCTSEEIDHEDIT
KDKTSTVEACLPLELTKNESCLNSRETSFITNGSCLASRKTSFMMALCL
SSIYEDLKMYQVEFKTMNAKLLMDPKRQIFLDQNMLAVIDELMQALNFN
SETVPQKSSLEEPDFYKTKIKLCILLHAFRIRAVTIDRVMSYLNASGGG
FWVLVVVGGVLACYSLLVTVAFIIFWVRSKR

In this sequence the signal sequence is underlined.

In other cases, the signal sequence can comprise or consist of

(SEQ ID NO: 11)
MLLLVTSLLLCELPHPAFLLIP.

In one example, mbIL-12 with a signal sequence can comprise or consist of the sequence:

(SEQ ID NO: 44)
MLLLVTSLLLCELPHPAFLLIPIWELKKDVYVVELDWYPDAPGEMVVLT
CDTPEEDGITWTLDQSSEVLGSGKTLTIQVKEFGDAGQYTCHKGGEVLS
HSLLLLHKKEDGIWSTDILKDQKEPKNKTFLRCEAKNYSGRFTCWWLTT
ISTDLTFSVKSSRGSSDPQGVTCGAATLSAERVRGDNKEYEYSVECQED
SACPAAEESLPIEVMVDAVHKLKYENYTSSFFIRDIIKPDPPKNLQLKP
LKNSRQVEVSWEYPDTWSTPHSYFSLTFCVQVQGKSKREKKDRVFTDKT
SATVICRKNASISVRAQDRYYSSSWSEWASVPCSVPGVGVPGVGARNLP
VATPDPGMFPCLHHSQNLLRAVSNMLQKARQTLEFYPCTSEEIDHEDIT
KDKTSTVEACLPLELTKNESCLNSRETSFITNGSCLASRKTSFMMALCL
SSIYEDSKMYQVEFKTMNAKLLMDPKRQIFLDQNMLAVIDELMQALNFN
SETVPQKSSLEEPDFYKTKIKLCILLHAFRIRAVTIDRVMSYLNASGGG
FWVLVVVGGVLACYSLLVTVAFIIFWVRSKR

In one example, mbIL-12 with a signal sequence can comprise or consist of the sequence:

(SEQ ID NO: 49)
MLLLVTSLLLCELPHPAFLLIPIWELKKDVYVVELDWYPDAPGEMVVLT
CDTPEEDGITWTLDQSSEVLGSGKTLTIQVKEFGDAGQYTCHKGGEVLS
HSLLLLHKKEDGIWSTDILKDQKEPKNKTFLRCEAKNYSGRFTCWWLTT
ISTDLTFSVKSSRGSSDPQGVTCGAATLSAERVRGDNKEYEYSVECQED
SACPAAEESLPIEVMVDAVHKLKYENYTSSFFIRDIIKPDPPKNLQLKP
LKNSRQVEVSWEYPDTWSTPHSYFSLTFCVQVQGKSKREKKDRVFTDKT
SATVICRKNASISVRAQDRYYSSSWSEWASVPCSVPGVGVPGVGARNLP
VATPDPGMFPCLHHSQNLLRAVSNMLQKARQTLEFYPCTSEEIDHEDIT
KDKTSTVEACLPLELTKNESCLNSRETSFITNGSCLASRKTSFMMALCL
SSIYEDLKMYQVEFKTMNAKLLMDPKRQIFLDQNMLAVIDELMQALNFN
SETVPQKSSLEEPDFYKTKIKLCILLHAFRIRAVTIDRVMSYLNASGGG
FWVLVVVGGVLACYSLLVTVAFIIFWVRSKR
In some case, the sequence
(SEQ ID NO: 16)
FWVLVVVGGVLACYSLLVTVAFIIFWV can be replaced with
(SEQ ID NO: 17)
MFWVLVVVGGVLACYSLLVTVAFIIFWV
or
(SEQ ID NO: 76)
FWVLVVVGGVLACYSLLVTVAFIIFWVRSKR.

The first and second linker peptide linker can be have any suitable sequence. For example, the first peptide linker can consist of: 3-24 amino acids, 3-20 amino acids, 3-15 amino acids, 3-10 amino acids (e.g., 3, 4, 5, 6, 7, 8, 9, 10, 11, 12, 13, 14, 15, 16, 17, 18, 19, or 20 amino acids. Preferably, none of the amino acids are proline. For example, the second peptide linker can consist of: 3-24 amino acids, 3-20 amino acids, 3-15 amino acids, 3-10 amino acids (e.g., 3, 4, 5, 6, 7, 8, 9, 10, 11, 12, 13, 14, 15, 16, 17, 18, 19, or 20 amino acids. Preferably, none of the amino acids are proline. Suitable second peptide linkers include glycines and or a mixture of glycines and serines (e.g., GGGGSGGGGS GGGGSGGGGS (SEQ ID NO: 57), GGGG (SEQ ID NO: 58), GGGGS (SEQ ID NO: 14), GGGSGG (SEQ ID NO: 59) and GGGSGGGS (SEQ ID NO: 60).

The portion of the human cytoplasmic domain when present preferably lacks signaling activity.

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

### Chimeric Antigen Receptors

A chimeric antigen receptor (CAR) refers to an artificial immune cell receptor that is engineered to recognize and bind to a surface antigen. A T cell that expresses a CAR polypeptide is referred to as a CAR T cell. CARs have the ability to redirect T-cell specificity and reactivity toward a selected target in a non-MHC-restricted manner. The non-MHC-restricted antigen recognition gives CAR T cells the ability to recognize an antigen independent of antigen processing, thereby bypassing a major mechanism of tumor escape. A CAR can also be expressed by other immune effector cells, including but not limited to natural killer CAR (“NK CAR”) and directed NK cell killing to cells expressing the target of the CAR.

There are various generations of CARs, each of which contains different components. First generation CARs join an antibody-derived scFv to the CD3ζ (intracellular signaling domain of the T cell receptor through a spacer region (also called a hinge domain) and a transmembrane domain. Second generation CARs incorporate an additional co-stimulatory domain (e.g., CD28, 4-BB, or ICOS) to supply a co-stimulatory signal. Third generation CARs contain two co-stimulatory domains (e.g., a combination of CD27, CD28, 4-1 BB, ICOS, or OX40) fused with the TCR CD3ζ (chain.

### (a) Extracellular Binding Domain

The CAR described herein are fusion proteins comprising an extracellular binding domain that recognizes a tumor antigen, e.g., a solid tumor antigen. This extracellular domain is often a single chain fragment (scFv) of an antibody or other antibody fragment, but it can also be a ligand that binds to a cell receptor. The binding domain is followed by: a spacer, a transmembrane domain, at least one co-stimulatory domain and an intracellular domain comprising a signaling domain of the T cell receptor (TCR) complex (e.g., CD3ζ). A CAR is often fused to a signal peptide at the N-terminus for surface expression.

Where the binding domain is an scFv, there is a heavy chain variable region and a light chain variable region, which can be in an order and are joined together via a flexible linker of, e.g., 5-25 amino acids. In some embodiments, a useful flexible linker is 1, 2, 3, 4, 5, 6, 7, 8, 9, or 10 repeats of the sequence GGGS (SEQ ID NO: 13). In some embodiments, a useful flexible linker is 1, 2, 3, 4, 5, 6, 7, 8, 9, or 10 repeats of the sequence GGGGS (SEQ ID NO: 14) or SEQ ID NO:13. In some embodiments, the light chain variable domain is amino terminal to the heavy chain variable domain in other cases it is carboxy terminal to the heavy chain variable domain. In some cases the linker comprises the sequence

(SEQ ID NO: 12)
SSGGGGSGGGGSGGGGS.

### (b) Transmembrane Domain

The CAR polypeptides disclosed herein can contain a transmembrane domain, which can be a hydrophobic alpha helix that spans the membrane. As used herein, a transmembrane domain refers to any protein structure that is thermodynamically stable in a cell membrane, preferably a eukaryotic cell membrane.

The transmembrane domain of a CAR as provided herein can be a CD28 transmembrane domain having the sequence:

(SEQ ID NO: 16)
FWVLVVVGGVLACYSLLVTVAFIIFWV.

Other transmembrane domains can be used including those shown below.

TABLE 1
Examples of Transmembrane Domains

| Name | Accession | Length | Sequence |
|---|---|---|---|
| CD3z | J04132.1 | 21 aa | LCYLLDGILFIYGVILTALFL (SEQ ID NO: 15) |
| CD28 | NM_006139 | 27 aa | FWVLVVVGGVLACYSLLVTVAFIIFWV (SEQ ID NO: 16) |
| CD28(M) | NM_006139 | 28 aa | MFWVLVVVGGVLACYSLLVTVAFIIFWV (SEQ ID NO: 17) |
| CD4 | M35160 | 22 aa | MALIVLGGVAGLLLFIGLGIFF (SEQ ID NO: 18) |
| CD8tm | NM_001768 | 21 aa | IYIWAPLAGTCGVLLLSLVIT (SEQ ID NO: 19) |
| CD8tm2 | NM_001768 | 23 aa | IYIWAPLAGTCGVLLLSLVITLY (SEQ ID NO: 20) |
| CD8tm3 | NM_001768 | 24 aa | IYIWAPLAGTCGVLLLSLVITLYC (SEQ ID NO: 21) |
| 4-1BB | NM_001561 | 27 aa | IISFFLALTSTALLFLLFFLTLRFSVV (SEQ ID NO: 22) |
| NKG2D | NM_007360 | 21 aa | PFFFCCFIAVAMGIRFIIMVA (SEQ ID NO: 23) |

### (c) Spacer Region

The CAR or polypeptide described herein can include a spacer region located between the targeting domain (i.e., a HER2 or TAG-72 targeted scFv or variant thereof) and the transmembrane domain. The spacer region can function to provide flexibility to the CAR, or domains thereof, or to prevent steric hindrance of the CAR, or domains thereof. A variety of different spacers can be used. Some of them include at least portion of a human Fc region, for example a hinge portion of a human Fc region or a CH3 domain or variants thereof. Table 2 below provides various spacers that can be used in the CARs described herein.

TABLE 2
Examples of Spacers

| Name | Length | Sequence |
|---|---|---|
| a3 | 3 aa | AAA |
| linker | 10 aa | GGGSSGGGSG (SEQ ID NO: 24) |
| IgG4 hinge (S→P) | 12 aa | ESKYGPPCPPCP (SEQ ID NO: 25) |
| (S228P) | | |
| IgG4 hinge | 12 aa | ESKYGPPCPSCP (SEQ ID NO: 26) |
| IgG4 hinge | 22 aa | ESKYGPPCPPCPGGGSSGGGSG (SEQ ID NO: 27) |
| (S228P) + linker | | |
| CD28 hinge | 39 aa | IEVMYPPPYLDNEKSNGTIIHVKGKHLCPSPLFPGPSK |
| | | P (SEQ ID NO: 28) |
| CD8 hinge-48aa | 48 aa | AKPTTTPAPRPPTPAPTIASQPLSLRPEACRPAAGGAV |
| | | HTRGLDFACD (SEQ ID NO: 29) |
| CD8 hinge-45aa | 45 aa | TTTPAPRPPTPAPTIASQPLSLRPEACRPAAGGAVHTR |
| | | GLDFACD (SEQ ID NO: 30) |
| IgG4(HL-CH3) | 129 aa | ESKYGPPCPPCPGGGSSGGGSGGQPREPQVYTLPPSQE |
| Also called IgG4 | | EMTKNQVSLTCLVKGFYPSDIAVEWESNGQPENNYKTT |
| (HL-ΔCH2) | | PPVLDSDGSFFLYSRLTVDKSRWQEGNVFSCSVMHEAL |
| (includes S228P | | HNHYTQKSLSLSLGK (SEQ ID NO: 31) |
| in hinge) | | |
| IgG4(L235E, | 229 aa | ESKYGPPCPSCPAPEFEGGPSVFLFPPKPKDTLMISRT |
| N297Q) | | PEVTCVVVDVSQEDPEVQFNWYVDGVEVHNAKTKPREE |
| | | QFQSTYRVVSVLTVLHQDWLNGKEYKCKVSNKGLPSSI |
| | | EKTISKAKGQPREPQVYTLPPSQEEMTKNQVSLTCLVK |
| | | GFYPSDIAVEWESNGQPENNYKTTPPVLDSDGSFFLYS |
| | | RLTVDKSRWQEGNVFSCSVMHEALHNHYTQKSLSLSLG |
| | | K (SEQ ID NO: 32) |
| IgG4(S228P, | 229 aa | ESKYGPPCPPCPAPEFEGGPSVFLFPPKPKDTLMISRT |
| L235E, N297Q) | | PEVTCVVVDVSQEDPEVQFNWYVDGVEVHNAKTKPREE |
| | | QFQSTYRVVSVLTVLHQDWLNGKEYKCKVSNKGLPSSI |
| | | EKTISKAKGQPREPQVYTLPPSQEEMTKNQVSLTCLVK |
| | | GFYPSDIAVEWESNGQPENNYKTTPPVLDSDGSFFLYS |
| | | RLTVDKSRWQEGNVFSCSVMHEALHNHYTQKSLSLSLG |
| | | K (SEQ ID NO: 33) |
| IgG4(CH3) | 107 aa | GQPREPQVYTLPPSQEEMTKNQVSLTCLVKGFYPSDIA |
| Also called IgG4 | | VEWESNGQPENNYKTTPPVLDSDGSFFLYSRLTVDKSR |
| (ΔCH2) | | WQEGNVFSCSVMHEALHNHYTQKSLSLSLGK (SEQ |
| | | ID NO: 34) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain (called CH3 or ΔCH2) or both a CH3 domain and a CH2 domain. The immunoglobulin derived sequences can include one or more amino acid modifications, for example, 1, 2, 3, 4 or 5 substitutions, e.g., substitutions that reduce off-target binding.

The hinge/linker region can also comprise an IgG4 hinge region having the sequence ESKYGPPCPSCP (SEQ ID NO: 26) or ESKYGPPCPPCP (SEQ ID NO: 25). The hinge/linger region can also comprise the sequence ESKYGPPCPPCP (SEQ ID NO: 25) followed by the linker sequence GGGSSGGGSG (SEQ ID NO: 24) followed by IgG4 CH3 sequence:

(SEQ ID NO: 34)
GQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKS
LSLSLGK.

Thus, the entire linker/spacer region can comprise the sequence: ESKYGPPCPPCPGGGSSGGGSGGQPREPQVYTLPPSQEEMTKNQVSLTCLVKGFYPSDI AVEWESNGQPENNYKTTPPVLDSDGSFFLYSRLTVDKSRWQEGNVFSCSVMHEALHNHY TQKSLSLSLGK (SEQ ID NO: 31). In some cases, the spacer has 1, 2, 3, 4, or 5 single amino acid changes (e.g., conservative changes) compared to SEQ ID NO: 31. In some cases, the IgG4 Fc hinge/linker region that is mutated at two positions (e.g., L235E and N297Q) in a manner that reduces binding by Fc receptors (FcRs).

### (d) Intracellular Signaling Domains

Any of the CAR constructs described herein contain one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three immunoreceptor tyrosine-based activation motifs (ITAMs), which transmit an activation signal to the T cell after the T cell is engaged with a cognate antigen. In some cases, CD3ζ provides a primary T cell activation signal but not a fully competent activation signal, which requires a co-stimulatory signal.

Accordingly, in some examples, the CAR polypeptides disclosed herein may further comprise one or more co-stimulatory signaling domains in addition to CD3 ζ. For example, the co-stimulatory domain CD28 and/or 4-1 BB can be used to transmit a proliferative/survival signal together with the primary signaling mediated by CD3ζ.

The co-stimulatory domain(s) are located between the transmembrane domain and the CD3ζ (signaling domain. Table 3 includes examples of suitable co-stimulatory domains together with the sequence of the CD3ζ signaling domain.

TABLE 3
CD3ζ Domain and Examples of Co-stimulatory Domains

| Name | Accession | Length | Sequence |
|---|---|---|---|
| CD3ζ | J04132.1 | 113 aa | RVKFSRSADAPAYQQGQNQLYNELNLGRREEYDVLDKRR |
| | | | GRDPEMGGKPRRKNPQEGLYNELQKDKMAEAYSEIGMKG |
| | | | ERRRGKGHDGLYQGLSTATKDTYDALHMQALPPR (SEQ |
| | | | ID NO: 35) |
| | | | ITAMS 1-3 underlined |
| CD3ζ | | 113 aa | RVKFSRSADAPAYQQGQNQLYNELNLGRREEYDVLDKRR |
| variant | | | GRDPEMGGKPRRKNPQEGLFNELQKDKMAEAFSEIGMKG |
| | | | ERRRGKGHDGLFQGLSTATKDTFDALHMQALPPR (SEQ |
| | | | ID NO: 50) |
| CD3ζ | | 113 aa | RVKFSRSADAPAYQQGQNQLYNELNLGRREEYDVLDKRR |
| variant | | | GRDPEMGGKPRRKNPQEGLFNELQKDKMAEAFSEIGMKG |
| | | | ERRRGKGHDGLYQGLSTATKDTYDALHMQALPPR (SEQ |
| | | | ID NO: 51) |
| CD3ζ | | 113 aa | RVKFSRSADAPAYQQGQNQLYNELNLGRREEYDVLDKRR |
| variant | | | GRDPEMGGKPRRKNPQEGLYNELQKDKMAEAYSEIGMKG |
| | | | ERRRGKGHDGLFQGLSTATKDTFDALHMQALPPR (SEQ |
| | | | ID NO: 52) |
| CD3ζ | | 113 aa | RVKFSRSADAPAYQQGQNQLYNELNLGRREEYDVLDKRR |
| variant | | | GRDPEMGGKPRRKNPQEGLYNELQKDKMAEAFSEIGMKG |
| | | | ERRRGKGHDGLFQGLSTATKDTFDALHMQALPPR (SEQ |
| | | | ID NO: 53) |
| CD3ζ | | 113 aa | RVKFSRSADAPAYQQGQNQLYNELNLGRREEYDVLDKRR |
| variant | | | GRDPEMGGKPRRKNPQEGLFNELQKDKMAEAYSEIGMKG |
| | | | ERRRGKGHDGLFQGLSTATKDTFDALHMQALPPR (SEQ |
| | | | ID NO: 54) |
| CD3ζ | | 113 aa | RVKFSRSADAPAYQQGQNQLYNELNLGRREEYDVLDKRR |
| variant | | | GRDPEMGGKPRRKNPQEGLFNELQKDKMAEAFSEIGMKG |
| | | | ERRRGKGHDGLYQGLSTATKDTFDALHMQALPPR (SEQ |
| | | | ID NO: 55) |
| CD3ζ | | 113 aa | RVKFSRSADAPAYQQGQNQLYNELNLGRREEYDVLDKRR |
| variant | | | GRDPEMGGKPRRKNPQEGLFNELQKDKMAEAFSEIGMKG |
| | | | ERRRGKGHDGLFQGLSTATKDTYDALHMQALPPR (SEQ |
| | | | ID NO: 56) |
| CD28 | NM_006139 | 42 aa | RSKRSRLLHSDYMNMTPRRPGPTRKHYQPYAPPRDFAAY |
| | | | RS (SEQ ID NO: 36) |
| CD28gg* | NM_006139 | 42 aa | RSKRSRGGHSDYMNMTPRRPGPTRKHYQPYAPPRDFAAY |
| | | | RS (SEQ ID NO: 37) |
| 41BB | NM_001561 | 42 aa | KRGRKKLLYIFKQPFMRPVQTTQEEDGCSCRFPEEEEGG |
| | | | CEL (SEQ ID NO: 38) |
| OX40 | NM_003327 | 42 aa | ALYLLRRDQRLPPDAHKPPGGGSFRTPIQEEQADAHSTL |
| | | | AKI (SEQ ID NO: 39) |
| 2B4 | NM_016382 | 120 aa | AWRRKRKEKQSETSPKEFLTIYEDVKDLKTRRNHEQEQT |
| | | | FPGGGSTIYSMIQSQSSAPTSQEPAYTLYSLIQPSRKSG |
| | | | SRKRNHSPSFNSTIYEVIGKSQPKAQNPARLSRKELENF |
| | | | DVYS (SEQ ID NO: 40) |

In some examples, the CD3ζ signaling domain comprises an amino acid sequence that is at least 90%, at least 95%, at least 98% identical to SEQ ID NO: 35. In such instances, the CD3ζ signaling domain has 1, 2, 3, 4, or 5 amino acid changes (preferably conservative substitutions) compared to SEQ ID NO: 35. In other examples, the CD3ζ signaling domain is SEQ ID NO: 35.

In various embodiments: the co-stimulatory domain is selected from the group consisting of: a co-stimulatory domain depicted in Table 3 or a variant thereof having 1-5 (e.g., 1 or 2) amino acid modifications, a CD28 co-stimulatory domain or a variant thereof having 1-5 (e.g., 1 or 2) amino acid modifications, a 4-1 BB co-stimulatory domain or a variant thereof having 1-5 (e.g., 1 or 2) amino acid modifications and an OX40 co-stimulatory domain or a variant thereof having 1-5 (e.g., 1 or 2) amino acid modifications. In certain embodiments, a 4-1 BB co-stimulatory domain or a variant thereof having 1-5 (e.g., 1 or 2) amino acid modifications is present in the CAR polypeptides described herein.

In some embodiments, there are two co-stimulatory domains, for example, a CD28 co-stimulatory domain or a variant thereof having 1-5 (e.g., 1 or 2) amino acid modifications (e.g., substitutions) and a 4-1 BB co-stimulatory domain or a variant thereof having 1-5 (e.g., 1 or 2) amino acid modifications (e.g., substitutions). In various embodiments the 1-5 (e.g., 1 or 2) amino acid modification are substitutions. In various embodiments, the co-stimulatory domain is amino terminal to the CD3ζ signaling domain and a short linker consisting of 2-10, e.g., 3 amino acids (e.g., GGG) is can be positioned between the co-stimulatory domain and the CD3ζ signaling domain.

In some cases, the CAR can be produced using a vector in which the CAR open reading frame is followed by a T2A ribosome skip sequence and a truncated EGFR (EGFRt), which lacks the cytoplasmic signaling tail, or a truncated CD19R (also called CD19t). In this arrangement, co-expression of EGFRt or CD19t provides an inert, non-immunogenic surface marker that allows for accurate measurement of gene modified cells, and enables positive selection of gene-modified cells, as well as efficient cell tracking of the therapeutic T cells in vivo following adoptive transfer. Efficiently controlling proliferation to avoid cytokine storm and off-target toxicity is an important hurdle for the success of T cell immunotherapy. The EGFRt or the CD19t incorporated in the CAR lentiviral vector can act as suicide gene to ablate the CAR+ T cells in cases of treatment-related toxicity.

The CD3ζ signaling domain can be followed by a ribosomal skip sequence (e.g., LEGGGEGRGSLLTCGDVEENPGPR; SEQ ID NO: 45) and a truncated EGFR having a sequence that is at least 90%, at least 95%, at least 98% identical to or identical to: LVTSLLLCELPHPAFLLIPRKVCNGIGIGEFKDSLSINATNIKHFKNCTSISGDLHILPVAFRGD SFTHTPPLDPQELDILKTVKEITGFLLIQAWPENRTDLHAFENLEIIRGRTKQHGQFSLAVVSL NITSLGLRSLKEISDGDVIISGNKNLCYANTINWKKLFGTSGQKTKIISNRGENSCKATGQVC HALCSPEGCWGPEPRDCVSCRNVSRGRECVDKCNLLEGEPREFVENSEClQCHPECLPQ AMNITCTGRGPDNCIQCAHYIDGPHCVKTCPAGVMGENNTLVWKYADAGHVCHLCHPNCT YGCTGPGLEGCPTNGPKIPSIATGMVGALLLLLVVALGIGLFM (SEQ ID NO: 46). In some cases, the truncated EGFR has 1, 2, 3, 4 of 5 amino acid changes (preferably conservative) compared to SEQ ID NO: 46.

Alternatively the CD3ζ signaling domain can be followed by a ribosomal skip sequence (e.g., LEGGGEGRGSLLTCGDVEENPGPR; SEQ ID NO: 45) and a truncated CD19R (also called CD19t) having a sequence that is at least 90%, at least 95%, at least 98% identical to or identical to:

(SEQ ID NO: 47)
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCVPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKR.

The CAR or polypeptide described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably a T lymphocyte, and most preferably an autologous T lymphocyte.

Various T cell subsets isolated from the patient can be transduced with a vector for CAR or polypeptide expression. Central memory T cells are one useful T cell subset. Central memory T cell can be isolated from peripheral blood mononuclear cells (PBMC) by selecting for CD45RO+/CD62L+ cells, using, for example, the CliniMACS® device to immunomagnetically select cells expressing the desired receptors. The cells enriched for central memory T cells can be activated with anti-CD3/CD28, transduced with, for example, a lentiviral vector that directs the expression of the CAR or as well as a non-immunogenic surface marker for in vivo detection, ablation, and potential ex vivo selection. The activated/genetically modified central memory T cells can be expanded in vitro with IL-2/IL-15 and then cryopreserved. Additional methods of preparing CAR T cells can be found in PCT/US2016/043392.

Methods for preparing useful T cell populations are described in, for example, WO 2017/015490 and WO 2018/102761. In some cases, it may be useful to use natural killer (NK) cells, e.g., allogenic NK cells derived from peripheral blood or cord blood. In other cases, NK cells can be derived from human embryonic stem cells (hESCs) or induced pluripotent stem cells (iPSCs).

In some embodiments, described herein is a composition comprising the iPSC-derived CAR T cells or CAR NK cells. In some embodiments, a composition comprising iPSC-derived CAR T cells or CAR NK cells has enhanced therapeutic properties. In some embodiments, the iPSC-derived CAR T cells or CAR NK cells demonstrate enhanced functional activity including potent cytokine production, cytotoxicity and cytostatic inhibition of tumor growth, e.g., as activity that reduces the amount of tumor load.

The CAR can be transiently expressed in a T cell population by an mRNA encoding the CAR. The mRNA can be introduced into the T cells by electroporation (Wiesinger et al. 2019 Cancers (Basel) 11:1198).

In some embodiments, a composition comprising the CAR T cells comprise one or more of helper T cells, cytotoxic T cells, memory T cells, naïve T cells, regulatory T cells, natural killer T cells, or combinations thereof.

Suitable TAG-72 targeted CAR are described in, e.g., WO 2020/028721. Suitable HER2 targeted CAR are described in, e.g., WO 2017/079694; suitable PSCA targeted CAR are described in WO 2017/062628.

In some cases, the TAG-72 CAR comprises the amino acid sequence:

(SEQ ID NO: 61)
QVQLVQSGAEVVKPGASVKISCKASGYTFTDHAIHWVKQNPGQRLEWIGY
FSPGNDDEKYSQKFQGKATLTADTSASTAYVELSSLRSEDTAVYFCTRSL
NMAYWGQGTLVTVSSGSTSGGGSGGGSGGGGSSDIVMSQSPDSLAVSLGE
RVTLNCKSSQSVLYSSNSKNYLAWYQQKPGQSPKLLIYWASTRESGVPDR
FSGSGSGTDFTLTISSVQAEDVAVYYCQQYYSYPLSFGAGTKLELKESKY
GPPCPPCPGGGSSGGGSGGQPREPQVYTLPPSQEEMTKNQVSLTCLVKGF
YPSDIAVEWESNGQPENNYKTTPPVLDSDGSFFLYSRLTVDKSRWQEGNV
FSCSVMHEALHNHYTQKSLSLSLGKMFWVLVVVGGVLACYSLLVTVAFII
FWVKRGRKKLLYIFKQPFMRPVQTTQEEDGCSCRFPEEEEGGCELGGGRV
KFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYD
ALHMQALPPR

Thus, it includes, from amino to carboxy terminus, an scFv (underlined) and spacer (not underlined) a CD28 transmembrane domain (MFWVLVVVGGVLACYSLLVTVAFIIFWV, SEQ ID NO:17; underlined), a 4-1 BB co-stimulatory domain (KRGRKKLLYIFKQPFMRPVQTTQEEDGCSCRFPEEEEGGCEL, SEQ ID NO:38; not underlined), a GGG linker (GGG; underlined) and a CD3zeta domain (RVKFSRSADAPAYQQGQNQLYNELNLGRREEYDVLDKRRGRDPEMGGKPRRKNPQEGL YNELQKDKMAEAYSEIGMKGERRRGKGHDGLYQGLSTATKDTYDALHMQALPPR, SEQ ID NO:35; not underlined). The scFv includes a VH chain comprising the amino acid sequence: QVQLVQSGAEVVKPGASVKISCKASGYTFTDHAIHWVKQNPGQRLEWIGYFSPGNDDFKY SQKFQGKATLTADTSASTAYVELSSLRSEDTAVYFCTRSLNMAYWGQGTLVTVSS (SEQ ID NO: 63). This VH chain includes: VH CDR1 (DHAIH, SEQ ID NO:64); VH CDR2 (YFSPGNDDFKYSQKFQG, SEQ ID NO:65); and VH CDR3 (SLNMAY, SEQ ID NO:66). The scFv includes a VL chain comprising the amino acid sequence:

(SEQ ID NO: 67)
DIVMSQSPDSLAVSLGERVTLNCKSSQSVLYSSNSKNYLAWYQQKPGQSP
KLLIYWASTRESGVPDRFSGSGSGTDFTLTISSVQAEDVAVYYCQQYYSY
PLSFGAGTKLELK.
This VL chain includes VL CDR1 (KSSQSVLYSSNSKNYLA,
SEQ ID NO: 68);
VL
CDR2 (WASTRES, SEQ ID NO: 69);
and
VL
CDR3 (QQYYSYPLS, SEQ ID NO: 70).

The spacer includes: an IgG4 hinge with a S to P mutation (ESKYGPPCPECP, SEQ ID NO:25; mutation in bold, underline), a linker (GGGSSGGGSG, SEQ ID NO:24), and an IgG4 CH3 domain

(GQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQK
SLSLSLGK, SEQ ID NO: 34).

In some cases, the mCD28 transmembrane domain can be replaced by the following CD28 transmembrane domain: FWVLVVVGGVLACYSLLVTVAFIIFWV (SEQ ID NO:16).

In some cases, the TAG72 includes a signal sequence at the amino terminus, e.g. a GMCSF signal sequence (MLLLVTSLLLCELPHPAFLLIP; SEQ ID NO:11). Thus, the TAG72 CAR with a signal sequence can be:

(SEQ ID NO: 62)
MLLLVTSLLLCELPHPAFLLIPQVQLVQSGAEVVKPGASVKISCKASGYT
FTDHAIHWVKQNPGQRLEWIGYFSPGNDDEKYSQKFQGKATLTADTSAST
AYVELSSLRSEDTAVYFCTRSLNMAYWGQGTLVTVSSGSTSGGGSGGGSG
GGGSSDIVMSQSPDSLAVSLGERVTLNCKSSQSVLYSSNSKNYLAWYQQK
PGQSPKLLIYWASTRESGVPDRESGSGSGTDETLTISSVQAEDVAVYYCQ
QYYSYPLSFGAGTKLELKESKYGPPCPPCPGGGSSGGGSGGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGKMFW
VLVVVGGVLACYSLLVTVAFIIFWVKRGRKKLLYIFKQPFMRPVQTTQEE
DGCSCRFPEEEEGGCELGGGRVKFSRSADAPAYQQGQNQLYNELNLGRRE
EYDVLDKRRGRDPEMGGKPRRKNPQEGLYNELQKDKMAEAYSEIGMKGER
RRGKGHDGLYQGLSTATKDTYDALHMQALPPR

In some cases, the TAG72 CAR can be followed by a T2A skip sequence and a truncated CD19 have can serve as a marker:

(SEQ ID NO: 71)
LEGGGEGRGSLLTCGDVEENPGPTRMPPPRLLFFLLFLTPMEVRPEEPLV
VKVEEGDNAVLQCLKGTSDGPTQQLTWSRESPLKPFLKLSLGLPGLGIHM
RPLAIWLFIFNVSQQMGGFYLCQPGPPSEKAWQPGWTVNVEGSGELFRWN
VSDLGGLGCGLKNRSSEGPSSPSGKLMSPKLYVWAKDRPEIWEGEPPCVP
PRDSLNQSLSQDLTMAPGSTLWLSCGVPPDSVSRGPLSWTHVHPKGPKSL
LSLELKDDRPARDMWVMETGLLLPRATAQDAGKYYCHRGNLTMSFHLEIT
ARPVLWHWLLRTGGWKVSAVTLAYLIFCLCSLVGILHLQRALVLRRKR

### HER2 CAR

In some cases the HER2 CAR comprises the amino acid sequence:

(SEQ ID NO: 72)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKGSTSGGGSGGGSGGGGSSEVQLVESGGGLVQPGGSLRLSCAAS
GFNIKDTYIHWVRQAPGKGLEWVARIYPTNGYTRYADSVKGRFTISADTS
KNTAYLQMNSLRAEDTAVYYCSRWGGDGFYAMDYWGQGTLVTVSSESKYG
PPCPPCPAPEFEGGPSVFLFPPKPKDTLMISRTPEVTCVVVDVSQEDPEV
QFNWYVDGVEVHNAKTKPREEQFQSTYRVVSVLTVLHQDWLNGKEYKCKV
SNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVF
SCSVMHEALHNHYTQKSLSLSLGKIYIWAPLAGTCGVLLLSLVITKRGRK
KLLYIFKQPFMRPVQTTQEEDGCSCRFPEEEEGGCELGGGRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PR

Thus, it includes, from amino to carboxy terminus, an scFv (underlined) and spacer (not underlined) a CD8 transmembrane domain (underlined), a 4-1 BB co-stimulatory domain (not underlined), a GGG linker (underlined) and a CD3zeta domain (not underlined). The spacer in this HER2 CAR is IgG4(S228P, L235E,N297Q).

### PSCA CAR

### Suitable PSCA CAR Include:

PSCAscFv-IgG4(HL-CH3)-CD4tm-41BB-Zeta
(SEQ ID NO: 73)
DIQLTQSPSTLSASVGDRVTITCSASSSVRFIHWYQQKPGKAPKRLIYDT
SKLASGVPSRFSGSGSGTDFTLTISSLOPEDFATYYCQQWGSSPFTFGQG
TKVEIKGSTSGGGSGGGSGGGGSSEVQLVEYGGGLVQPGGSLRLSCAASG
FNIKDYYIHWVRQAPGKGLEWVAWIDPENGDTEFVPKFQGRATMSADTSK
NTAYLQMNSLRAEDTAVYYCKTGGFWGQGTLVTVSSESKYGPPCPPCPGG
GSSGGGSGGQPREPQVYTLPPSQEEMTKNQVSLTCLVKGFYPSDIAVEWE
SNGQPENNYKTTPPVLDSDGSFFLYSRLTVDKSRWQEGNVFSCSVMHEAL
HNHYTQKSLSLSLGKMALIVLGGVAGLLLFIGLGIFFKRGRKKLLYIFKQ
PFMRPVQTTQEEDGCSCRFPEEEEGGCELGGGRVKFSRSADAPAYQQGQN
QLYNELNLGRREEYDVLDKRRGRDPEMGGKPRRKNPQEGLYNELQKDKMA
EAYSEIGMKGERRRGKGHDGLYQGLSTATKDTYDALHMQALPPR
PSCAscFv-Linker-CD4tm-41BB-Zeta
(SEQ ID NO: 74)
DIQLTQSPSTLSASVGDRVTITCSASSSVRFIHWYQQKPGKAPKRLIYDT
SKLASGVPSRFSGSGSGTDFTLTISSLEPEDFATYYCQQWGSSPFTFGQG
TKVEIKGSTSGGGSGGGSGGGGSSEVELVEYGGGLVQPGGSLRLSCAASG
FNIKDYYIHWVRQAPGKGLEWVAWIDPENGDTEFVPKFQGRATMSADTSK
NTAYLQMNSLRAEDTAVYYCKTGGFWGQGTLVTVSSGGGSSGGGSGMALI
VLGGVAGLLLFIGLGIFFKRGRKKLLYIFKQPFMRPVQTTQEEDGCSCRF
PEEEEGGCELGGGRVKFSRSADAPAYQQGQNQLYNELNLGRREEYDVLDK
RRGRDPEMGGKPRRKNPQEGLYNELQKDKMAEAYSEIGMKGERRRGKGHD
GLYQGLSTATKDTYDALHMQALPPR
PSCAscFv-IgG4(S228P, L235E, N297Q)-
CD4tm-41BB-Zeta
(SEQ ID NO: 75)
DIQLTQSPSTLSASVGDRVTITCSASSSVRFIHWYQQKPGKAPKRLIYDT
SKLASGVPSRFSGSGSGTDFTLTISSLOPEDFATYYCQQWGSSPFTFGQG
TKVEIKGSTSGGGSGGGSGGGGSSEVQLVEYGGGLVQPGGSLRLSCAASG
FNIKDYYIHWVRQAPGKGLEWVAWIDPENGDTEFVPKFQGRATMSADTSK
NTAYLQMNSLRAEDTAVYYCKTGGFWGQGTLVTVSSESKYGPPCPPCPAP
EFEGGPSVFLFPPKPKDTLMISRTPEVTCVVVDVSQEDPEVQFNWYVDGV
EVHNAKTKPREEQFQSTYRVVSVLTVLHQDWLNGKEYKCKVSNKGLPSSI
EKTISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHEAL
HNHYTQKSLSLSLGKMALIVLGGVAGLLLFIGLGIFFKRGRKKLLYIFKQ
PFMRPVQTTQEEDGCSCRFPEEEEGGCELGGGRVKFSRSADAPAYQQGQN
QLYNELNLGRREEYDVLDKRRGRDPEMGGKPRRKNPQEGLYNELQKDKMA
EAYSEIGMKGERRRGKGHDGLYQGLSTATKDTYDALHMQALPPR

### Treatment of Cancer

Aspects of the present disclosure provide methods for treating a subject having a cancer by administering immune cells, e.g., T cells that express mb(28)lL-12 and a chimeric antigen receptor.

### (a) Subjects

The subject to be treated by the methods described can be a human patient having solid tumor, e.g., gastrointestinal cancer, breast cancer, lung cancer, bladder cancer, thyroid cancer, endometrial cancer, esophageal cancer, and ovarian cancer. Non-limiting examples of gastrointestinal cancers include colon cancer, gastric cancer, rectal cancer, colorectal cancer, pancreatic cancer, and combinations thereof.

A subject at risk of having cancer might show one or more symptoms of a cancer, e.g., unexplained weight loss, fatigue, pain, persistent cough, lumps under the skin, or unusual bleeding. A subject at risk of having cancer might have one or more risk factors of a cancer, e.g., family history of cancer, age, tobacco use, obesity, or exposure to sun or carcinogens. A subject who needs the treatment described herein can be identified by routine medical examination, e.g., laboratory tests, biopsy, magnetic resonance imaging (MRI), or ultrasound exams.

### (b) Treatment Regimens

Aspects of the present disclosure provide methods of treating a solid tumor comprising administering a lymphodepletion treatment (e.g., cyclophosphamide) in combination with immune cells expressing mb(28)IL-12 and an appropriate chimeric antigen receptor. In some cases, the treatment also includes an inhibitor of PD-L1 activity such as an anti-PD-L1 antibody of an anti-PD-1 antibody or some other therapeutic that reduces immunosuppression. The two components can be administered the same day or on different days.

Any subject suitable for the treatment methods described herein can receive a lymphodepleting therapy to reduce or deplete the endogenous lymphocytes of the subject. Lymphodepletion refers to the destruction of endogenous lymphocytes and/or T cells, which is commonly used prior to immunotransplantation and immunotherapy. Lymphodepletion can be achieved by administering a lymphodepleting agent and/or irradiation (e.g., stereotactic radiation). A lymphodepleting agent can be any molecule capable of reducing, depleting, or eliminating endogenous lymphocytes and/or T cells when administered to a subject. In some examples, the lymphodepleting agents are cytotoxic agents that specifically kill lymphocytes. Non-limiting examples of lymphodepleting agents include cyclophosphamide, fludarabine, gemcitabine, methotrexate, doxorubicin, and etopside phosphate. In some cases the lymphodepletion treatment is non-myeloablative.

Methods described herein can include a conditioning regimen comprising a single lymphodepleting agent (e.g., cyclophosphamide) or multiple lymphodepleting agents (e.g., cyclophosphamide and fludarabine). The subject to be treated by the methods described herein can receive one or more doses of the one or more lymphodepleting agents for a period suitable for reducing or depleting the endogenous lymphocytes of the subject (e.g., 1-5 days).

The subject can then be administered any of CAR immune cells described herein after administration of the lymphodepleting therapy as described herein. For example, the one or more lymphodepleting agents can be administered to the subject 1-5 days (e.g., 1, 2, 3, 4, or 5 days) prior to administering the CAR immune cells, e.g., CAR T cells.

Methods described herein can include redosing the subject with CAR immune cells. In some examples, the subject is administered a lymphodepleting treatment prior to redosing of the anti-CAR immune cells. Each dose of the CAR immune cells can be the same or the doses can be ascending or descending.

Methods described herein can be used in combination with another anti-cancer therapy (e.g., chemotherapy) or with another therapeutic agent that reduces side effects of the therapy described herein.

### (c) Administration

An effective amount of a therapy (e.g., lymphodepleting agent, T cells expressing a CAR) can be administered to a subject (e.g., a human) in need of the treatment via any suitable route (e.g., administered locally or systemically to a subject). Suitable modes of administration include injection, infusion, instillation, or ingestion. Injection includes, without limitation, intravenous, intramuscular, intra-arterial, intrathecal, intraventricular, intradermal, intraperitoneal, and subcutaneous injection and infusion.

An effective amount refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of treatment, the nature of concurrent therapy, if any, the specific route of administration and like factors.

The materials, methods, and examples are illustrative only and not intended to be limiting. All publications, patent applications, patents, sequences, database entries, and other references mentioned herein are incorporated by reference in their entirety for any and all purposes.

Other features and advantages of the described compositions and methods will be apparent from the following detailed description and figures, and from the claims.

### EXAMPLES

In order that the invention described may be more fully understood, the following examples are set forth. The materials and method used in the Examples below are detailed following the Examples. The examples described in this application are offered to illustrate the methods and compositions provided herein and are not to be construed in any way as limiting their scope.

### Example 1: CD28 Transmembrane in TAG72-CARs Containing a 4-1 BB Costimulatory Domain Enhances Anti-Tumor Activity In Vitro

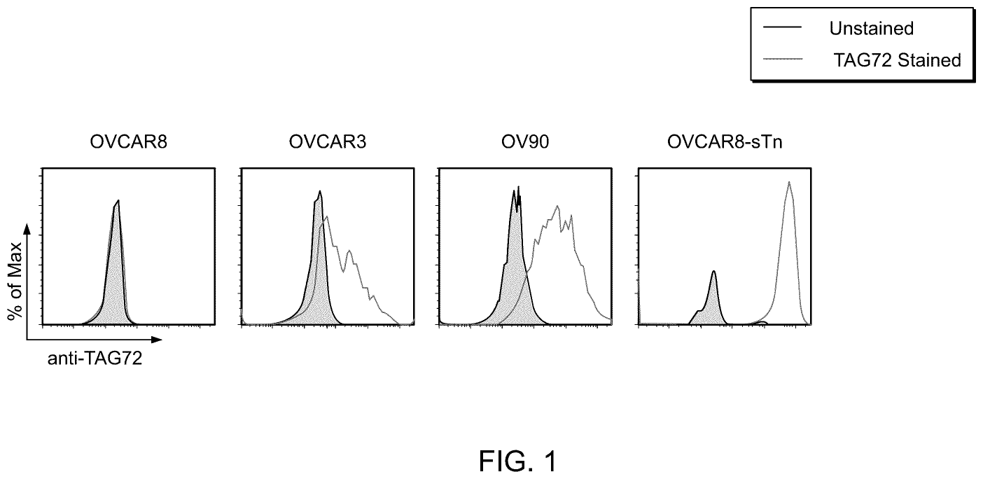

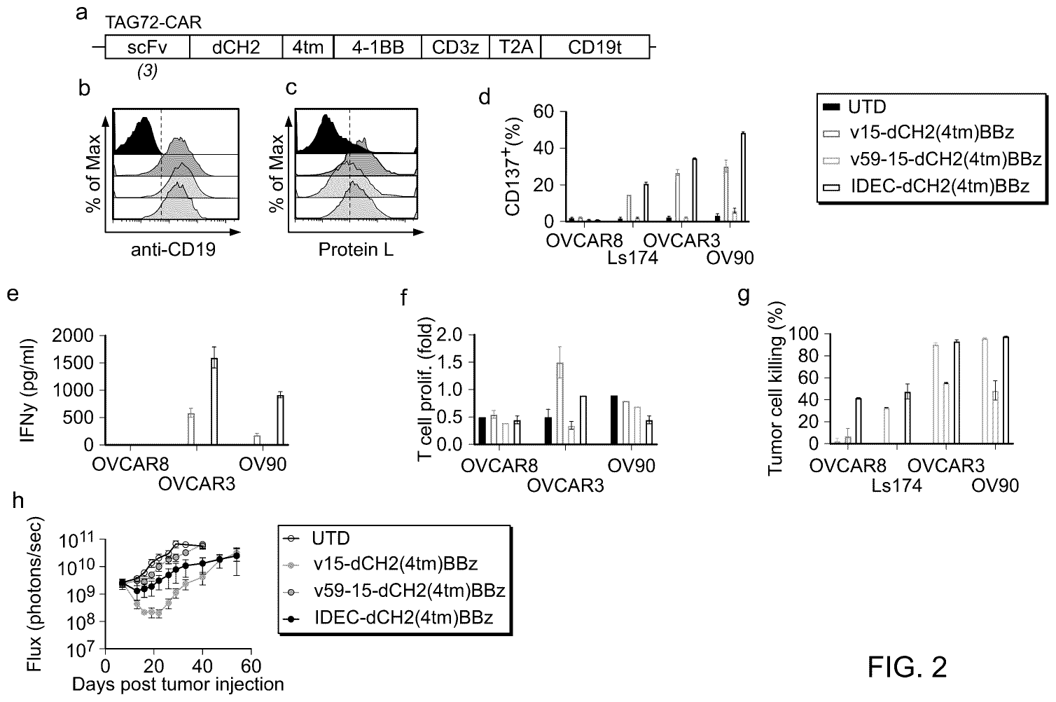

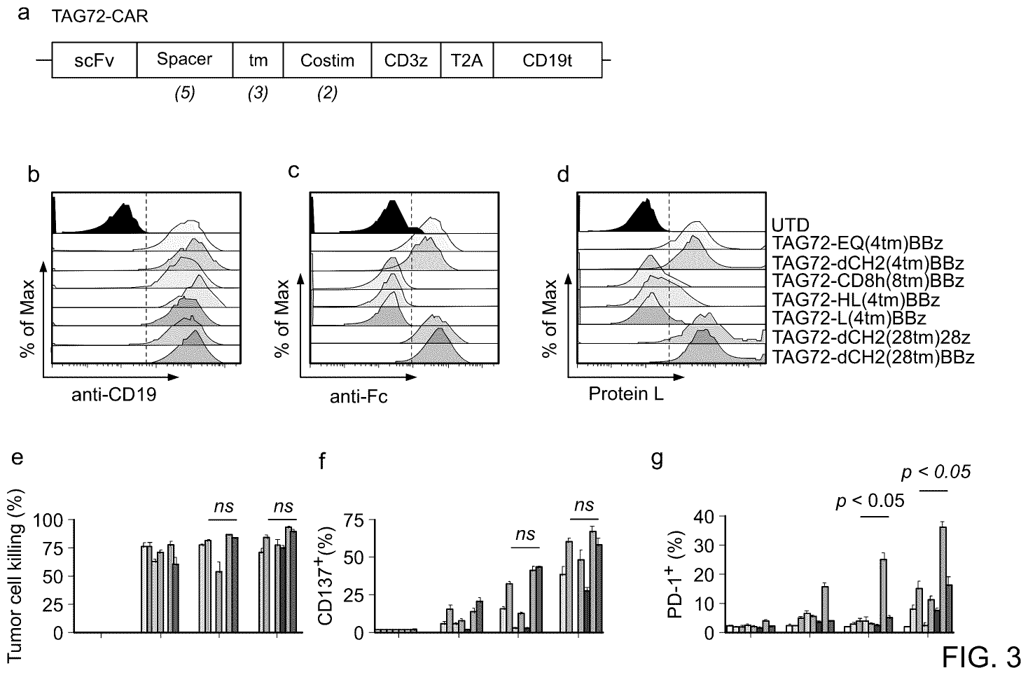

We previously generated and pre-clinically evaluated second-generation TAG72-specific CAR T cells containing a 4-1 BB intracellular costimulatory domain, which demonstrated potent anti-tumor activity using human xenograft peritoneal ovarian tumor models³¹. Based on our preclinical studies showing a lack of curative anti-tumor activity, and early phase 1 data using first-generation TAG72-CAR T cells³³ demonstrating anti-idiotype antibody production in patients likely contributing to a lack of durable therapeutic responses, we decided to redesign the CAR molecule for optimal functionality. First, we re-assessed the antigen-binding single chain variable fragment (scFv) domain of our TAG72-CAR construct in attempts to minimize the potential for anti-CAR immunogenicity and improve T cell persistence. We utilized two additional scFvs (v15, and v59-15: a fusion between v15 and v59) based on the original humanized CC49 scFv (IDEC) that through affinity maturation showed reduced potential for anti-idiotype immunogenicity^{34,35}. Two of three scFvs exhibited similar high binding affinities toward TAG72 antigen (IDEC, K_{D}=33±20 nM; v15, K_{D}=35±10 nM; v59-15, not determined). For all related in vitro and in vivo studies, we use human ovarian cancer cell lines that are TAG72-negative (OVCAR8) or are varying in cell surface expression levels of TAG72 (OVCAR3, OV90, and OVCAR8-sTn) (FIG. 1). We incorporated these scFvs into the same CAR backbone we originally published³¹, and evaluated their anti-tumor activity in vitro and in vivo (FIG. 2a-2h). TAG72-CAR T cells with the v15 scFv was the most optimal in terms of anti-tumor activity as compared with IDEC and v59-15.

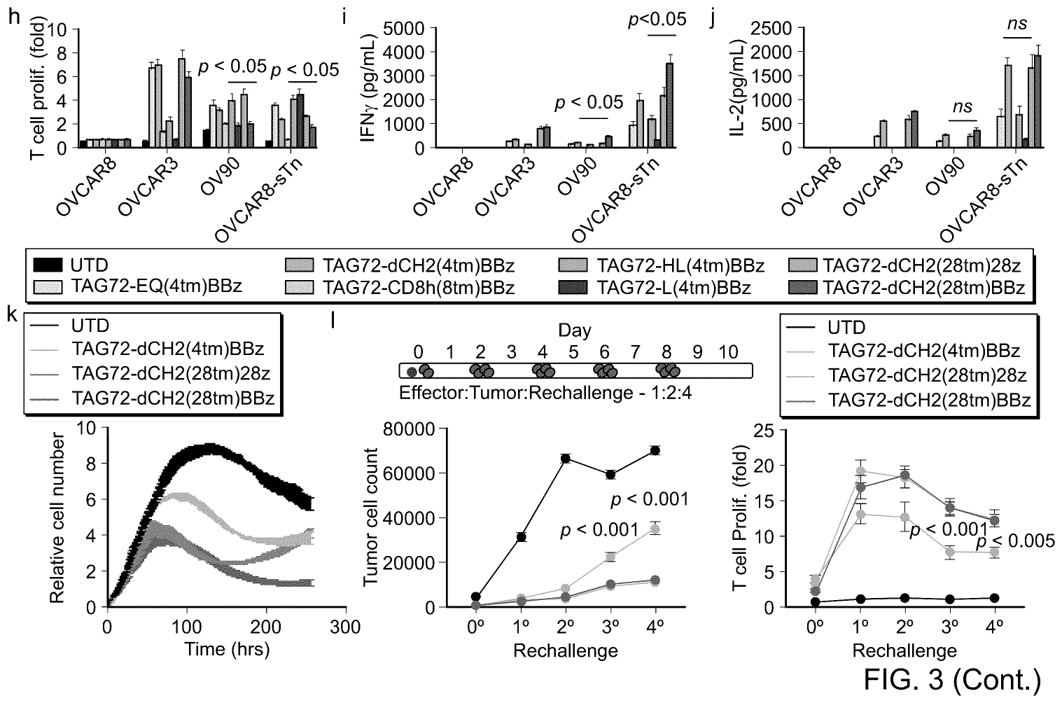

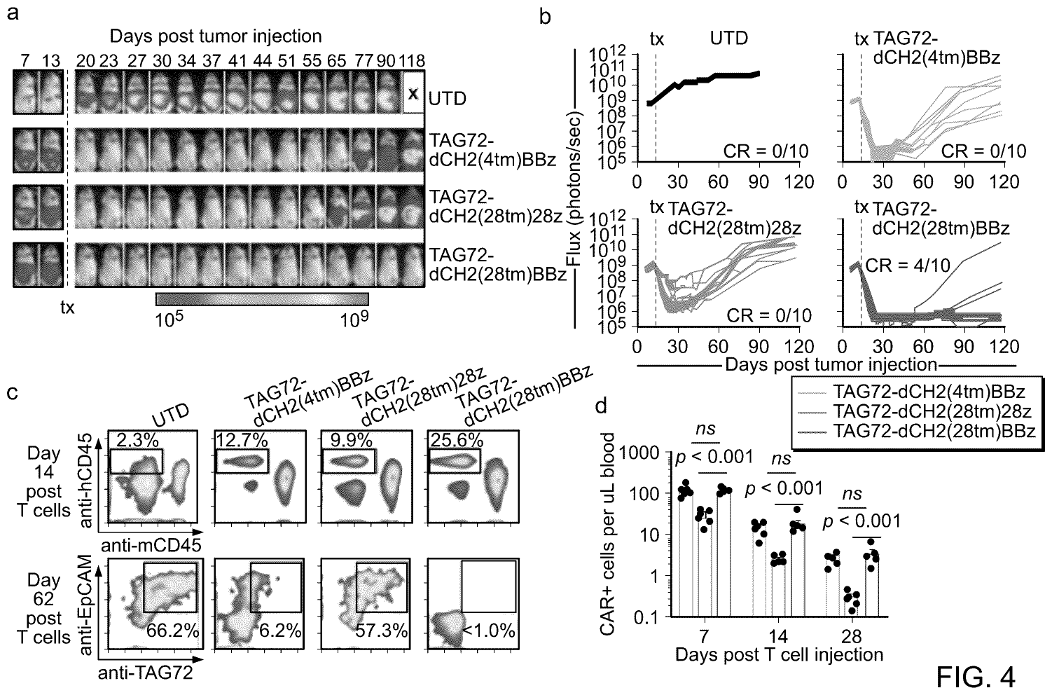

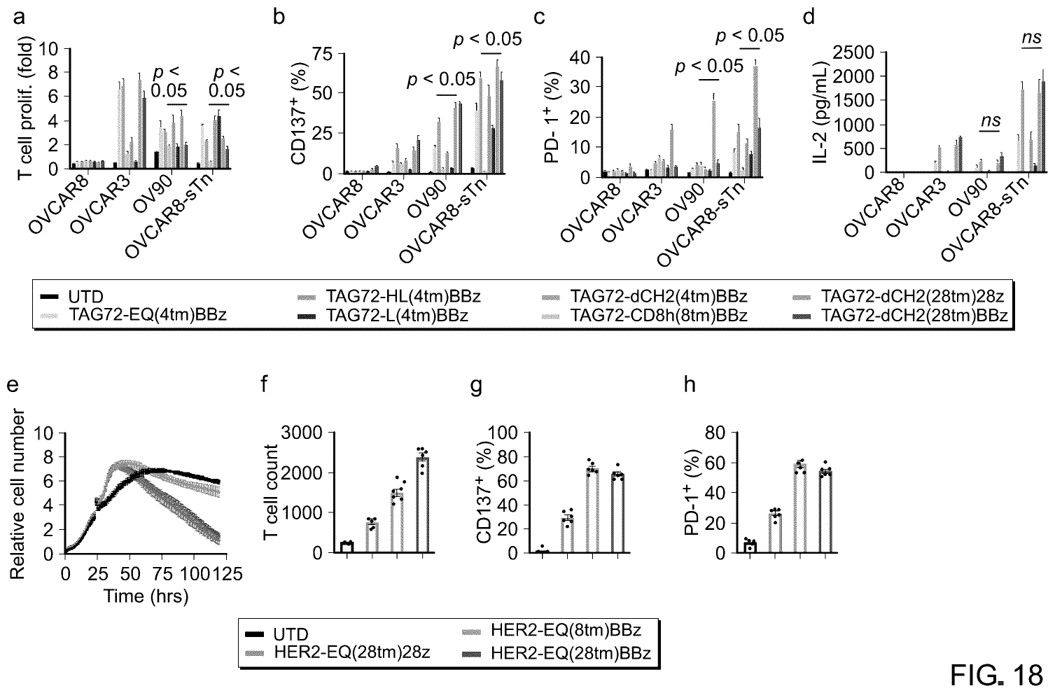

Next, we generated seven v15 scFv-based TAG72-CAR constructs with varying extracellular spacer domains and lengths (termed EQ, dCH2, CD8h, HL, and L), transmembrane domains (CD4tm, CD8tm, CD28tm), and intracellular costimulatory domains (CD28, 4-1 BB) (FIG. 3a). While all seven CAR molecules comparably expressed CD19t marker, we saw higher cell surface CAR expression with Fc-derived spacers (EQ, dCH2), as measured by Protein L staining of the scFv, which closely matched the staining pattern using anti-Fc antibodies to detect the extracellular spacer domain (FIG. 3b-3d and FIGS. 18a, 18b, and 18d). The three TAG72-CAR leads (dCH2(28tm)28z, dCH2(4tm)BBz, and dCH2(28tm)BBz) showed highest T cell activation and cytokine production. Additionally, we showed the greatest PD-1 exhaustive phenotype in CD28 costimulatory domain-containing CAR T cells (FIG. 18c), in line with previous reports using other CARs. Interestingly, we showed the lowest PD-1 and highest IFNγ with TAG72-CAR T cells that contained the CD28tm and 4-1 BB costimulatory domain. Some of these data were confirmed using HER2-CAR T cells, showing the greatest tumor cell killing, highest CD137 activation, and enhanced antigen-dependent T cell proliferation when CD28tm was coupled with the 4-1 BB costimulatory domain (FIG. 18e-18h).

We next evaluated cytotoxicity function of these TAG72-CAR T cells using in vitro coculture killing assays against cancer cell lines with varying TAG72 expression. In general, we found that TAG72-CAR T cells containing the dCH2 spacer domain showed superior functionality with the greatest tumor cell killing, highest CD137 activation, reduced PD-1 expression, enhanced antigen-dependent T cell proliferation, and robust IFNγ and IL-2 cytokine production (FIG. 3e-i). The three TAG72-CAR leads (dCH2(4tm)BBz, dCH2(28tm)BBz, and dCH2(28tm)28z) showed highest antigen density-dependent T cell activation and cytokine production. Additionally, we showed the greatest PD-1 exhaustive phenotype in CD28 costimulatory domain-containing CAR T cells, similar to prior reports with other CARs^{16,36-38} Unexpectedly, while TAG72-CAR variants with CD8h and HL spacers showed an apparent lack of CAR cell surface expression, they showed varying tumor cell killing potential with some capacity, albeit suboptimal, to induce antigen-dependent T cell proliferation. Finally, we showed the lowest PD-1 expression and highest IFNγ production with our TAG72-CAR T cells that contained the CD28tm and 4-1 BB costimulatory domain.

From these studies, we proceeded to further “stress-test” the three TAG72-CAR T cell lead candidates. We first evaluated cytotoxicity potential of CAR T cells over an extended 10-day coculture assay with OV90 tumor cells. Using xCELLigence as a readout, TAG72-CAR T cells containing the CD28tm and 4-1 BB costimulatory domain displayed the greatest anti-tumor activity (FIG. 3k). We then performed recursive tumor cell killing assays by rechallenging TAG72-CAR T cells with OV90 tumor cells. We observed two intriguing patterns; both 4-1 BB costimulatory domain-containing CAR showed superior antigen-dependent T cell expansion profiles, whereas the CD28 transmembrane domain-containing CAR T cells achieved better control of tumors over the rechallenge time points (FIG. 3l). Collectively, our in vitro studies have identified three TAG72-CAR T cell lead candidates with potent but varying anti-tumor functional profiles, which we selected for further assessment of their in vivo preclinical therapeutic activity.

### Example 2: CD28 Transmembrane Domain in 4-1 BB-Based TAG72-CAR T Cells Induces Durable Therapeutic Responses In Vivo

Anti-tumor efficacy of these three TAG72-CAR leads was evaluated using previously established human peritoneal ovarian tumor xenograft models³¹. At day 14 following intraperitoneal (i.p.) OVCAR3 tumor injection, mice were treated with untransduced (UTD), TAG72-dCH2(4tm)BBz, TAG72-dCH2(28tm)28z, or TAG72-dCH2(28tm)BBz CAR T cells (5.0×10⁶) by regional i.p. delivery. Dramatic anti-tumor responses were shown with all three CAR T cells, which was sustained for up to 6 weeks in treated mice (FIG. 4a). However, we observed tumor recurrences after 6-8 weeks post-treatment in mice treated with TAG72-dCH2(4tm)BBz or TAG72-dCH2(28tm)28z CAR T cells, whereas TAG72-dCH2(28tm)BBz durably controlled tumors resulting in 4 out of 10 mice achieving complete therapeutic responses (FIG. 4b).

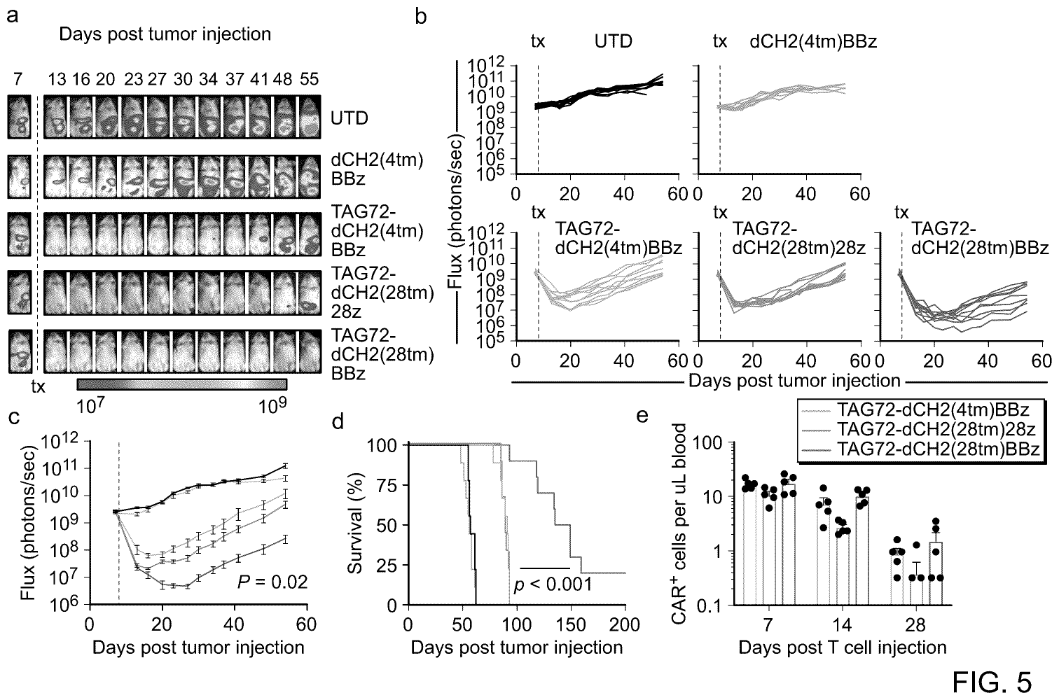

To uncover the potential differences observed between the three TAG72-CAR T cell leads, we quantified CAR T cells in the peritoneal ascites and peripheral blood of mice following therapy. CAR T cells were seen in peritoneal ascites in treated mice, with TAG72-dCH2(28tm)BBz CAR T cells showing the greatest proportion of cells collected from the ascites 14 days after CAR T cell injection, compared with the other two CAR T cells (FIG. 4c). We additionally collected peritoneal ascites at 62 days post-treatment, at the time of tumor recurrences, and showed elimination of TAG72+ tumor cells in TAG72-dCH2(28tm)BBz CAR T cell-treated mice, but the presence of TAG72+ tumor cells with the other two CAR T cells (FIG. 4c). We observed similar number of 4-1 BB costimulatory domain-containing CAR T cells in the blood of mice at all three timepoints, higher than CD28 costimulatory domain-containing CAR T cells (FIG. 4d). These data largely matched the T cell proliferation pattern in our in vitro functional assays. We further observed similar anti-tumor kinetics of the three CAR T cell lead candidates using a second, more aggressive, human OV90 peritoneal ovarian tumor xenograft model (FIG. 5).

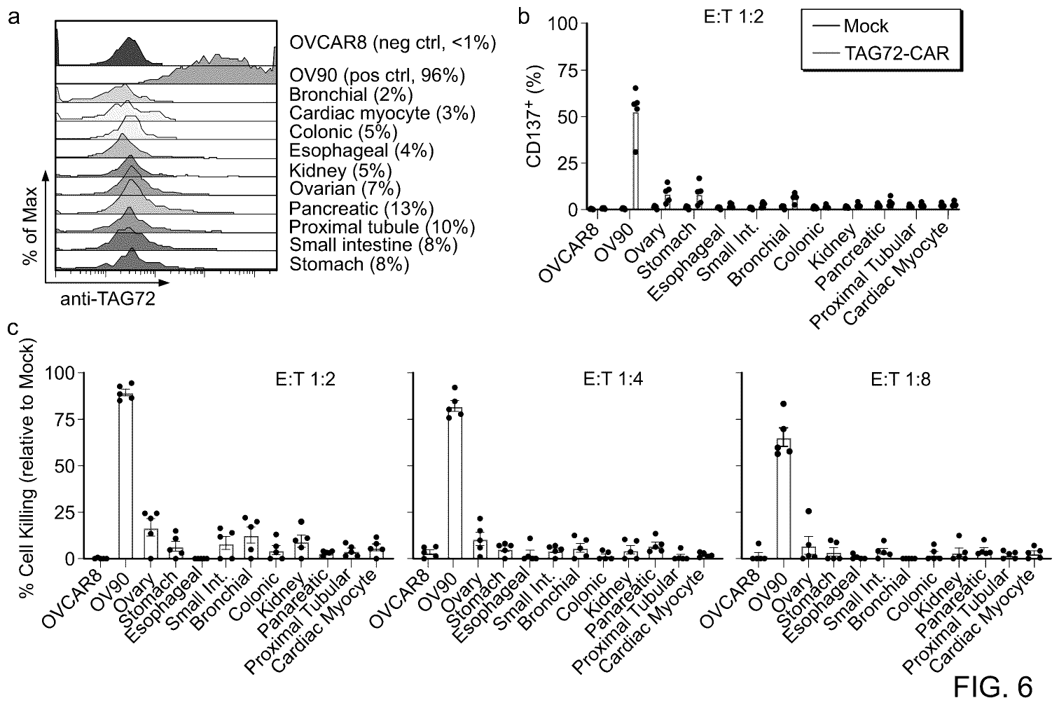

To better address potential safety concerns of this optimized TAG72-CAR containing a new anti-TAG72 scFv and CAR backbone, we evaluated the on- and off-target normal cell killing potential of TAG72-dCH2(28tm)BBz CAR T cells. Little to no TAG72 expression was observed across 10 primary human normal cells evaluated by flow cytometry, with minimal CD137+ T cell activation and cell killing by TAG72-CAR T cells, a finding that was consistent across five independent human CAR T cell donors (FIG. 6). In sum, the TAG72-dCH2(28tm)BBz CAR construct showed the most optimal anti-tumor functionality in vitro with safe, potent, and durable anti-tumor efficacy in vivo.

### Example 3: IL-12/IFNγ Signaling Regulates the Anti-Tumor Activity of CAR T Cells

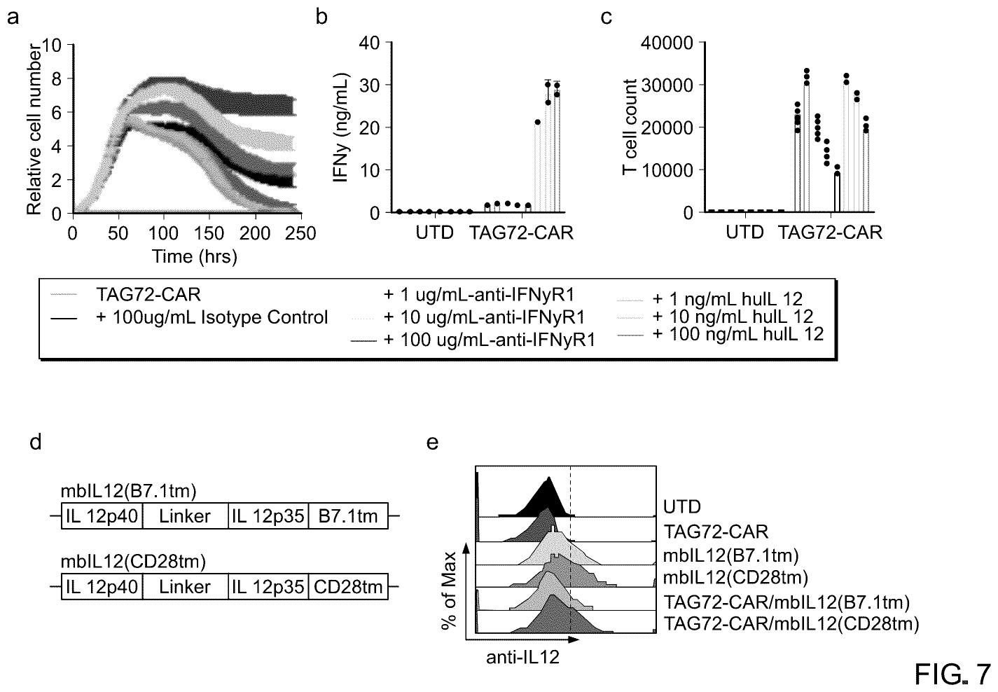

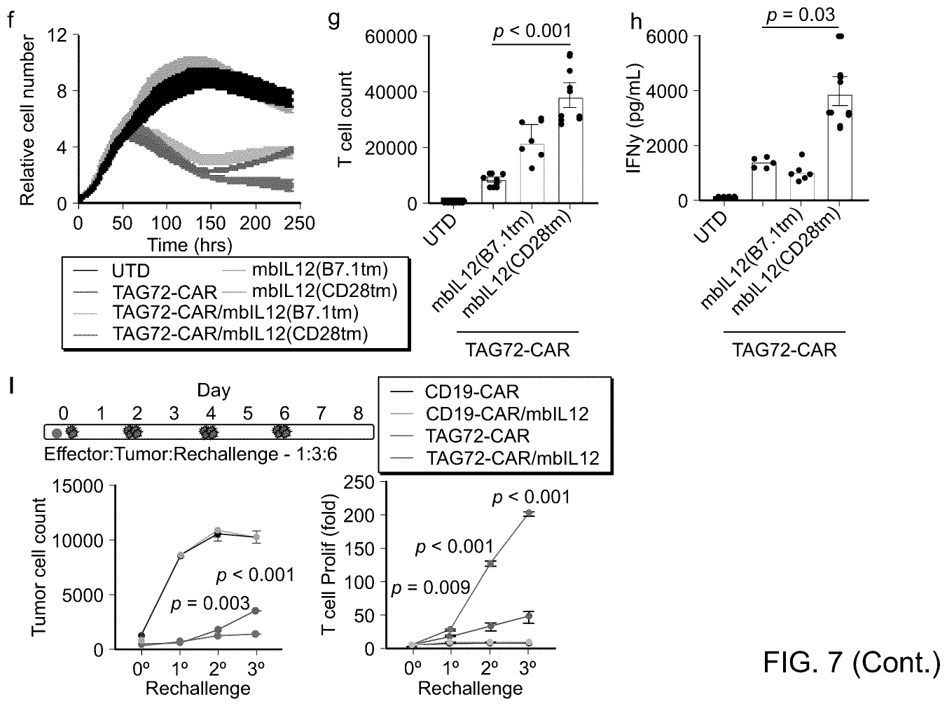

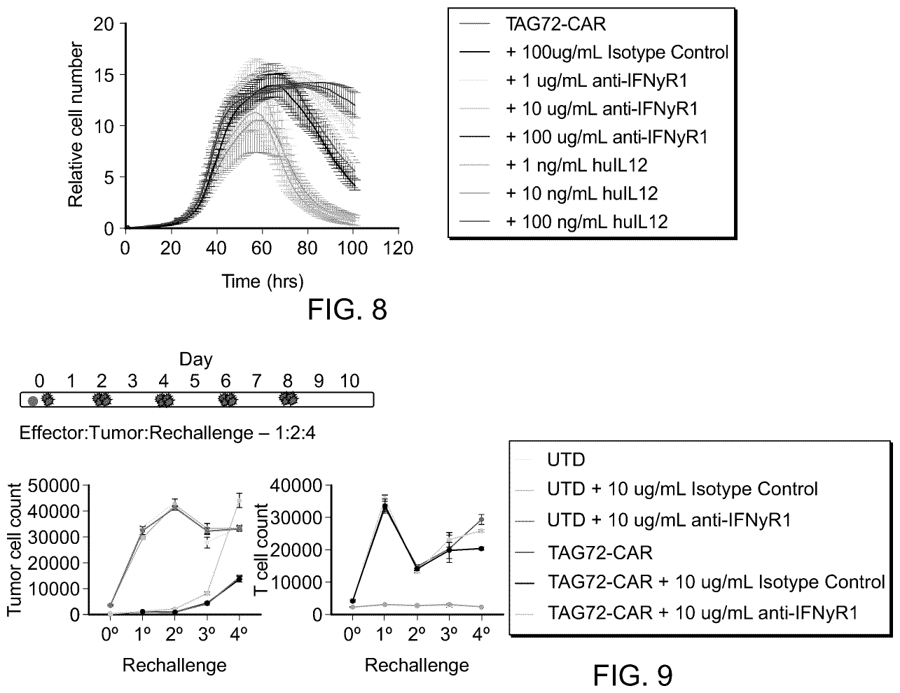

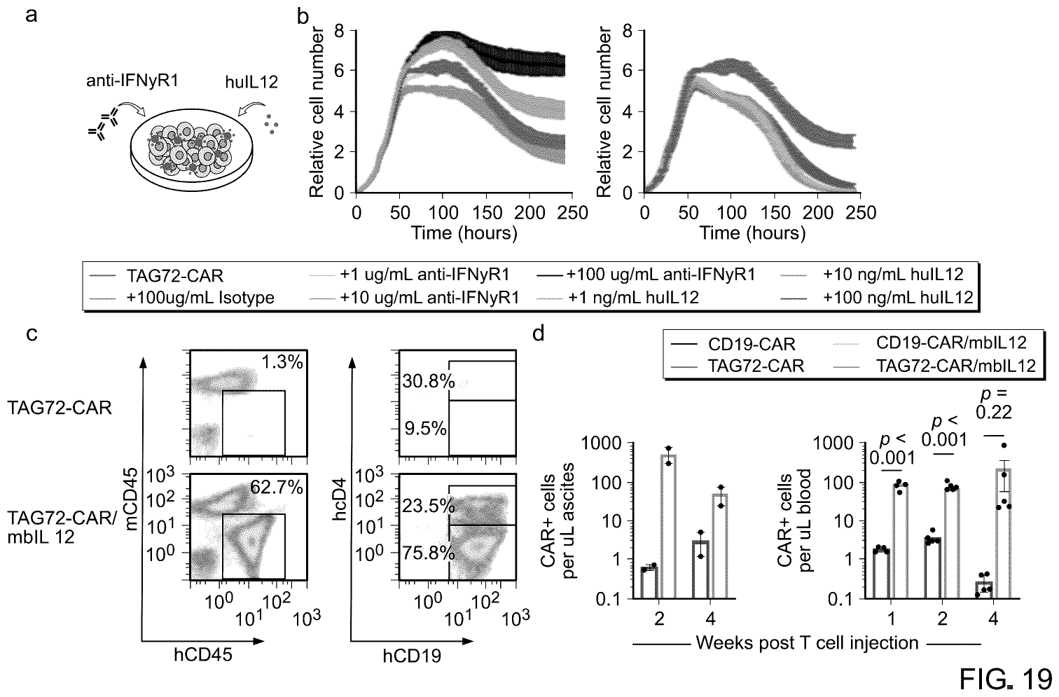

Based on accumulating evidence that IFNγ signaling impacts therapeutic activity of CAR T cells³⁹⁻⁴¹ along with our observation that TAG72-dCH2(28tm)BBz CAR T cells secreted the highest levels of IFNγ in our studies, we hypothesized that IFNγ signaling contributed to the superior anti-tumor activity of TAG72-dCH2(28tm)BBz CAR T cells. To test this hypothesis, we inhibited IFNγ signaling using an anti-IFNγR1 blocking antibody or enhanced IFNγ secretion with a human recombinant interleukin-12 (IL-12) in our extended in vitro coculture tumor cell killing assay (FIGS. 19a and 7a-c). Strikingly, we saw dose-dependent dampening of tumor cell killing with blockade of IFNγ signaling using OV90 tumors 234 cells (FIG. 19b, left and FIG. 7A) and OVCAR3 tumor cells (FIG. 8). We also observed dose-dependent enhancement of tumor cell killing with increasing concentrations of recombinant huIL12, which resulted in increased production of IFNγ by CAR T cells as determined by ELISA at the assay endpoint (FIG. 19b, right and 2c). We also observed dose-dependent enhancement of tumor cell killing with increasing concentrations of recombinant huIL-12, which resulted in the significant secretion of IFNγ by CAR T cells as determined by ELISA at the assay endpoint (FIG. 7b). Interestingly, only modest impact was observed in T cell proliferation following IFNγ blockade or addition of huIL-12 in this assay (FIG. 7c). We further corroborated our findings using a recursive tumor cell killing assay, showing a requirement of IFNγ in promoting sustained anti-tumor activity in vitro (FIG. 9).

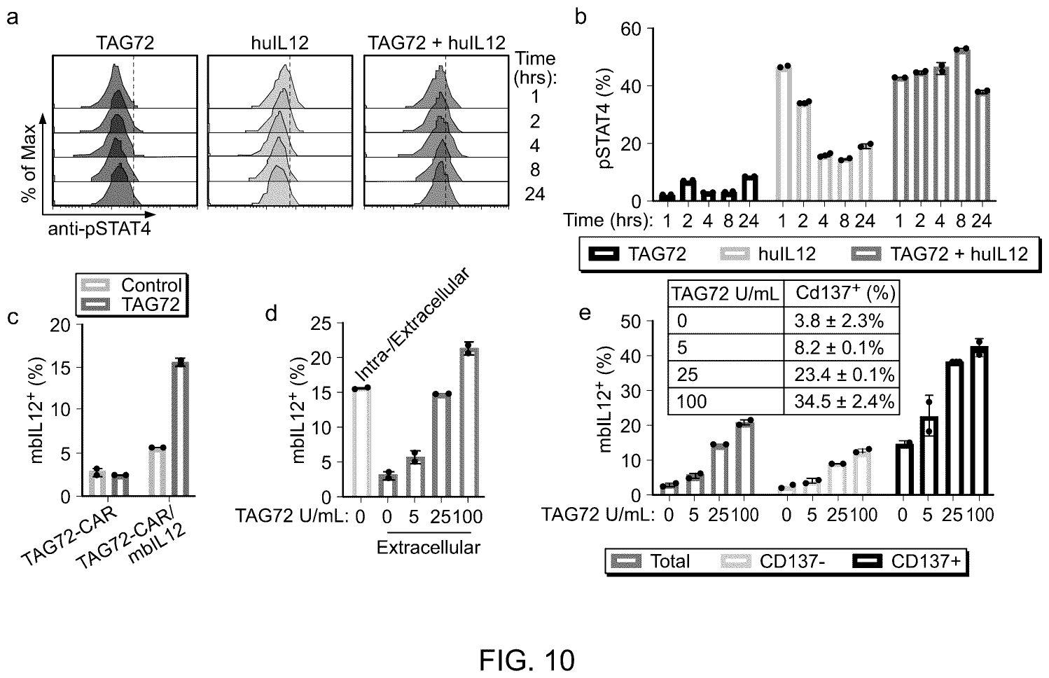

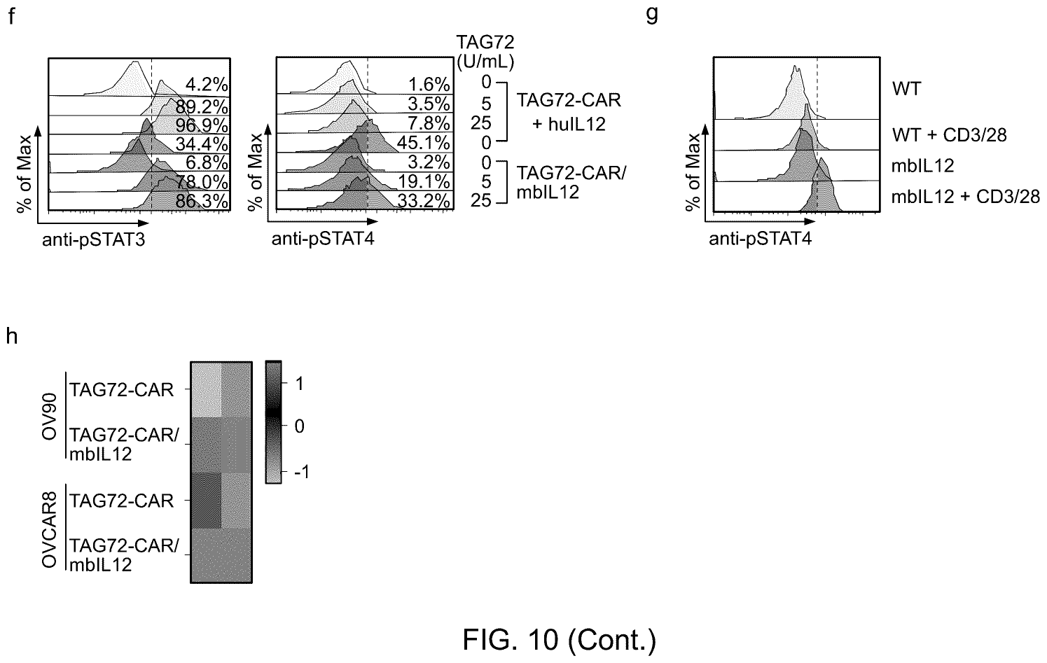

We sought to better understand the signaling kinetics downstream of IL-12 and in the context of CAR T cell antigen stimulation. We first optimized an assay to confirm phosphorylated STAT4 (pSTAT4) downstream of huIL12 in TAG72-CAR T cells. Interestingly, we observed that while pSTAT4 levels peaked at 1 hr and declined over the 24 hr time course with recombinant huIL12 alone; pSTAT4 was sustained over the 24 hr period in CAR T cells that were stimulated with plate-bound TAG72 antigen and huIL12 in combination (FIGS. 10A-10B). These data suggest that IL-12 signaling enhanced CAR T cell 247 activation and cytotoxicity, and conversely CAR T cell activation enhances persistent IL 2/pSTAT4 signaling

### Example 4: Engineered Membrane-Bound IL-12 Signaling Drives Robust CAR T Cell-Mediated Tumor Cell Killing and Antigen-Dependent T Cell Proliferation

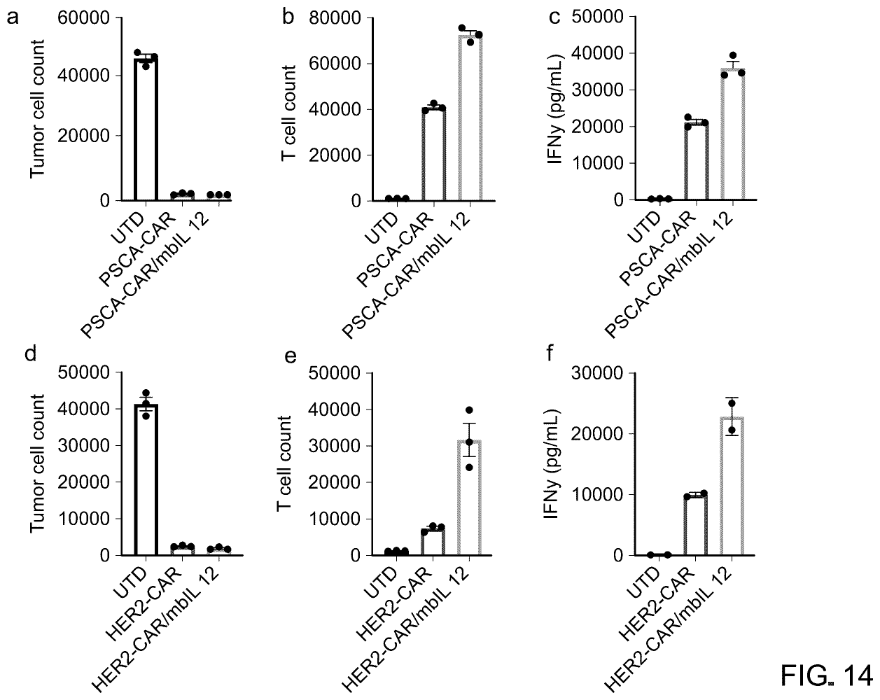

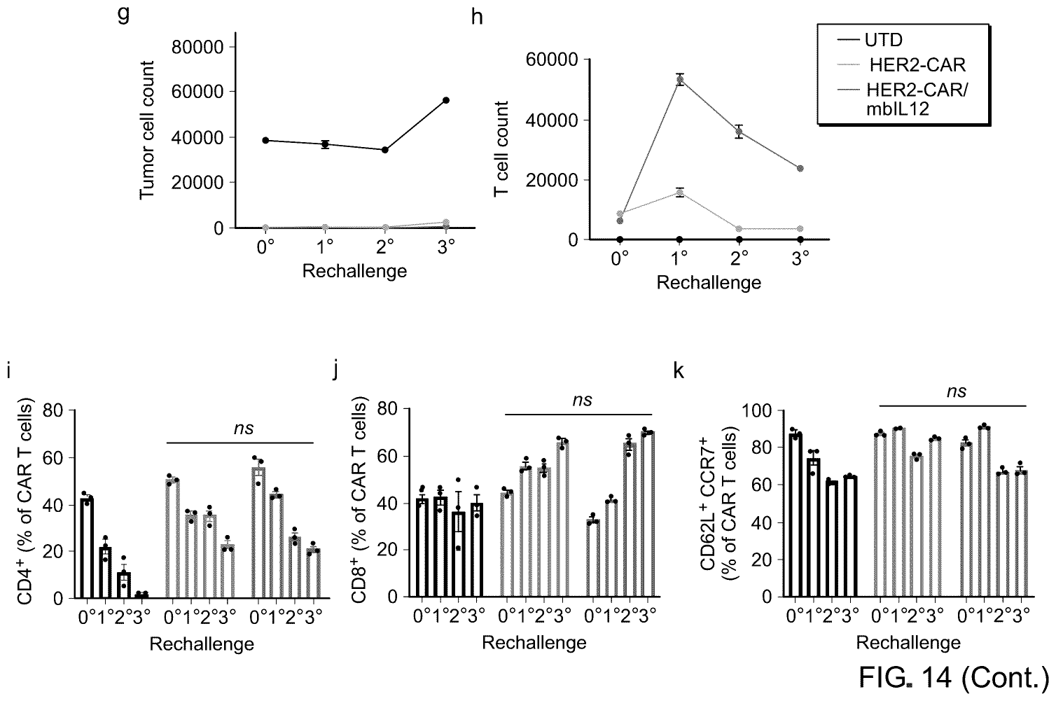

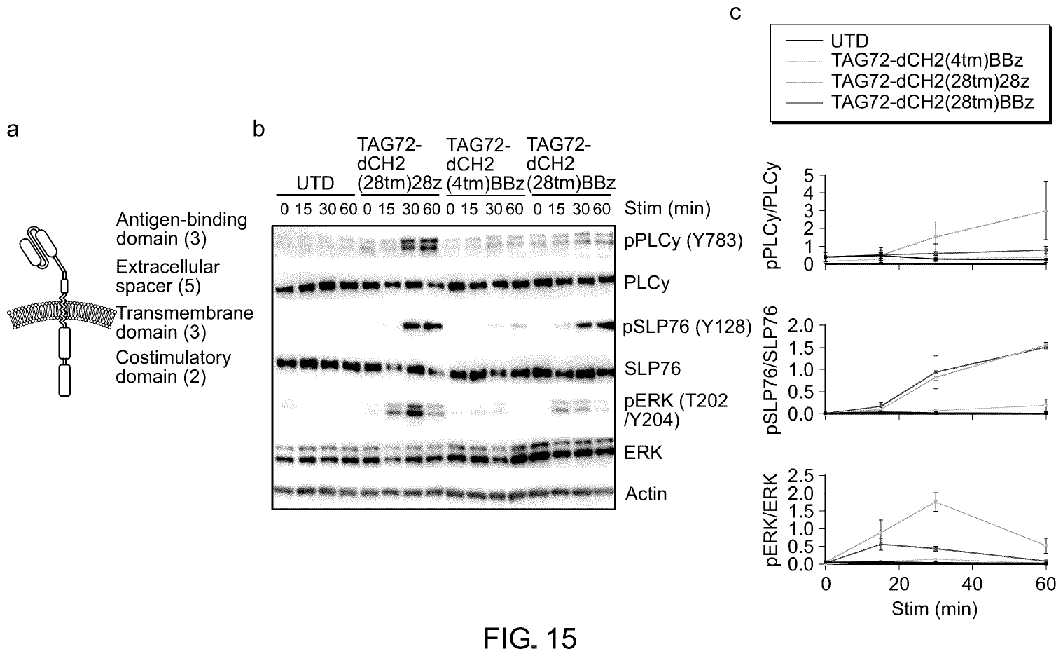

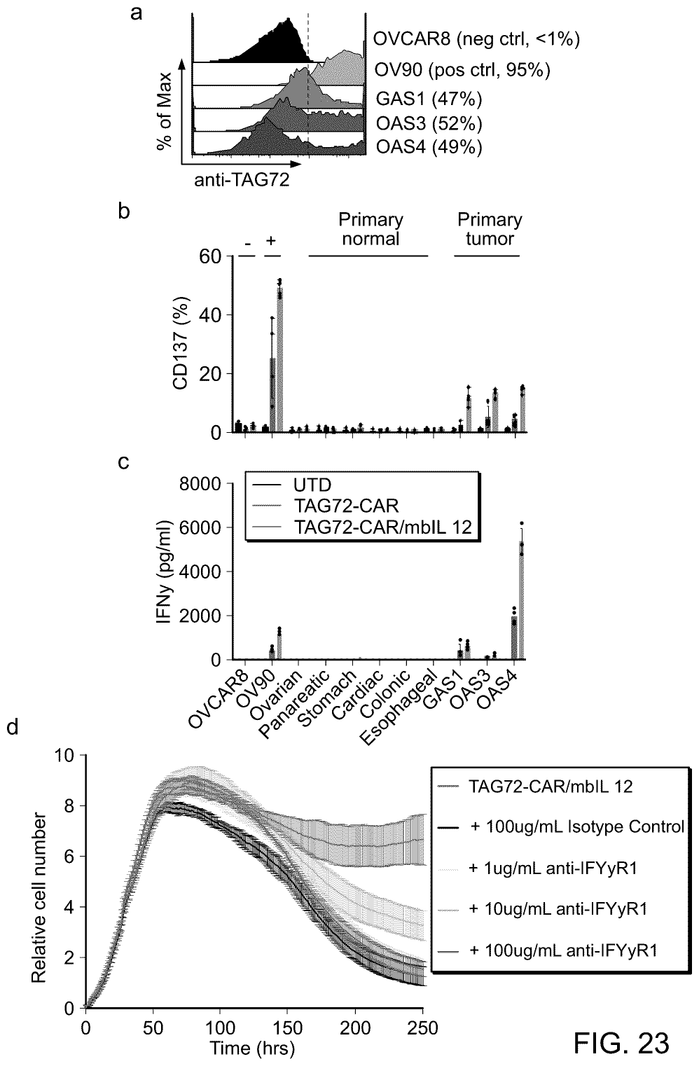

Building on our in vitro findings that IL-12-induced IFNγ signaling is critical for CAR T cell anti-tumor activity, we engineered IL-12 into our TAG72-CAR T cells. Due to the potential off-target toxicity induced by secreted IL-12⁴², we aimed to spatially restrict IL-12's effect by building membrane-bound IL-12 (mbIL12) constructs with varying transmembrane domains (FIG. 7d). When we checked for their cell surface expression, we observed slightly enhanced expression of mbIL12(CD28tm) (SEQ ID NO: 6 or 7) compared to mbIL12(B7.1tm), although both showed appreciable expression on TAG72-CAR T cells (FIG. 7e). We then evaluated tumor cell killing activity of TAG72-CAR T cells expressing either of the two versions of mbIL12, and found that TAG72-CAR/mbIL12(CD28tm) T cells displayed the highest tumor cell killing activity, T cell expansion and IFNγ production (FIG. 7f-7h). In these in vitro assays, the T cell cytotoxicity benefits with mbIL12 relied on IFNγ signaling (FIG. 23d). Similar enhancement of activation and IFNγ production with mbIL12 was shown against human patient-derived TAG72+ gastric and ovarian cancer ascites cells, with little to no impact on targeting of normal primary human cells (FIG. 23a-23c). T cell functional benefits were also observed using HER2- and PSCA-targeting CAR T cells engineered with mbIL12(CD28tm) (FIG. 14a-14h). mbIL12-engineered CAR T cells showed little/no changes in CD4/CD8 ratios, or markers of naïve/memory phenotypes (CD62L, 269 CCR7) in the in vitro functional assays (FIG. 14i-14k). We further assessed activity of CAR T cells with mbIL12(CD28tm) in recursive tumor cell killing, in which we again observed enhanced tumor cell killing and CAR antigen-dependent T cell expansion over multiple rechallenge time points (FIG. 7i).

We then compared the expression of cell-surface mbIL12 on TAG72-CAR T cells that were rested in the absence of serum or exogenous cytokines prior to stimulation with plate-bound TAG72 antigen or control antigen. Without T cell stimulation, cell-surface mbIL12 was detected at low levels on T cells, but was rather found largely intracellularly (FIGS. 10c-10d). We showed comparable mRNA transcripts of mbIL12 in stimulated and non-stimulated T cells, suggesting that low cell-surface mbIL12 expression was likely due to subcellular localization and not driven by differential EF1alpha promoter activity (FIG. 10h). Interestingly, significant dose-dependent increases in cell-surface expression of mbIL12 was observed in antigen-stimulated CAR T cells, in particular in CD137+-activated T cell subsets (FIG. 10d-10e).

### Example 5: Antigen-Dependent IL-12 Signaling in CAR T Cells

We sought to better understand the signaling kinetics downstream of IL-12 with phosphoflow cytometry. We first optimized an assay to confirm phosphorylated STAT4 (pSTAT4) downstream of recombinant human IL-12 (huIL12) in TAG72-CAR T cells. Interestingly, we observed that while pSTAT4 levels peaked at 1 hr and declined over the 24 hr timecourse with recombinant huIL12 alone, pSTAT4 was sustained over the 24 hr period in CAR T cells that were stimulated with plate-bound TAG72 antigen and huIL12 in combination (FIG. 10a-b). We then compared the expression of cell surface mbIL12 on TAG72-CAR T cells that were rested in the absence of serum or exogenous cytokines prior to stimulation with plate-bound TAG72 antigen or control antigen. We observed that without stimulation, mbIL12 was detected at very low levels in engineered T cells. However, a significant increase in cell surface expression of mbIL12 was seen in antigen-stimulated TAG72-CAR T cells (FIG. 10c). Next, we interrogated pSTAT4 expression in TAG72-CAR/mbIL12 T cells in response to CAR stimulation. We observed the expected phosphorylation of STAT3 in response to TAG72 antigen in both TAG72-CAR and TAG72-CAR/mbIL12 T cells, which was only slightly activated by huIL12. However, CAR stimulation showed dose-dependent increases in pSTAT4 in TAG72-CAR/mbIL12 T cells compared to TAG72-CAR T cells alone (FIG. 10d). We further evaluated the potential for trans signaling in TAG72-CAR/mbIL12 T cells. We transduced HT1080 (TAG72-) cells with mbIL12 and cocultured them with T cells in the presence of soluble CD3/CD28 stimulation. Increased pSTAT4 in T cells was observed when cultured with both HT1080-mbIL12 and soluble CD3/CD28 and not with HT1080-mbIL12 alone or HT1080-WT with CD3/CD28 (FIG. 10e). Collectively, these data suggest that mbIL12 engineered CAR T cells demonstrate improved in vitro anti-tumor activity and unexpectedly rely on CAR antigen stimulation, which we termed antigen-dependent IL-12 signaling.

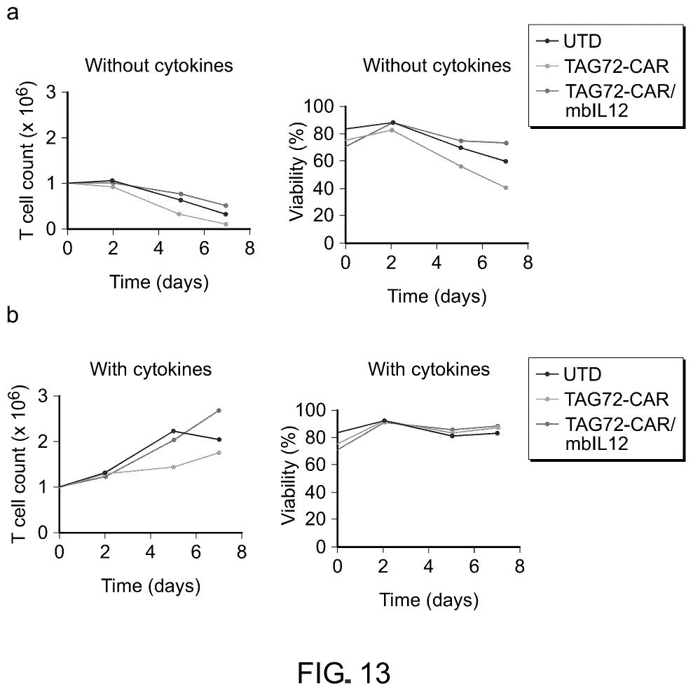

Next, we interrogated downstream pSTAT4 expression in TAG72-CAR/mbIL12 T cells in response to CAR stimulation. We observed the expected phosphorylation of STAT3 in response to TAG72 antigen in both TAG72-CAR and TAG72-CAR/mbIL12 T cells, which was only slightly activated by huIL12. However, CAR stimulation showed dose-dependent increases in pSTAT4 in TAG72-CAR/mbIL12 T cells compared to TAG72-CAR T cells alone (FIG. 1f). We further evaluated the potential for trans signaling in TAG72-CAR/mbIL12 T cells. We transduced HT1080 (TAG72-) cells with mbIL12 and co-cultured them with T cells in the presence of soluble CD3/CD28 stimulation. Increased pSTAT4 in T cells was observed when cultured with both HT1080-mbIL12 and soluble CD3/CD28 and not with HT1080-mbIL12 alone or HT1080-WT with CD3/CD28 (FIG. 10g). Anti-tumor activity was further assessed in recursive tumor cell killing assays, in which we again observed enhanced tumor cell killing and CAR antigen-dependent T cell expansion over multiple rechallenge timepoints in CAR T cells engineered with mbIL12 (FIG. 7i). Importantly, no expansion or survival benefits were observed in the absence of CAR antigen stimulation (FIGS. 13a-13b). Collectively, these data suggest that mbIL12-engineered CAR T cells demonstrate improved in vitro anti-tumor activity and unexpectedly rely on CAR antigen stimulation, which we termed antigen-dependent IL-12 signaling.

### Example 6: Superior Anti-Tumor Activity by Antigen-Dependent IL-12 Signaling in CAR T Cells

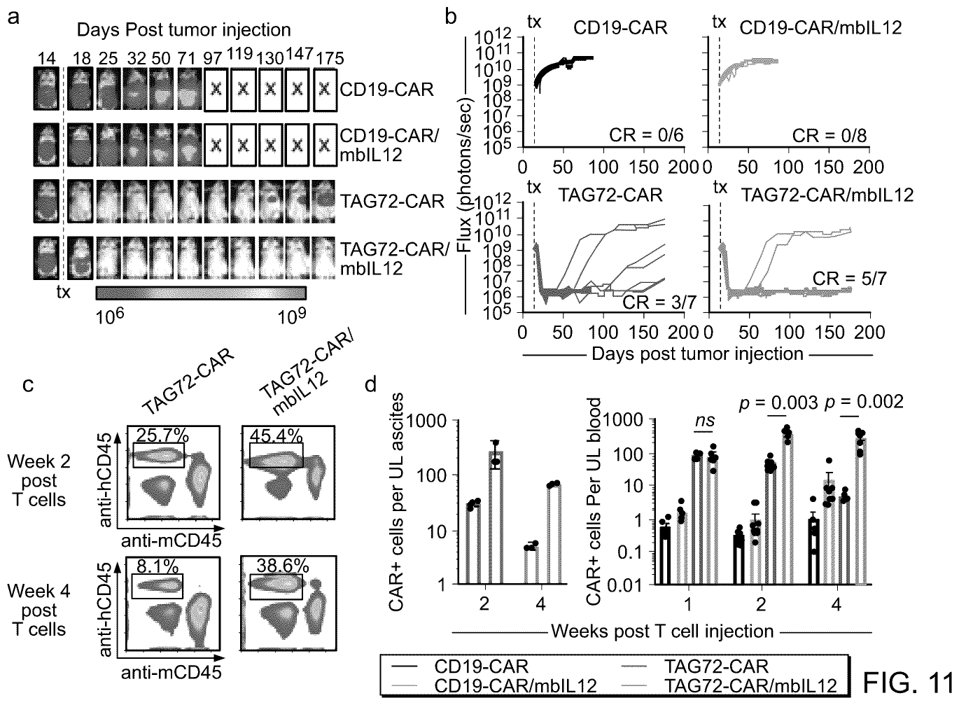

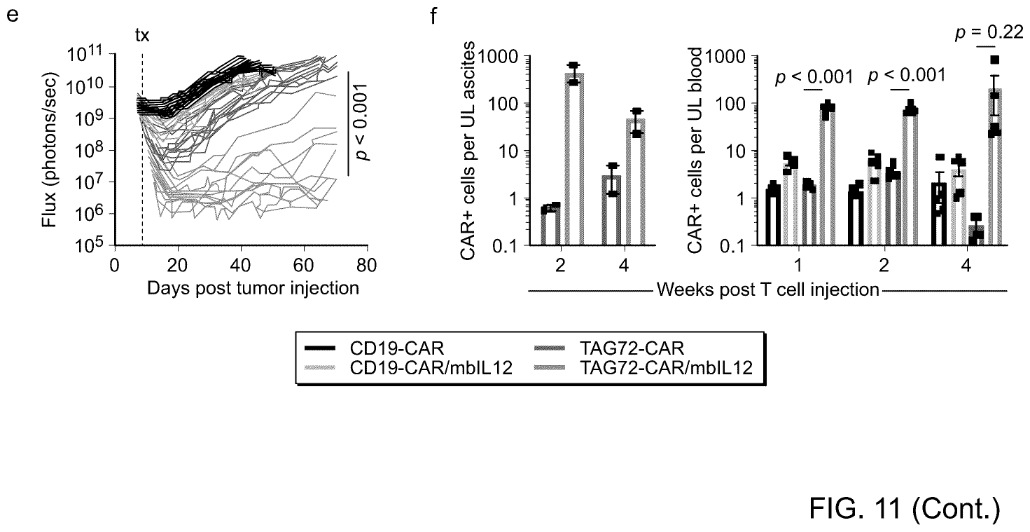

We next evaluated the therapeutic potential of TAG72-CAR T cells with antigen-dependent IL-12 signaling. Using the i.p. OVCAR3 tumor xenograft model, mice treated with TAG72-CAR/mbIL12 T cells sustained more durable anti-tumor responses as compared to TAG72-CAR T cells, achieving a greater incidence of durable complete responses (FIG. 11a-b). Importantly, tumor-bearing mice treated with CD19-CAR T cells and CD19-CAR/mbIL12 T cells showed little differences in therapy, supporting the antigen-dependent nature of mbIL12. We observed a higher frequency of hCD45+ cells in the peritoneal ascites of mice treated with TAG72-CAR/mbIL12 T cells as compared to mice treated with TAG72-CAR T cells alone at 2 and 4 weeks post-treatment (FIGS. 11c-11d and 19c-19d). We also observed significantly higher and sustained levels of TAG72-CAR/mbIL12 T cells in peripheral blood as compared with TAG72-CAR T cells alone (FIGS. 11d and 19d). We replicated these findings using the more challenging i.p. OV90 tumor xenograft model, where the differences in therapy and T cell persistence between TAG72-CAR and TAG72-CAR/mbIL12 T cells were even more pronounced (FIG. 11e-f).

### Example 7: Improved Systemic Disease Targeting by CAR T Cells with Antigen-Dependent IL-12 Signaling

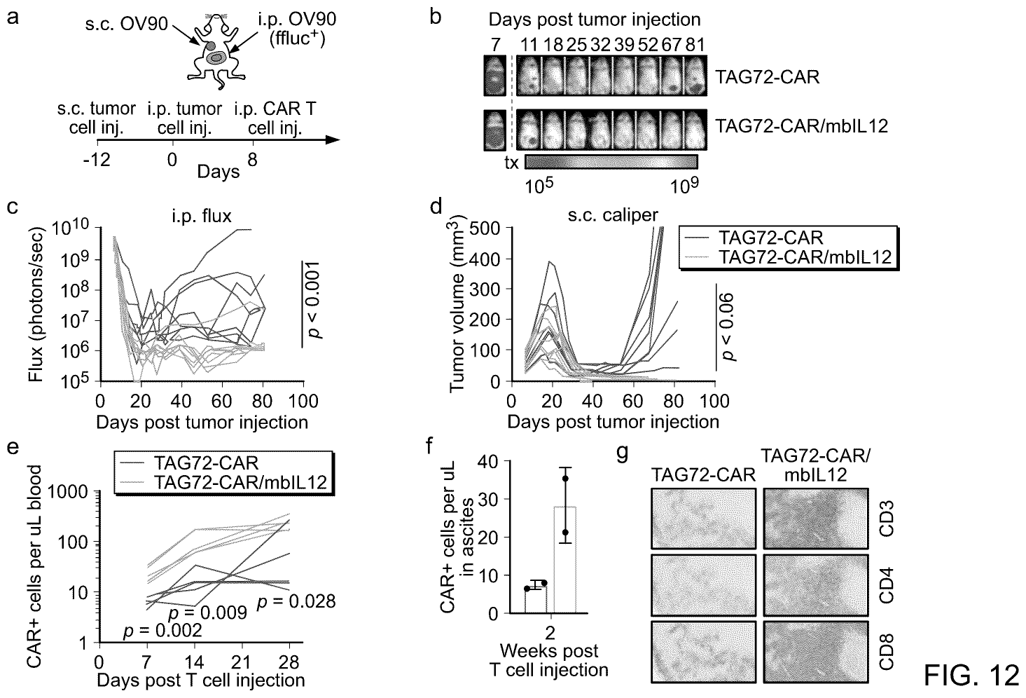

One prevailing argument against the locoregional administration of CAR T cells is their potential spatial confinement, thereby preventing systemic therapy in patients with widespread metastatic disease⁴³. However, our data suggest that regional intraperitoneally-administered mbIL12-engineered CAR T cells may have a greater capacity to target disease outside of the peritoneum. To test this, we established a xenograft OV90 tumor model with both a regional i.p. and systemic s.c. tumor in the same mouse. OV90 (ffluc-negative) tumor cells were injected subcutaneously (s.c.) to track with calipers measurement, and OV90 (eGFP/ffluc-expressing) tumor cells were i.p. injected and tracked with bioluminescent flux imaging (FIG. 12a). As we observed in previous experiments, i.p. anti-tumor responses were greater in mice regionally treated with TAG72-CAR/mbIL12 T cells as compared to TAG72-CAR T cells alone (FIG. 12b-c). While s.c. tumors initially regressed similarly in both treatment groups, all tumors recurred following TAG72-CAR T cell treatment alone, whereas s.c. tumors were completely eradicated in all mice followingTAG72-CAR/mbIL12 T cell treatment (FIG. 12d). We corroborated this phenomenon by again observing higher levels of CAR T cells in the peripheral blood and peritoneal ascites of mice treated with TAG72-CAR/mbIL12 T cells (FIG. 12e-f). Furthermore, immunohistochemistry (IHC) analysis of s.c. tumors at day 12 post treatment demonstrated significantly greater infiltration of TAG72-CAR/mbIL12 T cells as compared with TAG72-CAR T cells alone (FIG. 12g). Overall, these data support logoregional delivery of CAR T cells with engineered antigen-dependent IL-12 signaling in durable targeting both regional and systemic disease.

### Example 8: Membrane-Bound IL-12 Improves Anti-Tumor Activity of Various CAR

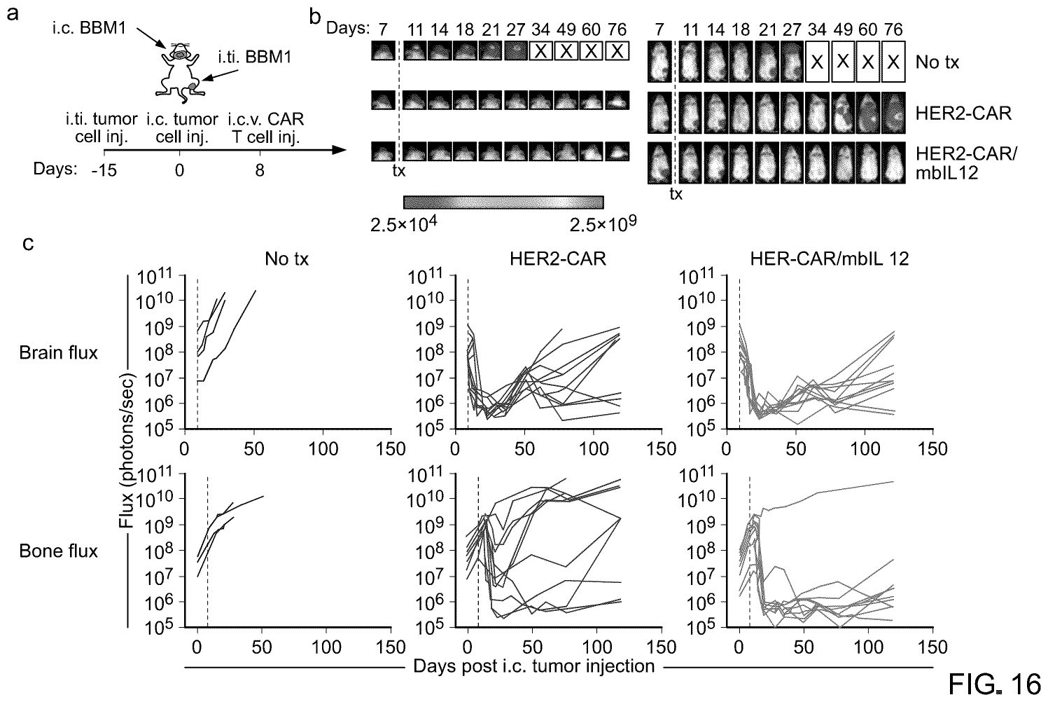

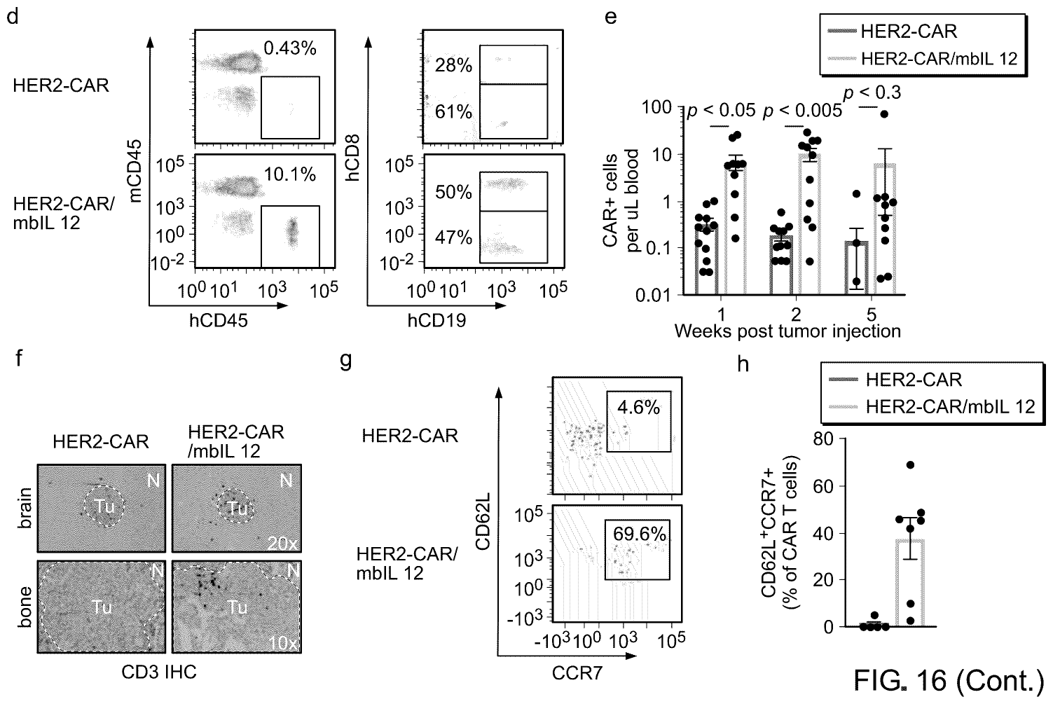

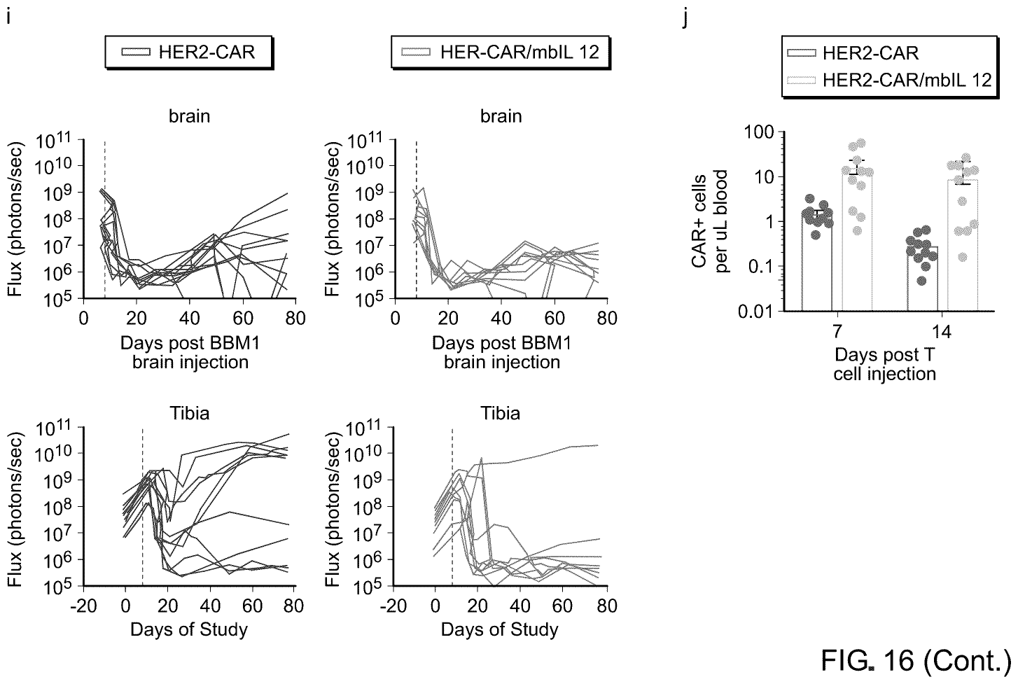

We further engineered CAR T cells targeting HER2 or PSCA, with a membrane-bound IL-12 molecule mbIL12(CD28tm) (SEQ ID NO:F) and showed similar improvements in anti-tumor activity (FIG. 14a-f). To extend our finding that mbIL12 signaling enhances regional to systemic disease targeting by CAR T cells, we used the patient-derived HER2+ BBM1 tumor model we previously published in developing our HER2-CAR T cells. Using this model system, we established a BBM1 tumor xenograft model with both regional intracranial (i.e.) brain metastasis and intratibial (i.ti.) bone metastasis in the same mouse. BBM1-ZsGreen-ffluc tumor cells were i.e. and i.ti. injected and tracked with bioluminescent flux imaging (FIG. 16a). Mice were treated with a single dose of HER2-CAR T cells by regional intracerebroventricular (i.c.v.) delivery corresponding to day 8 post i.e. tumor injection and day 23 post i.ti. tumor injection. We observed potent therapeutic responses in the brain using either HER2-CAR T cells alone or HER2-CAR T cells engineered with mbIL12 (FIGS. 16b and 16c, top row). However, only mbIL12-engineered HER2-CAR T cells demonstrated curative responses in i.ti. bone metastases in the majority of treated mice, compared with heterogeneous responses with HER2-CAR T cells alone (FIGS. 16b and 16c, bottom row). Similar to our observations in the dual-tumor ovarian cancer model above, we observed significant expansion and greater persistence of HER2-CAR/mbIL12 T cells in the peripheral blood of regional i.c.v. treated mice, as compared with HER2-CAR T cells alone (FIG. 16d-16e), with persisting HER2-CAR/mbIL12 T cells in the blood showing a greater central memory phenotype (FIG. 16g-16h), along with greater T cell infiltration in both brain and bone metastases (FIG. 16f). Collectively, these two models strongly support the benefits of mbIL12 in targeting systemic disease following regional administration of CAR T cells.

### Example 9: mbIL12-Engineered CAR T Cells Demonstrate Safety and Efficacy in an Immunocompetent Mouse Model of Ovarian Cancer Peritoneal Metastasis

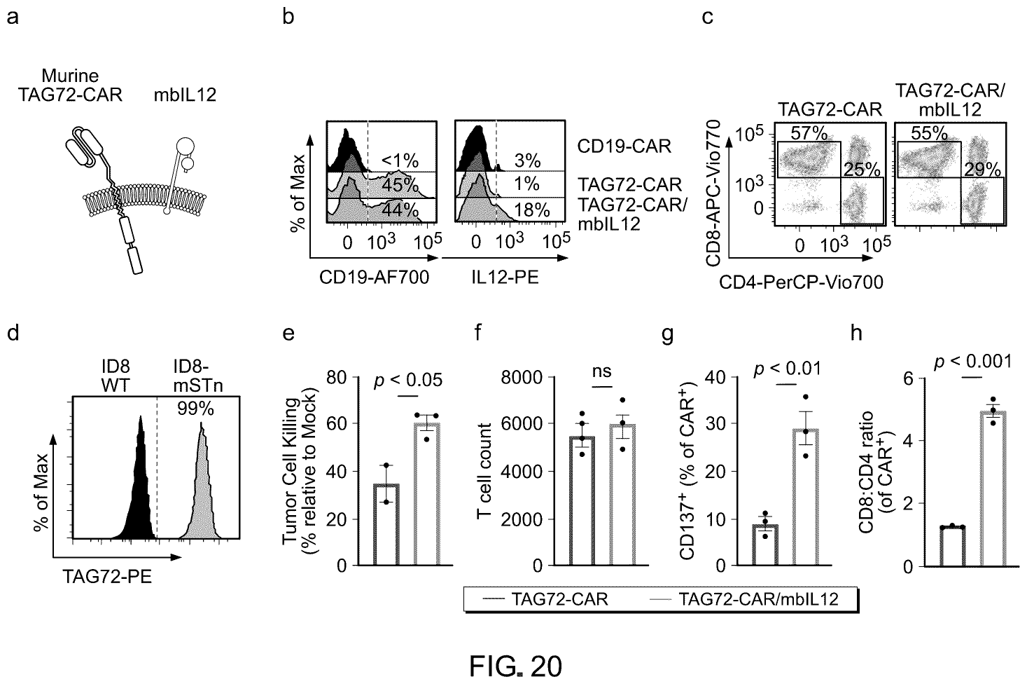

The safety concerns of IL-12 have limited its therapeutic applications in humans to date. Infusion of soluble recombinant IL-12 in patients has resulted in unwanted toxicities, including colitis. Additionally, engineering adoptive T cell therapies with soluble IL-12, even under NFAT inducible promoter systems to limit IL-12 production to activated T cells has resulted in similar toxicities. To test whether mbIL12-engineered CAR T cells is a safe therapeutic approach compared with soluble IL-12, we built a fully immunocompetent mouse model of TAG72+ ovarian cancer peritoneal metastasis, along with a murine version of our TAG72-CAR and mbIL12 constructs (FIG. 20a). TAG72-CAR T cells were efficiently manufactured as previously described using retrovirus transduction of murine splenic T cells, and were further engineered to express mbIL12 (FIG. 20b-20c). Murine ID8 ovarian cancer cells were stably transduced and cloned to express TAG72 using the mouse sialyl-transferace (mSTn) were used as tumor targets for our studies (FIG. 20d). Murine TAG72-CAR/mbIL12 T cells demonstrated increased activity and cytotoxicity against TAG72+ ID8 tumor cells, as compared with TAG72-CAR T cells alone (FIG. 20e-20g). We observed a bias towards antigen-dependent expansion of CD8+ CAR T cells, relative to the starting product, during the tumor cell killing assays in vitro (FIGS. 20c and 20h).

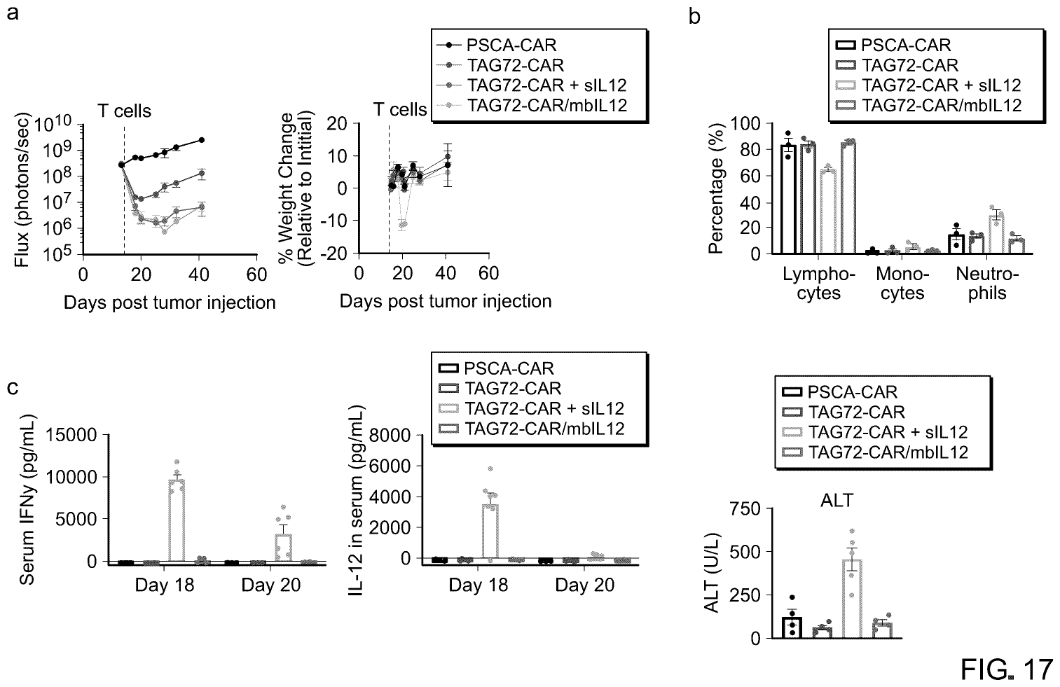

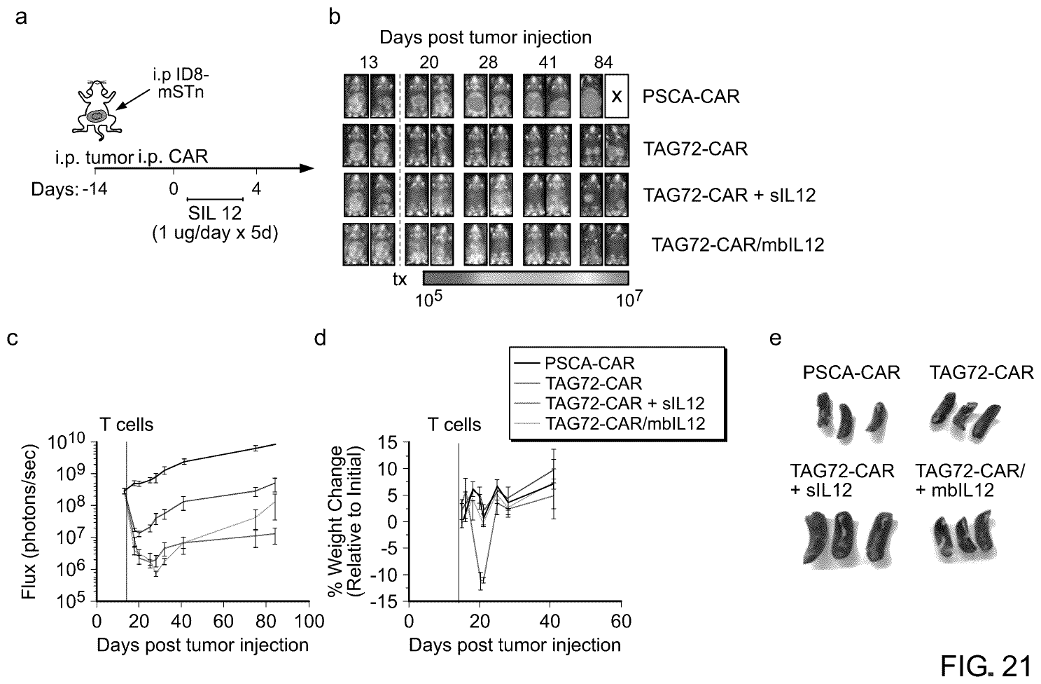

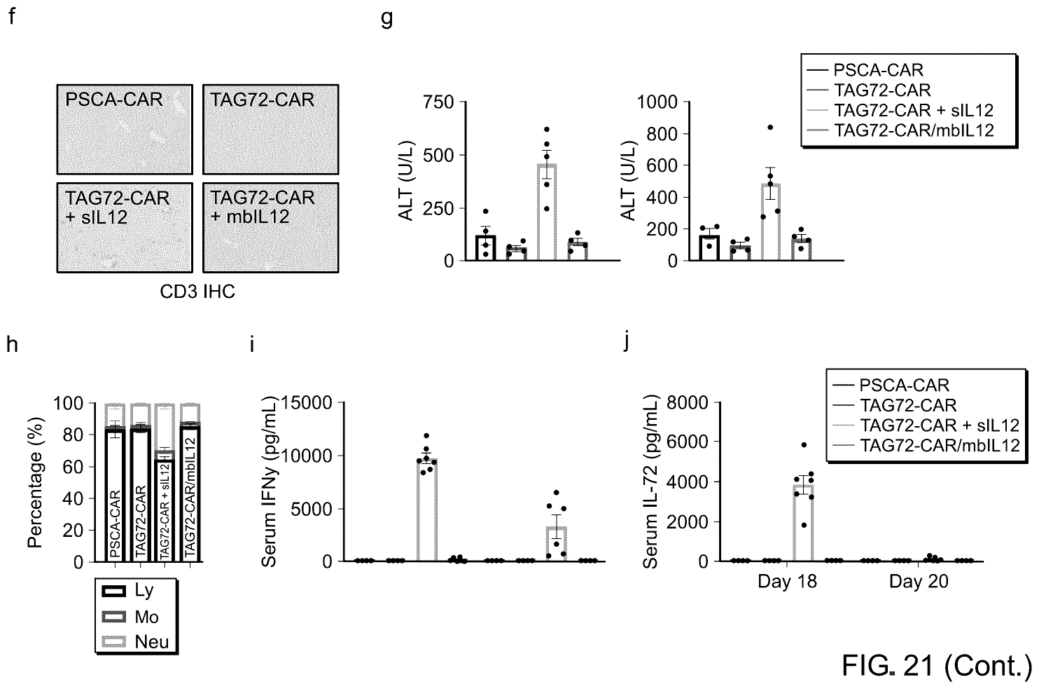

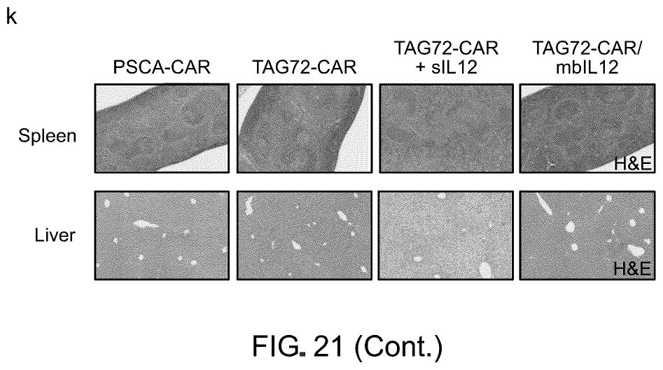

We performed a head-to-head safety and efficacy comparison of CAR T cells either engineered with mbIL12 or in combination with soluble IL-12 injections. TAG72+ ID8 (ffluc-positive) tumor cells were injected i.p. and tracked with bioluminescent flux imaging, and on day 14 post tumor injection were regionally i.p. treated with either TAG72-CAR T cells alone, TAG72-CAR/mbIL12 T cells, or TAG72-CAR T cells along with soluble IL-12 (sIL12) injections for 5 days (FIG. 21a). Potent therapeutic responses were seen in all treated mice, but were greater in mice regionally treated with TAG72-CAR/mbIL12 T cells or TAG72-CAR T cells with slL12, as compared to TAG72-CAR T cells alone (FIGS. 17a, 21b-21c). Interestingly, at later time points, we observed recurrences in mice treated with CAR T cells and sIL12 injections, but durable tumor control in mice treated with CAR T cells engineered with mbIL12. While IL-12 benefited CAR T cell therapy by injection or with T cell engineering, we observed significant body weight loss in mice following TAG72-CAR T cells and sIL12 injections, and not in TAG72-CAR/mbIL12 treated mice (FIGS. 17a, 21d). We confirmed systemic toxicities associated with sIL12 injections, with mice showing signs of splenomegaly (FIGS. 21e, 21k, top), signs of liver abnormalities and greater T cell infiltration in the liver (FIGS. 21f and 21k, bottom), along with increased serum ALT and AST levels and changes in peripheral blood lymphocyte counts (FIGS. 17b, 21g-21h), while no appreciable systemic effects were observed with TAG72-CAR/mbIL12 T cells. Importantly, slL12 injections resulted in increases in systemic IFNγ levels in serum of TAG72-CAR T cell treated mice, which was undetectable in TAG72-CAR/mbIL12 treated mice (FIGS. 17c, 21i-21j). These data support the safety and efficacy of regionally administered mbIL12-engineered CAR T cells in an immunocompetent mouse tumor model.

### Example 10: mbIL12-Engineered CAR T Cells Modify the Immunosuppressive Tumor Microenvironment in Peritoneal Metastasis

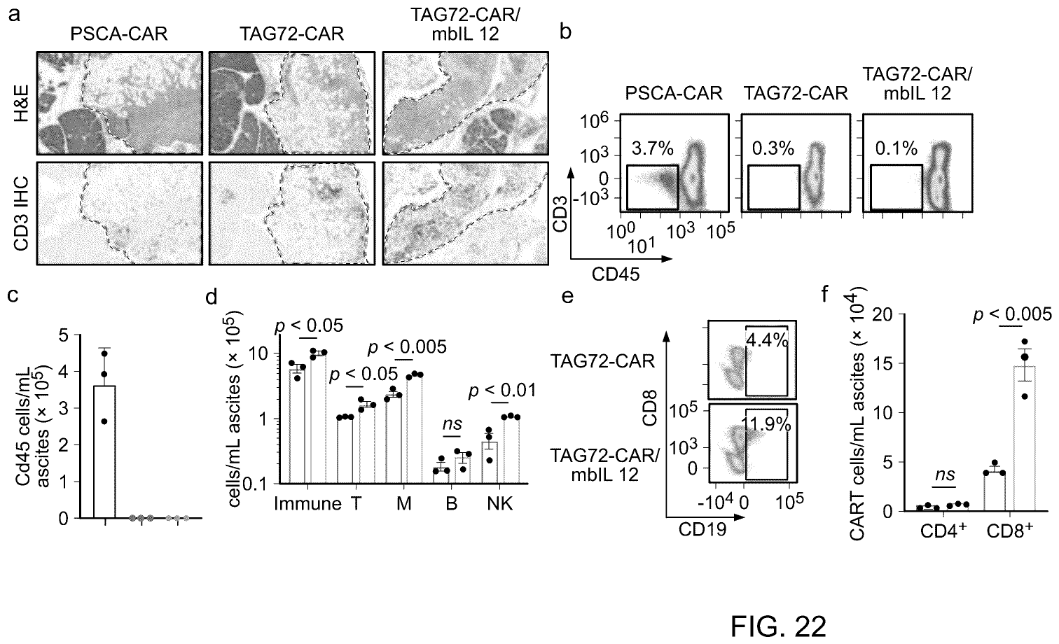

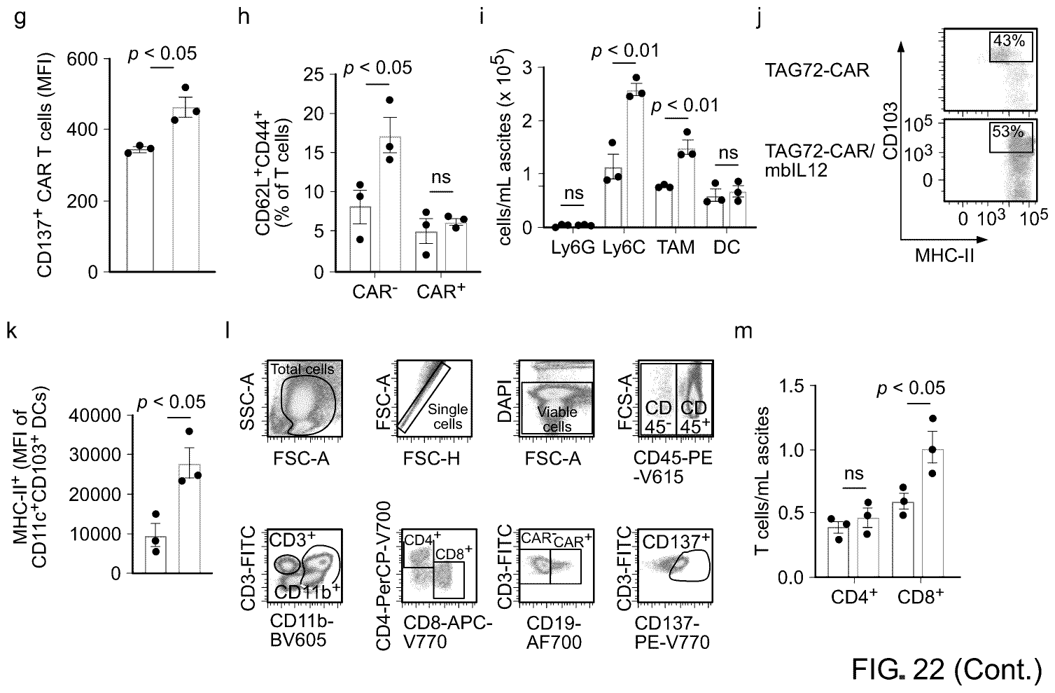

IL-12 has numerous effects on the tumor microenvironment, including reshaping the myeloid cell compartment, promoting antigen-presentation, and enhancing adoptive T cell therapies. We therefore assessed the impact of mbIL12-engineering of CAR T cells and its role in the tumor microenvironment in the ovarian cancer peritoneal metastasis model. Peritoneal tumors harvested from mice treated with TAG72-CAR/mbIL12 T cells showed increased T cell infiltration as compared with TAG72-CAR T cells alone (FIG. 22a). At this early timepoint post therapy, both treated groups showed comparable potency in clearing tumor cells in the peritoneal ascites (FIGS. 22b-22c). We further evaluated the peritoneal ascites for changes in immune cell subsets by flow cytometry (FIG. 22l), which showed increases in overall CD45+ immune cell counts, comprised of T cells, myeloid cells, and NK cells, with little change in total B cell counts (FIGS. 22d and 22m). TAG72-CAR T cells engineered with mbIL12 showed increased persistence of CD8+ T cells in the peritoneal ascites (FIGS. 22e-22f), and a small but significant increase in CD137 activation (FIG. 22g). Interestingly, while TAG72-CAR/mbIL12 T cells persisting showed little change in CD62L+CD44+ naïve/memory phenotypes, there was an increase in naïve/memory phenotypes of non-CAR T cells in the peritoneal ascites (FIG. 22h). We then evaluate changes in the myeloid cell compartment in the peritoneal ascites, showing significant increases in total 17 F4/80+ tumor-associated macrophages (TAM), along with increases in Ly6C+ inflammatory monocytes, without changes in Ly6G+ neutrophil and CD11c+DC populations (FIG. 22i). However, we observed significant increases in MHC-II expression among the CD11c+CD103+414 DCs, suggesting an improved mature antigen-presenting phenotype. Collectively, these findings demonstrate beneficial modulation of the immunosuppressive tumor microenvironment in this model by mbIL12-engineered CAR T cells

### Materials and Methods Used in Examples

### Cell Lines

The epithelial ovarian cancer line OVCAR3 (ATCC HTB-161) was cultured in RPMI-1640 (Lonza) containing 20% fetal bovine serum (FBS, Hyclone) and 1× antibiotic-antimycotic (1×AA, Gibco) (complete RPMI). The epithelial ovarian cancer line derived from metastatic ascites OV90 (CRL-11732) was cultured in a 1:1 mixture of MCDB 105 medium (Sigma) and Medium 199 (Thermo) adjusted to pH of 7.0 with sodium hydroxide (Sigma) and final 20% FBS and 1× AA. The epithelial ovarian cancer line OVCAR8 was a generous gift from Dr. Carlotta Glackin at City of Hope and was cultured in complete RPMI-1640. All cells were cultured at 37° C. with 5% CO₂. The breast-to-brain metastasis patient-derived line BBM1 was cultured as previously described⁶⁴. The mouse ovarian cancer cell line ID8 was cultured in Dulbecco's Modified Eagles Medium (DMEM) supplemented with 10% FBS, 2 mM L-Glutamine (Fisher Scientific), and 25 mM HEPES (Irvine Scientific) (cDMEM). The human breast cancer cell line MDA-MB-468 (ATCC HTB-132) was engineered to express HER2 (Accession: NM_004448.4) under the control of the EF1a promotor via epHIV7 lentivirus transduction (468-HER2). All cells were cultured at 37° C. with 5% CO2. Human primary cell lines were obtained from Cell Biologics (Human Primary Colonic Epithelial Cells H-6047, Human Primary Esophageal Epithelial Cells H-6046, Human Primary Kidney Epithelial Cells H-6034, Human Primary Ovarian Epithelial Cells H-6036, Human Primary Pancreatic Epithelial Cells H-6037, Human Primary Proximal Tubular Epithelial Cells H-6015, Human Primary Small Intestine Epithelial Cells H-6051, Human Primary Stomach Epithelial Cells H-6039), Promocell (Human Cardiac Myocytes C-12810), and Lonza (Human Bronchial Epithelial Cells CC-2541) and cultured according to vendor's specifications.

### DNA Constructs, Tumor Lentiviral Transduction, and Retrovirus Production

Tumor cells were engineered to express enhanced green fluorescent protein and firefly luciferase (eGFP/ffluc) by transduction with epHIV7 lentivirus carrying the eGFP/ffluc fusion under the control of the EF1a promoter as described previously²³. The humanized scFv sequence used in the CAR construct was obtained from a monoclonal antibody clone huCC49 that targets TAG72³¹. The extracellular spacer domain included the 129-amino acid middle-length CH2-deleted version (ΔCH2) of the IgG4 Fc spacer³¹. The intracellular co-stimulatory signaling domain contained was a 4-1 BB with a CD4 transmembrane domain. The CD3ζ cytolytic domain was previously described³¹. Variations in extracellular spacer domains, transmembrane domains, and intracellular co-stimulatory signaling domains were described previously^{11,23,38}. The CAR sequence was separated from a truncated CD19 gene (CD19t) by a T2A ribosomal skip sequence, and cloned in an epHIV7 lentiviral backbone under the control of the EF1a promoter. The PSCA-BBζ CAR construct was described previously³⁸. The membrane-bound IL-12 (mbIL12) construct was generated using the p35 and p40 genes (p35, NC_000003.12; p40, NC_000005.10) separated by a G4S spacer (SEQ ID NO: 14), and linked to either the B7.1 or CD28 transmembrane domain. Lentivirus was generated as previously described³⁸. Lentiviral titers were quantified using HT1080 cells based on CD19t or IL-12 cell surface expression using flow cytometry. Human and murine tumor cells were engineered to express enhanced green fluorescent protein and firefly luciferase (eGFP/ffluc), or ffluc alone, by transduction with epHIV7 lentivirus carrying the eGFP/ffluc fusion or ffluc alone under the control of the EF1α promoter as described previously²³. Murine ovarian cancer cell line ID8 was also engineered to express target antigen TAG72 via transduction with epHIV7 lentivirus carrying the murine st6galnac-I gene (mSTn) under the control of the EF1α. mSTn is the unique sialyltransferase responsible for generating surface expression of aberrant glycosylation sialyl-Tn (TAG72). The humanized scFv sequence used in the CAR construct was obtained from a monoclonal antibody clone huCC49 that targets TAG7231

The extracellular spacer domain included the 129-amino acid middle-length CH2-deleted version (ΔCH2) of the IgG4 Fc spacer³¹. The intracellular co-stimulatory signaling domain contained was a 4-1 BB with a CD4 transmembrane domain. The CD3ζ cytolytic domain was previously described³¹. Variations in extracellular spacer domains, transmembrane domains, and intracellular co-stimulatory signaling domains were described previously^{11,23,38}. The CAR sequence was separated from a truncated CD19 gene (CD19t) by a T2A ribosomal skip sequence, and cloned in an epHIV7 lentiviral backbone under the control of the EF1α promoter. The PSCA-BBζ CAR and HER2-BBζ CAR constructs were described previously³⁸. The membrane-bound IL-12 (mbIL12) construct was generated using the p35 and p40 genes (p35, 532 NC_000003.12; p40, NC_000005.10) separated by a G4S spacer (SEQ ID NO: 14), and linked to either the B7.1 or CD28 transmembrane domain. Lentivirus was generated as previously described³⁸. Lentiviral titers were quantified using HT1080 cells based on CD19t or IL-12 cell surface expression using flow cytometry.

The scFv sequence from the mouse anti-human TAG72 antibody clone (CC49) was used to develop the murine CAR (mTAG72-CAR) construct. The extracellular spacer domain included the murine IgG1 region followed by a murine CD28 transmembrane domain66,67. The intracellular costimulatory signaling domain contained the murine 4-1 BB followed by a murine CD3ζ cytolytic domain as previously described⁶⁸. The CAR sequence was separated from a truncated murine CD19 gene (mCD19t) by a T2A ribosomal skip sequence. Murine membrane-bound IL12 was generated using murine p40 and p35 subunits sequences linked to a cell membrane anchoring murine CD28 transmembrane domain sequence. All retrovirus constructs were cloned into the pMYs retrovirus backbone under the control of a hybrid MMLV/MSCV promoter (Cell Biolabs Inc). Production of retrovirus used to transduce primary murine T cells were performed as previously described⁶⁹. Retrovirus was produced by transfecting the ecotropic retroviral packaging cell line, PLAT-E, with addition of mTAG72-CAR retrovirus backbone plasmid DNA using FuGENE HD transfection reagent (Promega). Viral supernatants were collected after 24, 36, and 48 h, pooled, and stored at −80° C. in aliquots for future T cell transductions. Control non-targeting murine mPSCA-CAR was generated as previously described.⁷⁰

### T Cell Isolation, Lentiviral Transduction, and Ex Vivo Expansion

Leukapheresis products were obtained from consented research participants (healthy donors) under protocols approved by the City of Hope Internal Review Board (IRB), and enriched for T cells as previously described^{38,58}. T cell activation and transduction was performed as described previously³⁸. Where indicated, we performed a second lentiviral transduction followed 24 hr after the first transduction. Cells were then ex vivo manufactured, enriched for CAR, and frozen as described previously³⁸. Purity and cell surface phenotype of CAR T cells were analyzed by flow cytometry using antibodies and methods as described below.

For mouse T cells, splenocytes were obtained by manual digestion of spleens from female C57BL/6j mice. Enrichment of T cells was performed by EasySep™ mouse T cell isolation kit per manufacturer's protocol (StemCell Technologies). Single or dual retroviral transductions with mTAG72-CAR, mPSCA-CAR and/or murine mbIL12 and subsequent expansion were performed as previously described, and cultured in cRPMI⁶⁹.

### Flow Cytometry

For flow cytometric analysis, cells were resuspended in FACS buffer (Hank's balanced salt solution without Ca²⁺, Mg²⁺, or phenol red (HBSS^{-/-}, Life Technologies) containing 2% FBS and 1×AA). Cells were incubated with primary antibodies for 30 min at 4° C. in the dark. For secondary staining, cells were washed twice prior to 30 min incubation at 4° C. in the dark with either Brilliant Violet 510 (BV510), fluorescein isothiocyanate (FITC), phycoerythrin (PE), peridinin chlorophyll protein complex (PerCP), PerCP-Cy5.5, PE-Cy7, allophycocyanin (APC), or APC-Cy7 (or APC-eFluor780)-conjugated antibodies. Antibodies against CD3 (BD Biosciences, Clone: SK7), CD4 (BD Biosciences, Clone: SK3), CD8 (BD Bosciences, Clone: SK1), CD19 (BD Biosciences, Clone: SJ25C1), mouse CD45 (BioLegend, Clone: 30-F11), CD45 (BD Biosciences, Clone: 2D1), CD69 (BD Biosciences, Clone: L78), CD137 (BD Biosciences, Clone: 4B4-1), biotinylated Protein L (GenScript USA) (25), TAG72 (Clone, muCC49), Donkey Anti-Rabbit Ig (Invitrogen), Goat Anti-Mouse Ig (BD Biosciences), and streptavidin (BD Biosciences) were used. Cell viability was determined using 4′, 6-diamidino-2-phenylindole (DAPI, Sigma). Flow cytometry was performed on a MACSQuant Analyzer 10 (Miltenyi Biotec), and the data was analyzed with FlowJo software (v10, TreeStar).

For intracellular flow cytometry, CAR T cells were thawed and rested in IL-2 (50 U/mL) & IL-15 (0.5 ng/mL) overnight at 1×10⁶ cells/mL. On the following day, CAR T cells were washed twice in 1× PBS and suspended at 1×10⁶ cells/mL in media without serum or cytokines. 1×10⁵ cells were plated per well in a 96-well plate to rest overnight. The next day, cells were stimulated with either soluble cytokine [IL-2 (50 U/mL), IL-15 (0.5 ng/mL), IL-12 (10 ng/mL)] or transferred to a high-binding 96-well plate pre-coated with indicated amounts of control or TAG72 antigen (BioRad). Reagents and buffers for flow cytometry processing were pre-chilled on ice unless otherwise stated. Following antigen stimulation, cells were washed with FACS buffer (supplemented with 0.1% sodium azide) and then fixed in pre-warmed 1× BD Phosflow Lyse/Fix buffer (558049) at 37° C. for 10 minutes. Cells were then washed with FACS buffer and if required, stained with the extracellular antibodies on ice for 30 minutes in the dark. Stained cells were washed and suspended in pre-chilled (−20° C.) BD Perm Buffer Ill (558050) and kept on ice for 30 minutes. Following a wash, cells were suspended in human FC block (Miltenyi Biotec Inc., FLP3330) and kept on ice for 30 minutes, washed and stained with intracellular antibodies: PE-pSTAT3, PE-pSTAT4, PE-pSTAT5 (Biolegend). Data was acquired on a MACSQuant Analyzer 16 cytometer (Miltenyi) and analyzed with FlowJo v10.8.

### In Vitro Tumor Killing and T Cell Functional Assays

For tumor cell killing assays, CAR T cells and tumor targets were co-cultured at indicated effector:tumor (E:T) ratios in complete X-VIVO (for human T cell assays) or cRPMI (for murine T cell assays) without cytokines in 96-well plates for the indicated time points and analyzed by flow cytometry as described above. Tumor cells were plated overnight prior to addition of T cells. Tumor cell killing by CAR T cells was calculated by comparing CD45-negative DAPI-negative (viable) cell counts relative targets co-cultured with untransduced (UTD) T cells. For tumor cell challenge assays, TAG72-CAR T cells engineered with or without mbIL12 were co-cultured with OV90 cells at 1:2 E:T ratio and rechallenged with OV90 cells every two days for up to five times. Similarly, HER2-CAR T cells engineered with or without mbIL12 were co-cultured with 468-HER2 tumor cells at 1:10 E:T ratio and rechallenged with 468-HER2 cells every three days. Remaining viable tumor cells and T cells were quantified by flow cytometry prior to every rechallenge and two or three days after the last rechallenge with tumor cells. For xCELLigence tumor cell killing assays, CAR T cells and tumor targets were co-cultured at indicated effector:tumor (E:T) ratios in complete X-VIVO without cytokines in 96-well plates for up to 10 days and analyzed by flow cytometry as described above.

To evaluate CAR T cell activity against normal tissue, normal tissue cells were co-cultured with CAR T cells at indicated E:T ratios. Patient-derived primary gastric cancer ascites (GAS1) and ovarian cancer ascites (OAS3 and OAS4) were thawed immediately and evaluated in T cell functional assays. After 48 hours, CD137 expression and cell killing was evaluated by flow cytometry, and supernatant was collected to quantify IFNγ by ELISA

For T cell activation assays, CAR T cells and tumor targets were co-cultured at the indicated E:T ratios in complete X-VIVO without cytokines in 96-well plates for the indicated time points and analyzed by flow cytometry for indicated markers of T cell activation. For T cell activation assays on plate-bound antigen, purified soluble TAG72 antigen (BioRad) was plated in duplicate at indicated TAG72 units overnight at 4° C. in 1× PBS in 96-well flat bottom high-affinity plates (Corning). Using a Bradford protein assay, the 20,000 units/mL stock solution of soluble TAG72 antigen was determined to be approximately 1.234 mg/mL of total protein. A designated number of TAG72-CAR T cells were then added in a fixed volume of 100 uL to each well and incubated for indicated times prior to collection of cells for analysis of activation markers (CD69, CD137) by flow cytometry. Supernatants were also collected for analysis of cytokine production. For T cell survival assays, T cells were plated at 1×10⁶cells/mL in X-VIVO 10% FBS with or without cytokines and counted every two days. Cell concentration was adjusted to 1×10⁶ cells/mL with fresh media following each count day.

### ELISA Cytokine Assays

Supernatants from tumor cell killing assays or CAR T cell activation assays on plate-bound TAG72 antigen were collected at indicated times and frozen at −20° C. for further use. Supernatants were then analyzed for secreted human IFNγ and IL-2 according to the Human IFNγ and IL-2 ELISA Ready-SET-GO!®; ELISA kit manufacturer's protocol, respectively. Plates were read at 450 nm using a Wallac Victor3 1420 Counter (Perkin-Elmer) and the Wallac 1420 Workstation software.

### Western Blotting Analysis

Cell pellets were thawed on ice. After thaw, cell pellets were resuspended in RIPA buffer consisting of 25 mM Tris-HCl (pH 8.5), 150 mM NaCl, 1 mM EDTA (pH 8.0), 1% (v/v) NP-40 substitute, 0.5% (w/v) Sodium Deoxycholate, 0.1% (w/v) SDS, 10 mM NaF, 1 mM NaOV, 10 mM p-glycerophosphate, and 1× of Halt Protease and Phosphotase Inhibitor Cocktail (Thermo Scientific). Lysates were incubated on ice for 30 min then centrifuged at 17,200 g for 20 minutes at 4° C. Lysate supernatant was transferred to a new tube and analyzed for total protein concentration by Braford protein assay. Laemmli sample buffer (BioRad) containing DTT (Sigma Aldrich) was added to proportional quantities of total protein and samples were boiled at 95° C. for 5 minutes. Protein was separated on a 7.5% Criterion TGX Precast Midi Protein Gel (BioRad) using the Criterion Cell (BioRad) and transferred to 0.2 um nitrocellulose blotting membrane (Genesee) in Tris-Glycine Transfer Buffer (Thermo Scientific) using the Trans-Blot Turbo Electrophoretic Transfer Cell (BioRad). Membranes were washed in deionized water, incubated in Ponceau S solution (Sigma Aldrich) to confirm protein transfer, and then washed in Tris-buffered saline containing 0.05% Tween20 (Sigma Aldrich) (TBST) for 1 minute. Membranes were then blocked for 1 hour at room temperature in blocking buffer containing 5% PhosphoBLOCKER blocking reagent (Cell Biolabs) in TBST. After blocking, membranes were transferred to blocking buffer containing primary antibodies and incubated overnight at 4° C. All primary antibodies were sourced from Cell Signaling Technology and included actin (1:2000), p44/42 MAPK (ERK1/2) (1:1000), pp44/42 MAPK (pERK1/2) (1:1000), SLP76 (1:1000), pSLP76 (1:1000), PLCy1 (1:1000), and pPLCy1 (1:1000). Membranes were washed in TBST and then incubated for 45 min at room temperature in blocking buffer containing either anti-rabbit or anti-mouse HRP-linked secondary antibody (Cell Signaling Technology). Membranes were washed in TBST and imaged on the ChemiDoc Imaging System using SuperSignal chemiluminescent substrate (Thermo 684 Scientific).

### In Vivo Studies

All animal experiments were performed under protocols approved by the City of Hope Institutional Animal Care and Use Committee. For in vivo intraperitoneal (i.p.) tumor studies, OVCAR3 and OV90 cells (5.0×10⁶) were prepared in a final volume of 500 uL HBSS^{-/-} and engrafted in >6 weeks old female NSG mice by i.p. injection. For subcutaneous (s.c.) tumor studies, OV90 cells (5×10⁶) were prepared in a final volume of 100 uL HBSS^{-/-} and injected under the skin of the abdomen of 6-8 weeks old female NSG mice. Tumor growth was monitored at least once a week via non-invasive bioluminescence imaging (Xenogen, LagoX) and flux signals were analyzed with Living Image software (Xenogen). For imaging, mice were i.p. injected with 150 uL D-luciferin potassium salt (Perkin Elmer) suspended in PBS at 4.29 mg/mouse. At day 8 for OV90 and day 14 for OVCAR3, mice were i.p. treated with indicated T cells (5×10⁶) in 500 uL final volume. Humane endpoints were used in determining survival. Mice were euthanized upon signs of distress such as a distended belly due to peritoneal ascites, labored or difficulty breathing, apparent weight loss, impaired mobility, or evidence of being moribund. At pre-determined time points or at moribund status, mice were euthanized and tissues and/or peritoneal ascites were harvested and processed for flow cytometry and immunohistochemistry as described herein.

Peripheral blood was collected from isoflurane-anesthetized mice by retro-orbital (RO) bleed through heparinized capillary tubes (Chase Scientific) into polystyrene tubes containing a heparin/PBS solution (1000 units/mL, Sagent Pharmaceuticals). Total volume of each RO blood draw (approximately 120 uL/mouse) was recorded. Red blood cells (RBCs) were lysed with 1× Red Cell Lysis Buffer (Sigma) according to manufacturer's protocol and then washed, stained, and analyzed by flow cytometry as described above. Cells from peritoneal ascites were collected from euthanized mice by injecting 5 mL cold 1× PBS into the i.p. cavity, which was drawn up via syringe and stored on ice until further processing. RBC-depleted ascites cells were washed, stained, and analyzed by flow cytometry using antibodies and methods as described above.

Serum from immune competent mouse studies was collected from non-heparinized blood collected by RO bleed as described above. Blood was kept at room temperature for 30 minutes followed by centrifugation at 6000×g for 10 minutes at 4° C., then aliquoted to multiple tubes and frozen at −80° C. until used for serum cytokine ELISA or chemistry analyses. Serum chemistry analysis was performed by running samples on a VETSCAN® VS2 Chemistry Analyzer (Zoetis), using the phenobarbital chemistry panel rotor (Zoetis) for ALT and AST quantification as described by manufacturer's protocol. Complete blood counts (CBC) analysis on whole blood collected from RO bleed, as described previously, was performed using a VETSCAN® HM5 Hematology Analyzer per manufacturer's protocol.

### Immunohistochemistry

Tumor tissue was fixed for up to 3 days in 4% paraformaldehyde (4% PFA, Boston BioProducts) and stored in 70% ethanol until further processing. Immunohistochemistry was performed by the Pathology Core at City of Hope. Briefly, paraffin-embedded sections (10 um) were stained with hematoxylin & eosin (H&E, Sigma-Aldrich), mouse anti-human CD3 (DAKO), mouse anti-human CD4 (DAKO), mouse anti-human CD8 (DAKO), and mouse anti-human TAG72 (AB16838, Abcam). Images were obtained using the Nanozoomer 2.OHT digital slide scanner and the associated NDP.view2 software (Hamamatzu).

### Statistical Analysis

Data are presented as means±standard error mean (SEM), unless otherwise stated. Statistical comparisons between groups were performed using the unpaired two-tailed Student's t test to calculate p value, unless otherwise stated.

### Additional Sequences

### Examples of Nucleotide Sequences Useful in a Human mbIL-12 Construct

Promotor Ef1a
(SEQ ID NO: 77)
GGATCTGCGATCGCTCCGGTGCCCGTCAGTGGGCAGAGCGCACATCGCCC
ACAGTCCCCGAGAAGTTGGGGGGAGGGGTCGGCAATTGAACCGGTGCCTA
GAGAAGGTGGCGCGGGGTAAACTGGGAAAGTGATGTCGTGTACTGGCTCC
GCCTTTTTCCCGAGGGTGGGGGAGAACCGTATATAAGTGCAGTAGTCGCC
GTGAACGTTCTTTTTCGCAACGGGTTTGCCGCCAGAACACAGCTGAAGCT
TCGAGGGGCTCGCATCTCTCCTTCACGCGCCCGCCGCCCTACCTGAGGCC
GCCATCCACGCCGGTTGAGTCGCGTTCTGCCGCCTCCCGCCTGTGGTGCC
TCCTGAACTGCGTCCGCCGTCTAGGTAAGTTTAAAGCTCAGGTCGAGACC
GGGCCTTTGTCCGGCGCTCCCTTGGAGCCTACCTAGACTCAGCCGGCTCT
CCACGCTTTGCCTGACCCTGCTTGCTCAACTCTACGTCTTTGTTTCGTTT
TCTGTTCTGCGCCGTTACAGATCCAAGCTGTGACCGGCGCCTAC
GMCSFRa\ss
(SEQ ID NO: 78)
GGCTAGCGCCACCATGCTGCTGCTCGTGACCAGCCTGCTGCTGTGCGAGC
TGCCCCACCCCGCCTTTCTGCTGATCCCC
hIL12\p40
(SEQ ID NO: 79)
ATATGGGAACTGAAGAAAGATGTTTATGTCGTAGAATTGGATTGGTATCC
GGATGCCCCTGGAGAAATGGTGGTCCTCACCTGTGACACCCCTGAAGAAG
ATGGTATCACCTGGACCTTGGACCAGAGCAGTGAGGTCTTAGGCTCTGGC
AAAACCCTGACCATCCAAGTCAAAGAGTTTGGAGATGCTGGCCAGTACAC
CTGTCACAAAGGAGGCGAGGTTCTAAGCCATTCGCTCCTGCTGCTTCACA
AAAAGGAAGATGGAATTTGGTCCACTGATATTTTAAAGGACCAGAAAGAA
CCCAAAAATAAGACCTTTCTAAGATGCGAGGCCAAGAATTATTCTGGACG
TTTCACCTGCTGGTGGCTGACGACAATCAGTACTGATTTGACATTCAGTG
TCAAAAGCAGCAGAGGCTCTTCTGACCCCCAAGGGGTGACGTGCGGAGCT
GCTACACTCTCTGCAGAGAGAGTCAGAGGGGACAACAAGGAGTATGAGTA
CTCAGTGGAGTGCCAGGAGGACAGTGCCTGCCCAGCTGCTGAGGAGAGTC
TGCCCATTGAGGTCATGGTGGATGCCGTTCACAAGCTCAAGTATGAAAAC
TACACCAGCAGCTTCTTCATCAGGGACATCATCAAACCTGACCCACCCAA
GAACTTGCAGCTGAAGCCATTAAAGAATTCTCGGCAGGTGGAGGTCAGCT
GGGAGTACCCTGACACCTGGAGTACTCCACATTCCTACTTCTCCCTGACA
TTCTGCGTTCAGGTCCAGGGCAAGAGCAAGAGAGAAAAGAAAGATAGAGT
CTTCACGGACAAGACCTCAGCCACGGTCATCTGCCGCAAAAATGCCAGCA
TTAGCGTGCGGGCCCAGGACCGCTACTATAGCTCATCTTGGAGCGAATGG
GCATCTGTGCCCTGCAGT
linker
(SEQ ID NO: 80)
GTTCCTGGAGTAGGGGTACCTGGGGTGGGC
hIL12\p35
(SEQ ID NO: 81)
GCCAGAAACCTCCCCGTGGCCACTCCAGACCCAGGAATGTTCCCATGCCT
TCACCACTCCCAAAACCTGCTGAGGGCCGTCAGCAACATGCTCCAGAAGG
CCAGACAAACTCTAGAATTTTACCCTTGCACTTCTGAAGAGATTGATCAT
GAAGATATCACAAAAGATAAAACCAGCACAGTGGAGGCCTGTTTACCATT
GGAATTAACCAAGAATGAGAGTTGCCTAAATTCCAGAGAGACCTCTTTCA
TAACTAATGGGAGTTGCCTGGCCTCCAGAAAGACCTCTTTTATGATGGCC
CTGTGCCTTAGTAGTATTTATGAAGACTCGAAGATGTACCAGGTGGAGTT
CAAGACCATGAATGCAAAGCTTCTGATGGATCCTAAGAGGCAGATCTTTC
TAGATCAAAACATGCTGGCAGTTATTGATGAGCTGATGCAGGCCCTGAAT
TTCAACAGTGAGACTGTGCCACAAAAATCCTCCCTTGAAGAACCGGATTT
TTATAAAACTAAAATCAAGCTCTGCATACTTCTTCATGCTTTCAGAATTC
GGGCAGTGACTATTGATAGAGTGATGAGCTATCTGAATGCTTCC
g3 linker
GGCGGAGGG
hCD28tm
(SEQ ID NO: 82)
TTCTGGGTGCTGGTGGTGGTGGGGGGGGTGCTGGCCTGCTACAGCCTGCT
GGTGACAGTGGCCTTCATCATCTTTTGGGTG
hCD28 cytoplasmic end
(SEQ ID NO: 83)
CGGAGCAAGCGG

### Examples of Nucleotide Sequences Useful in a Murine mbIL-12 Construct

Retrovirus backbone Promotor LTR/gag/pol/psi+
(specific to retrovirus packaging plasmid):
(SEQ ID NO: 84)
CTAGAGAACCATCAGATGTTTCCAGGGTGCCCCAAGGACCTGAAATGACC
CTGTGCCTTATTTGAACTAACCAATCAGTTCGCTTCTCGCTTCTGTTCGC
GCGCTTCTGCTCCCCGAGCTCAATAAAAGAGCCCACAACCCCTCACTCGG
GGCGCCAGTCCTCCGATTGACTGAGTCGCCCGGGTACCCGTATTCCCAAT
AAAGCCTCTTGCTGTTTGCATCCGAATCGTGGACTCGCTGATCCTTGGGA
GGGTCTCCTCAGATTGATTGACTGCCCACCTCGGGGGTCTTTCATTTGGA
GGTTCCACCGAGATTTGGAGACCCCAGCCCAGGGACCACCGACCCCCCCG
CCGGGAGGTAAGCTGGCCAGCGGTCGTTTCGTGTCTGTCTCTGTCTTTGT
GCGTGTTTGTGCCGGCATCTAATGTTTGCGCCTGCGTCTGTACTAGTTAG
CTAACTAGCTCTGTATCTGGCGGACCCGTGGTGGAACTGACGAGTTCGGA
ACACCCGGCCGCAACCCTGGGAGACGTCCCAGGGACTTCGGGGGCCGTTT
TTGTGGCCCGACCTGAGTCCAAAAATCCCGATCGTTTTGGACTCTTTGGT
GCACCCCCCTAATAGGAGGGATATGTGGTTCTGGTAGGAGACGAGAACCT
AAAACAGTTCCCGCCTCCGTCTGAATTTTTGCTTTCGGTTTGGGACCGAA
GCCGCGCCGCGCGTCTTGTCTGCTGCAGCATCGTTCTGTGTTGTCTCTGT
CTGACTGTGTTTCTGTATTTGTCTGAAAATTAGGGCCAGACTGTTACCAC
TCCCTTAAGTTTGACCTTAGGTCACTGGAAAGATGTCGAGCGGATCGCTC
ACAACCAGTCGGTAGATGTCAAGAAGAGACGTTGGGTTACCTTCTGCTCT
GCAGAATGGCCAACCTTTAACGTCGGATGGCCGCGAGACGGCACCTTTAA
CCGAGACCTCATCACCCAGGTTAAGATCAAGGTCTTTTCACCTGGCCCGC
ATGGACACCCAGACCAGGTCCCCTACATCGTGACCTGGGAAGCCTTGGCT
TTTGACCCCCCTCCCTGGGTCAAGCCCTTTGTACACCCTAAGCCTCCGCC
TCCTCTTCCTCCATCCGCCCCGTCTCTCCCCCTTGAACCTCCTCGTTCGA
CCCCGCCTCGATCCTCCCTTTATCCAGCCCTCACTCCTTCTCTAGGCGCC
CCCATATGGCCATATGAGATCTTATATGGGGCACCCCCGCCCCTTGTAAA
CTTCCCTGACCCTGACATGACAAGAGTTACTAACAGCCCCTCTCTCCAAG
CTCACTTACAGGCTCTCTACTTAGTCCAGCACGAAGTCTGGAGACCTCTG
GCGGCAGCCTACCAAGAACAACTGGACCGACCGGTGGTACCTCACCCTTA
CCGAGTCGGCGACACAGTGTGGGTCCGCCGACACCAGACTAAGAACCTAG
AACCTCGCTGGAAAGGACCTTACACAGTCCTGCTGACCACCCCCACCGCC
CTCAAAGTAGACGGCATCGCAGCTTGGATACACGCCGCCCACGTGAAGGC
TGCCGACCCCGGGGGTGGACCATCCTCTAGACTGCCG
Murine IL12 (p40 subunit)
(SEQ ID NO: 85)
ATGTGTCCTCAGAAGCTAACCATCTCCTGGTTTGCCATCGTTTTGCTGGT
GTCTCCACTCATGGCCATGTGGGAGCTGGAGAAAGACGTTTATGTTGTAG
AGGTGGACTGGACTCCCGATGCCCCTGGAGAAACAGTGAACCTCACCTGT
GACACGCCTGAAGAAGATGACATCACCTGGACCTCAGACCAGAGACATGG
AGTCATAGGCTCTGGAAAGACCCTGACCATCACTGTCAAAGAGTTTCTAG
ATGCTGGCCAGTACACCTGCCACAAAGGAGGCGAGACTCTGAGCCACTCA
CATCTGCTGCTCCACAAGAAGGAAAATGGAATTTGGTCCACTGAAATTTT
AAAAAATTTCAAAAACAAGACTTTCCTGAAGTGTGAAGCACCAAATTACT
CCGGACGGTTCACGTGCTCATGGCTGGTGCAAAGAAACATGGACTTGAAG
TTCAACATCAAGAGCAGTAGCAGTTCCCCTGACTCTCGGGCAGTGACATG
TGGAATGGCGTCTCTGTCTGCAGAGAAGGTCACACTGGACCAAAGGGACT
ATGAGAAGTATTCAGTGTCCTGCCAGGAGGATGTCACCTGCCCAACTGCC
GAGGAGACCCTGCCCATTGAACTGGCGTTGGAAGCACGGCAGCAGAATAA
ATATGAGAACTACAGCACCAGCTTCTTCATCAGGGACATCATCAAACCAG
ACCCGCCCAAGAACTTGCAGATGAAGCCTTTGAAGAACTCACAGGTGGAG
GTCAGCTGGGAGTACCCTGACTCCTGGAGCACTCCCCATTCCTACTTCTC
CCTCAAGTTCTTTGTTCGAATCCAGCGCAAGAAAGAAAAGATGAAGGAGA
CAGAGGAGGGGTGTAACCAGAAAGGTGCGTTCCTCGTAGAGAAGACATCT
ACCGAAGTCCAATGCAAAGGCGGGAATGTCTGCGTGCAAGCTCAGGATCG
CTATTACAATTCCTCATGCAGCAAGTGGGCATGTGTTCCCTGCAGGGTCC
GATCC
Linker
(SEQ ID NO: 86)
GGCGGCGGCGGGAGTGGCGGCGGGGGTTCTGGCGGAGGCGGTAGC
Murine IL12 (p35 subunit)
(SEQ ID NO: 87)
AGGGTCATTCCAGTCTCTGGACCTGCCAGGTGTCTTAGCCAGTCCCGAAA
CCTGCTGAAGACCACAGATGACATGGTGAAGACGGCCAGAGAAAAACTGA
AACATTATTCCTGCACTGCTGAAGACATCGATCATGAAGACATCACACGG
GACCAAACCAGCACATTGAAGACCTGTTTACCACTGGAACTACACAAGAA
CGAGAGTTGCCTGGCTACTAGAGAGACTTCTTCCACAACAAGAGGGAGCT
GCCTGCCCCCACAGAAGACGTCTTTGATGATGACCCTGTGCCTTGGTAGC
ATCTATGAGGACTTGAAGATGTACCAGACAGAGTTCCAGGCCATCAACGC
AGCACTTCAGAATCACAACCATCAGCAGATCATTCTAGACAAGGGCATGC
TGGTGGCCATCGATGAGCTGATGCAGTCTCTGAATCATAATGGCGAGACT
CTGCGCCAGAAACCTCCTGTGGGAGAAGCAGACCCTTACAGAGTGAAAAT
GAAGCTCTGCATCCTGCTTCACGCCTTCAGCACCCGCGTCGTGACCATCA
ACAGGGTGATGGGCTATCTGAGCTCCGCC
g3 linker
GGCGGAGGG
mCD28tm
(SEQ ID NO: 88)
TTTTGGGCACTGGTCGTGGTTGCTGGAGTCCTGTTTTGTTATGGCTTGCT
AGTGACAGTGGCTCTTTGTGTTATCTGGACA
mCD28 cytoplasmic end
(SEQ ID NO: 89)
AATAGTAGAAGG

1 Frigault, M. J. & Maus, M. V. State of the art in CAR T cell therapy for CD19+ B cell malignancies. J Clin Invest 130, 1586-1594, doi:10.1172/JC1129208 (2020).
2 June, C. H. & Sadelain, M. Chimeric Antigen Receptor Therapy. N Engl J Med 379, 64-73, doi:10.1056/NEJMra1706169 (2018).
3 Hong, M., Clubb, J. D. & Chen, Y. Y. Engineering CAR-T Cells for Next-Generation Cancer Therapy. Cancer Cell 38, 473-488, doi:10.1016/j.ccell.2020.07.005 (2020).
4 Priceman, S. J., Forman, S. J. & Brown, C. E. Smart CARs engineered for cancer immunotherapy. Curr Opin Oncol 27, 466-474, doi:10.1097/CCO.0000000000000232 (2015).
5 Schmidts, A. & Maus, M. V. Making CAR T Cells a Solid Option for Solid Tumors. Front Immunol 9, 2593, doi:10.3389/fimmu.2018.02593 (2018).
6 Sterner, R. C. & Sterner, R. M. CAR-T cell therapy: current limitations and potential strategies. Blood Cancer J 11, 69, doi:10.1038/s41408-021-00459-7 (2021).
7 van der Stegen, S. J., Hamieh, M. & Sadelain, M. The pharmacology of second-generation chimeric antigen receptors. Nat Rev Drug Discov 14, 499-509, doi:10.1038/nrd4597 (2015).
8 Larson, R. C. & Maus, M. V. Recent advances and discoveries in the mechanisms and functions of CAR T cells. Nat Rev Cancer 21, 145-161, doi:10.1038/s41568-020-00323-z (2021).
9 Liu, X. et al. Affinity-Tuned ErbB2 or EGFR Chimeric Antigen Receptor T Cells Exhibit an Increased Therapeutic Index against Tumors in Mice. Cancer Res 75, 3596-3607, doi:10.1158/0008-5472.CAN-15-0159 (2015).
10 Heitzeneder, S. et al. GPC2-CAR T cells tuned for low antigen density mediate potent activity against neuroblastoma without toxicity. Cancer Cell 40, 53-69 e59, doi:10.1016/j.ccell.2021.12.005 (2022).
11 Jonnalagadda, M. et al. Chimeric antigen receptors with mutated IgG4 Fc spacer avoid fc receptor binding and improve T cell persistence and antitumor efficacy. Mol Ther 23, 757-768, doi:10.1038/mt.2014.208 (2015).
12 Lee, S. Y. et al. Preclinical Optimization of a CD20-specific Chimeric Antigen Receptor Vector and Culture Conditions. J Immunother 41, 19-31, doi:10.1097/CJI.0000000000000199 (2018).
13 Kunkele, A. et al. Functional Tuning of CARs Reveals Signaling Threshold above Which CD8+ CTL Antitumor Potency Is Attenuated due to Cell Fas-FasL-Dependent AICD. Cancer Immunol Res 3, 368-379, doi:10.1158/2326-6066.CIR-14-0200 (2015).
14 Fujiwara, K. et al. Hinge and Transmembrane Domains of Chimeric Antigen Receptor Regulate Receptor Expression and Signaling Threshold. Cells 9, doi:10.3390/cells9051182 (2020).
15 Bridgeman, J. S. et al. The optimal antigen response of chimeric antigen receptors harboring the CD3zeta transmembrane domain is dependent upon incorporation of the receptor into the endogenous TCR/CD3 complex. J Immunol 184, 6938-6949, doi:10.4049/jimmunol.0901766 (2010).
16 Zhao, Z. et al. Structural Design of Engineered Costimulation Determines Tumor Rejection Kinetics and Persistence of CAR T Cells. Cancer Cell 28, 415-428, doi:10.1016/j.ccell.2015.09.004 (2015).
17 Guedan, S. et al. Single residue in CD28-costimulated CAR-T cells limits long-term persistence and antitumor durability. J Clin Invest 130, 3087-3097, doi:10.1172/JC1133215 (2020).
18 Salter, A. I. et al. Phosphoproteomic analysis of chimeric antigen receptor signaling reveals kinetic and quantitative differences that affect cell function. Sci Signal 11, doi:10.1126/scisignal.aat6753 (2018).
19 Yu, S., Yi, M., Qin, S. & Wu, K. Next generation chimeric antigen receptor T cells: safety strategies to overcome toxicity. Mol Cancer 18, 125, doi:10.1186/s12943-019-1057-4 (2019).
20 Lim, W. A. & June, C. H. The Principles of Engineering Immune Cells to Treat Cancer. Cell 168, 724-740, doi:10.1016/j.cell.2017.01.016 (2017).
21 Bell, M. & Gottschalk, S. Engineered Cytokine Signaling to Improve CAR T Cell Effector Function. Front Immunol 12, 684642, doi:10.3389/fimmu.2021.684642 (2021).
22 Brown, C. E. et al. Regression of Glioblastoma after Chimeric Antigen Receptor T-Cell Therapy. N Engl J Med 375, 2561-2569, doi:10.1056/NEJMoa1610497 (2016).
23 Priceman, S. J. et al. Regional Delivery of Chimeric Antigen Receptor-Engineered T Cells Effectively Targets HER2(+) Breast Cancer Metastasis to the Brain. Clin Cancer Res 24, 95-105, doi:10.1158/1078-0432.CCR-17-2041 (2018).
24 Wang, X. et al. The Cerebroventricular Environment Modifies CAR T Cells for Potent Activity against Both Central Nervous System and Systemic Lymphoma. Cancer Immunol Res 9, 75-88, doi:10.1158/2326-6066.CIR-20-0236 (2021).
25 Brown, C. E. et al. Optimization of IL13Ralpha2-Targeted Chimeric Antigen Receptor T Cells for Improved Anti-tumor Efficacy against Glioblastoma. Mol Ther 26, 31-44, doi:10.1016/j.ymthe.2017.10.002 (2018).
26 Vitanza, N. A. et al. Locoregional infusion of HER2-specific CAR T cells in children and young adults with recurrent or refractory CNS tumors: an interim analysis. Nat Med 27, 1544-1552, doi:10.1038/s41591-021-01404-8 (2021).
27 Adusumilli, P. S. et al. Regional delivery of mesothelin-targeted CAR T cell therapy generates potent and long-lasting CD4-dependent tumor immunity. Sci Transl Med 6, 261 ra151, doi:10.1126/scitranslmed.3010162 (2014).
28 Adusumilli, P. S. et al. A Phase I Trial of Regional Mesothelin-Targeted CAR T-cell Therapy in Patients with Malignant Pleural Disease, in Combination with the Anti-PD-1 Agent Pembrolizumab. Cancer Discov 11, 2748-2763, doi:10.1158/2159-8290.CD-21-0407 (2021).
29 Tchou, J. et al. Safety and Efficacy of Intratumoral Injections of Chimeric Antigen Receptor (CAR) T Cells in Metastatic Breast Cancer. Cancer Immunol Res 5, 1152-1161, doi:10.1158/2326-6066.CIR-17-0189 (2017).
30 Yeku, O. O., Purdon, T. J., Koneru, M., Spriggs, D. & Brentjens, R. J. Armored CAR T cells enhance antitumor efficacy and overcome the tumor microenvironment. Sci Rep 7, 10541, doi:10.1038/s41598-017-10940-8 (2017).
31 Murad, J. P. et al. Effective Targeting of TAG72(+) Peritoneal Ovarian Tumors via Regional Delivery of CAR-Engineered T Cells. Front Immunol 9, 2268, doi:10.3389/fimmu.2018.02268 (2018).
32 Luo, H. et al. Coexpression of IL7 and CCL21 Increases Efficacy of CAR-T Cells in Solid Tumors without Requiring Preconditioned Lymphodepletion. Clin Cancer Res 26, 5494-5505, doi:10.1158/1078-0432.CCR-20-0777 (2020).
33 Hege, K. M. et al. Safety, tumor trafficking and immunogenicity of chimeric antigen receptor (CAR)-T cells specific for TAG-72 in colorectal cancer. J Immunother Cancer 5, 22, doi:10.1186/s40425-017-0222-9 (2017).
34 Yoon, S. O. et al. Construction, affinity maturation, and biological characterization of an anti-tumor-associated glycoprotein-72 humanized antibody. J Biol Chem 281, 6985-6992, doi:10.1074/jbc.M511165200 (2006).
35 De Pascalis, R. et al. In vitro affinity maturation of a specificity-determining region-grafted humanized anticarcinoma antibody: isolation and characterization of minimally immunogenic high-affinity variants. Clin Cancer Res 9, 5521-5531 (2003).
36 Abate-Daga, D. & Davila, M. L. CAR models: next-generation CAR modifications for enhanced T-cell function. Mol Ther Oncolytics 3, 16014, doi:10.1038/mto.2016.14 (2016).
37 Long, A. H. et al. 4-1 BB costimulation ameliorates T cell exhaustion induced by tonic signaling of chimeric antigen receptors. Nat Med 21, 581-590, doi:10.1038/nm.3838 (2015).
38 Priceman, S. J. et al. Co-stimulatory signaling determines tumor antigen sensitivity and persistence of CAR T cells targeting PSCA+ metastatic prostate cancer. Oncoimmunology 7, e1380764, doi:10.1080/2162402X.2017.1380764 (2018).
39 Alizadeh, D. et al. IFNgamma Is Critical for CAR T Cell-Mediated Myeloid Activation and Induction of Endogenous Immunity. Cancer Discov 11, 2248-2265, doi:10.1158/2159-8290.CD-20-1661 (2021).
40 Jain, M. D. et al. Tumor interferon signaling and suppressive myeloid cells are associated with CAR T-cell failure in large B-cell lymphoma. Blood 137, 2621-2633, doi:10.1182/blood.2020007445 (2021).
41 Textor, A. et al. Efficacy of CAR T-cell therapy in large tumors relies upon stromal targeting by IFNgamma. Cancer Res 74, 6796-6805, doi:10.1158/0008-5472.CAN-14-0079 (2014).
42 Zhang, L. et al. Tumor-infiltrating lymphocytes genetically engineered with an inducible gene encoding interleukin-12 for the immunotherapy of metastatic melanoma. Clin Cancer Res 21, 2278-2288, doi:10.1158/1078-0432.CCR-14-2085 (2015).
43 Sridhar, P. & Petrocca, F. Regional Delivery of Chimeric Antigen Receptor (CAR) T-Cells for Cancer Therapy. Cancers (Basel) 9, doi:10.3390/cancers9070092 (2017).
44 Koneru, M., Purdon, T. J., Spriggs, D., Koneru, S. & Brentjens, R. J. IL-12 secreting tumor-targeted chimeric antigen receptor T cells eradicate ovarian tumors in vivo. Oncoimmunology 4, e994446, doi:10.4161/2162402X.2014.994446 (2015).
Henry, C. J., Ornelles, D. A., Mitchell, L. M., Brzoza-Lewis, K. L. & Hiltbold, E. M. IL-12 produced by dendritic cells augments CD8+ T cell activation through the production of the chemokines CCL1 and CCL17. J Immunol 181, 8576-8584, doi:10.4049/jimmunol.181.12.8576 (2008).
46 Guedan, S., Calderon, H., Posey, A. D., Jr. & Maus, M. V. Engineering and Design of Chimeric Antigen Receptors. Mol Ther Methods Clin Dev 12, 145-156, doi:10.1016/j.omtm.2018.12.009 (2019).
47 Miller, I. C. et al. Enhanced intratumoural activity of CAR T cells engineered to produce immunomodulators under photothermal control. Nat Biomed Eng 5, 1348-1359, doi:10.1038/s41551-021-00781-2 (2021).
48 Huang, Z. et al. Engineering light-controllable CAR T cells for cancer immunotherapy. Sci Adv6, eaay9209, doi:10.1126/sciadv.aay9209 (2020).
49 Prinzing, B. & Krenciute, G. Hypoxia-inducible CAR expression: An answer to the on-target/off-tumor dilemma? Cell Rep Med 2, 100244, doi:10.1016/j.xcrm.2021.100244 (2021).
50 Roybal, K. T. et al. Precision Tumor Recognition by T Cells With Combinatorial Antigen-Sensing Circuits. Cell 164, 770-779, doi:10.1016/j.cell.2016.01.011 (2016).
51 Srivastava, S. et al. Logic-Gated ROR1 Chimeric Antigen Receptor Expression Rescues T Cell-Mediated Toxicity to Normal Tissues and Enables Selective Tumor Targeting. Cancer Cell 35, 489-503 e488, doi:10.1016/j.ccell.2019.02.003 (2019).
52 Giordano-Attianese, G. et al. A computationally designed chimeric antigen receptor provides a small-molecule safety switch for T-cell therapy. Nat Biotechnol 38, 426-432, doi:10.1038/s41587-019-0403-9 (2020).
53 Zhang, L. et al. Improving adoptive T cell therapy by targeting and controlling IL-12 expression to the tumor environment. Mol Ther 19, 751-759, doi:10.1038/mt.2010.313 (2011).
54 Zhang, L. et al. Enhanced efficacy and limited systemic cytokine exposure with membrane-anchored interleukin-12 T-cell therapy in murine tumor models. J Immunother Cancer 8, doi:10.1136/jitc-2019-000210 (2020).
55 Hurton, L. V. et al. Tethered IL-15 augments antitumor activity and promotes a stem-cell memory subset in tumor-specific T cells. Proc Natl Acad Sci USA 113, E7788-E7797, doi:10.1073/pnas.1610544113 (2016).
56 Hu, J. et al. Cell membrane-anchored and tumor-targeted IL-12 (attIL12)-T cell therapy for eliminating large and heterogeneous solid tumors. J Immunother Cancer 10, doi:10.1136/jitc-2021-003633 (2022).
57 Agarwal, Y. et al. Intratumourally injected alum-tethered cytokines elicit potent and safer local and systemic anticancer immunity. Nat Biomed Eng 6, 129-143, doi:10.1038/s41551-021-00831-9 (2022).
58 Siddiqi, T. et al. CD19-directed CAR T-cell therapy for treatment of primary CNS lymphoma. Blood Adv 5, 4059-4063, doi:10.1182/bloodadvances.2020004106 (2021).
64 Neman, J. et al. Human breast cancer metastases to the brain display GABAergic properties in the neural niche. Proceedings of the National Academy of Sciences of the U.S.A. 111, 984-989, doi:10.1073/pnas.1322098111 (2014).
65 Ogawa, T. et al. ST6GALNAC1 plays important roles in enhancing cancer stem phenotypes of colorectal cancer via the Akt pathway. Oncotarget 8, 112550-112564, doi:10.18632/oncotarget.22545 (2017).
66 Kern, P., Hussey, R. E., Spoerl, R., Reinherz, E. L. & Chang, H.-C. Expression, Purification, and Functional Analysis of Murine Ectodomain Fragments of CD8αα and CD8αβ Dimers. Journal of Biological Chemistry 274, 27237-27243, doi:10.1074/jbc.274.38.27237 (1999).
67 Classon, B. J. et al. The hinge region of the CD8 alpha chain: structure, antigenicity, and utility in expression of immunoglobulin superfamily domains. Int Immunol 4, 215-225, doi:10.1093/intimm/4.2.215 (1992).
68 Kochenderfer, J. N., Yu, Z., Frasheri, D., Restifo, N. P. & Rosenberg, S. A. Adoptive transfer of syngeneic T cells transduced with a chimeric antigen receptor that recognizes murine CD19 can eradicate lymphoma and normal B cells. Blood 116, 3875-3886, doi:10.1182/blood-2010-01-265041 (2010).
69 Park, A. K. et al. Effective combination immunotherapy using oncolytic viruses to deliver CAR targets to solid tumors. Sci Transl Med 12, doi:10.1126/scitranslmed.aaz1863 (2020).
70 Murad, J. P. et al. Pre-conditioning modifies the TME to enhance solid tumor CAR T;cell efficacy and endogenous protective immunity. Molecular Therapy, doi:10.1016/j.ymthe.2021.02.024.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A nucleic acid molecule comprising a nucleotide sequence encoding a polypeptide comprising the amino acid sequence of SEQ ID NO: 6 or SEQ ID NO:7.

2. The nucleic acid molecule of claim 1, wherein the nucleotide sequence encodes a polypeptide comprising the amino acid sequence of SEQ ID NO: 6.

3. The nucleic acid molecule of claim 1, wherein the nucleotide sequence encodes a polypeptide comprising the amino acid sequence of SEQ ID NO: 7.

4. The nucleic acid molecule of claim 1, wherein the polypeptide further comprises a signal sequence.

5. The nucleic acid molecule of claim 4, wherein the signal sequence comprises the amino acid sequence of SEQ ID NO: 10 or SEQ ID NO: 11.

6. The nucleic acid molecule of claim 1 wherein the polypeptide comprises the amino acid sequence of SEQ ID NO: 8 or SEQ ID NO: 9.

7. The nucleic acid molecule of claim 1 wherein the polypeptide comprises the amino acid sequence of SEQ ID NO: 48 or SEQ ID NO: 49.

8. A population of human immune cells comprising the nucleic acid molecule of claim 1.

9. The population of human immune cells of claim 8, wherein the immune cells are T cells.
